(19) United States
(12) Patent Application Publication
(10) Pub. No.: US 2007/0124176 A1
(43) Pub. Date: May 31, 2007

(54) Computational and/or control systems and methods related to nutraceutical agent selection and dosing

(75) Inventors: Edward K. Y. Jung, Bellevue, WA (US); Royce A. Levien, Lexington, MA (US); Robert W. Lord, Seattle, WA (US); Mark A. Malamud, Seattle, WA (US); John D. Rinaldo JR., Bellevue, WA (US); Lowell L. Wood JR., Livermore, CA (US)

Correspondence Address: Searete LLC, Suite 110, 1756 - 114th Ave. S.E., Bellevue, WA, 98004, US

(73) Assignee: Searete LLC, a limited liability corporation of the State of Delaware

(21) Appl. No.: 11/523,809

(22) Filed: Sep. 18, 2006

Related U.S. Application Data
(63) Continuation-in-part of application No. 11/291,482, filed on Nov. 30, 2005.

(51) Int. Cl. G06F 19/00; A61B 5/00; G09B 23/28

(52) U.S. Cl. 705/3; 434/262; 600/300

(57) ABSTRACT

The present disclosure relates to computational and/or control systems and methods related to nutraceutical agent selection and dosing.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)).

### RELATED APPLICATIONS

For purposes of the USPTO extra-statutory requirements, the present application constitutes a continuation-in-part of U.S. Pat. application Ser. No. 11/453,571, entitled INDIVIDUALIZED PHARMACEUTICAL SELECTION AND PACKAGING, naming Edward K. Y. Jung, Royce A. Levien, Robert W. Lord, Mark A. Malamud, John D. Rinaldo, Jr., and Lowell L. Wood, Jr. as inventors, filed 14 Jun. 2006, which is currently co-pending, or is an application of which a currently co-pending application is entitled to the benefit of the filing date.

For purposes of the USPTO extra-statutory requirements, the present application constitutes a continuation-in-part of U.S. Pat. application Ser. No. 11/478,341, entitled COMPUTATIONAL AND/OR CONTROL SYSTEMS RELATED TO INDIVIDUALIZED NUTRACEUTICAL SELECTION AND PACKAGING, naming Edward K. Y. Jung, Royce A. Levien, Robert W. Lord, Mark A. Malamud, John D. Rinaldo, Jr., and Lowell L. Wood Jr. as inventors, filed 28 Jun. 2006, which is currently co-pending, or is an application of which a currently co-pending application is entitled to the benefit of the filing date.

For purposes of the USPTO extra-statutory requirements, the present application constitutes a continuation-in-part of U.S. Pat. application Ser. No. 11/478,296, entitled COMPUTATIONAL AND/OR CONTROL SYSTEMS RELATED TO INDIVIDUALIZED NUTRACEUTICAL SELECTION AND PACKAGING, naming Edward K. Y. Jung, Royce A. Levien, Robert W. Lord, Mark A. Malamud, Jolm D. Rinaldo, Jr., and Lowell L. Wood Jr. as inventors, filed 28 Jun. 2006, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 11/486,998, entitled COMPUTATIONAL AND/OR CONTROL SYSTEMS RELATED TO INDIVIDUALIZED PHARMACEUTICAL AND NUTRACEUTICAL SELECTION AND PACKAGING, naming Edward K. Y. Jung, Royce A. Levien, Robert W. Lord, Mark A. Malamud, John D. Rinaldo, Jr., and Lowell L. Wood Jr. as inventors, filed 14 Jul. 2006, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 11/486,973, entitled COMPUTATIONAL AND/OR CONTROL SYSTEMS RELATED TO INDIVIDUALIZED PHARMACEUTICAL AND NUTRACEUTICAL SELECTION AND PACKAGING, naming Edward K. Y. Jung, Royce A. Levien, Robert W. Lord, Mark A. Malamud, John D. Rinaldo, Jr., and Lowell L. Wood Jr. as inventors, filed 14 Jul. 2006, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 11/474,109, entitled CUSTOMIZED VISUAL MARKING FOR IVEDICATION LABELING, naming Edward K. Y. Jung, Royce A. Levien, Robert W. Lord, Mark A. Malamud, John D. Rinaldo, Jr., and Lowell L. Wood Jr. as inventors, filed 23 Jun. 2006, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 11/314,945, entitled GENERATING A REQUEST FROM A NUTRACEUTICAL INVENTORY, naming Edward K. Y. Jung, Royce A. Levien, Robert W. Lord, Mark A. Malamud, John D. Rinaldo, Jr., Clarence T. Tegreene, and Lowell L. Wood Jr. as inventors, filed 20 Dec. 2005, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 11/291,482, entitled GENERATING A NUTRACEUTICAL REQUEST FROM AN INVENTORY, naming Edward K. Y. Jung, Royce A. Levien, Robert W. Lord, Mark A. Malamud, John D. Rinaldo, Jr., Clarence T. Tegreene, and Lowell L. Wood Jr. as inventors, filed 30 Nov. 2005, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present applicant entity has provided above a specific reference to the application(s) from which priority is being claimed as recited by statute. Applicant entity understands that the statute is unambiguous in its specific reference language and does not require either a serial number or any characterization, such as “continuation” or “continuation-in-part,” for claiming priority to U.S. patent applications. Notwithstanding the foregoing, applicant entity understands that the USPTO's computer programs have certain data entry requirements, and hence applicant entity is designating the present application as a continuation-in-part of its parent applications as set forth above, but expressly points out that such designations are not to be construed in any way as any type of commentary and/or admission as to whether or not the present application contains any newmatter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### TECHNICAL FIELD

The present disclosure relates to computational and/or control systems and methods related to nutraceutical agent selection and dosing.

### SUMMARY

In some embodiments a method is provided that includes accepting data related to one or more specified goals of an individual; intaking information linked to one or more parameters associated with the one or more specified goals of the individual; selecting one or more nutraceutical agents in response to the intaking the information linked to the one or more parameters associated with the one or more specified goals of the individual; and indicating one or more dosages of the one or more nutraceutical agents in response to the selecting the one or more nutraceutical agents in response to the intaking the information linked to the one or more parameters associated with the one or more specified goals of the individual. In addition to the foregoing, other method aspects are described in the claims, drawings, and/or text forming a part of the present disclosure.

In some embodiments a method is provided that includes accepting data related to one or more specified goals of an individual; intaking information linked to one or more parameters associated with the one or more specified goals of the individual; and transmitting one or more signals in response to the intaking the information linked to the one or more parameters associated with the one or more specified goals of the individual. In addition to the foregoing, other method aspects are described in the claims, drawings, and/or text forming a part of the present disclosure.

In some embodiments a method is provided that includes receiving one or more signals transmitted in response to intaking information linked to one or more parameters associated with one or more specified goals of an individual; selecting one or more nutraceutical agents in response to the receiving the one or more signals transmitted in response to the intaking the information linked to the one or more parameters associated with the one or more specified goals of the individual; and indicating one or more dosages of the one or more nutraceutical agents in response to the selecting the one or more nutraceutical agents in response to the receiving the one or more signals transmitted in response to the intaking the information linked to the one or more parameters associated with the one or more specified goals of the individual. In addition to the foregoing, other method aspects are described in the claims, drawings, and/or text forming a part of the present disclosure.

In some embodiments a system is provided that includes circuitry for accepting data related to one or more specified goals of an individual; circuitry for intaking information linked to one or more parameters associated with the one or more specified goals of the individual; circuitry for selecting one or more nutraceutical agents responsive to the circuitry for intaking the information linked to the one or more parameters associated with the one or more specified goals of the individual; and circuitry for indicating one or more dosages of the one or more nutraceutical agents responsive to the circuitry for selecting the one or more nutraceutical agents responsive to the circuitry for intaking the information linked to the one or more parameters associated with the one or more specified goals of the individual. In addition to the foregoing, other system aspects are described in the claims, drawings, and/or text forming a part of the present disclosure.

In some embodiments a system is provided that includes circuitry for accepting data related to one or more specified goals of an individual; circuitry for intaking information linked to one or more parameters associated with the one or more specified goals of the individual; and circuitry for transmitting one or more signals responsive to the circuitry for intaking the information linked to the one or more parameters associated with the one or more specified goals of the individual. In addition to the foregoing, other system aspects are described in the claims, drawings, and/or text forming a part of the present disclosure.

In some embodiments a system is provided that includes circuitry for receiving one or more signals transmitted in response to intaking information linked to one or more parameters associated with one or more specified goals of an individual; circuitry for selecting one or more nutraceutical agents responsive to the circuitry for receiving the one or more signals transmitted in response to intaking the information linked to the one or more parameters associated with the one or more specified goals of the individual; and circuitry for indicating one or more dosages of the one or more nutraceutical agents responsive to the circuitry for selecting the one or more nutraceutical agents responsive to the circuitry for receiving the one or more signals transmitted in response to intaking the information linked to the one or more parameters associated with the one or more specified goals of the individual. In addition to the foregoing, other system aspects are described in the claims, drawings, and/or text forming a part of the present disclosure.

In some embodiments a system is provided that includes means for accepting data related to one or more specified goals of an individual; means for intaking information linked to one or more parameters associated with the one or more specified goals of the individual; means for selecting one or more nutraceutical agents responsive to the means for intaking the information linked to the one or more parameters associated with the one or more specified goals of the individual; and means for indicating one or more dosages of the one or more nutraceutical agents responsive to the means for selecting the one or more nutraceutical agents responsive to the means for intaking the information linked to the one or more parameters associated with the one or more specified goals of the individual. In addition to the foregoing, other system aspects are described in the claims, drawings, and/or text forming a part of the present disclosure.

In some embodiments a system is provided that includes means for accepting data related to one or more specified goals of an individual; means for intaking information linked to one or more parameters associated with the one or more specified goals of the individual; and means for transmitting one or more signals responsive to the means for intaking the information linked to the one or more parameters associated with the one or more specified goals of the individual. In addition to the foregoing, other system aspects are described in the claims, drawings, and/or text forming a part of the present disclosure.

In some embodiments a system is provided that includes means for receiving one or more signals transmitted in response to intaking information linked to one or more parameters associated with one or more specified goals of an individual; means for selecting one or more nutraceutical agents responsive to the means for receiving the one or more signals transmitted in response to intaking the information linked to the one or more parameters associated with the one or more specified goals of the individual; and means for indicating one or more dosages of the one or more nutraceutical agents responsive to the means for selecting the one or more nutraceutical agents responsive to the means for receiving the one or more signals transmitted in response to intaking the information linked to the one or more parameters associated with the one or more specified goals of the individual. In addition to the foregoing, other system aspects are described in the claims, drawings, and/or text forming a part of the present disclosure.

In some embodiments a system is provided that includes a signal-bearing medium bearing: one or more instructions for accepting data related to one or more specified goals of an individual; one or more instructions for intaking information linked to one or more parameters associated with the one or more specified goals of the individual; one or more instructions for selecting one or more nutraceutical agents in response to the intaking the information linked to the one or more parameters associated with the one or more specified goals of the individual; and one or more instructions for indicating one or more dosages of the one or more nutraceutical agents in response to the selecting the one or more nutraceutical agents in response to the intaking the information linked to the one or more parameters associated with the one or more specified goals of the individual. In addition to the foregoing, other system aspects are described in the claims, drawings, and/or text forming a part of the present disclosure.

In some embodiments a system is provided that includes a signal-bearing medium bearing: one or more instructions for accepting data related to one or more specified goals of an individual; one or more instructions for intaking information linked to one or more parameters associated with the one or more specified goals of the individual; and one or more instructions for transmitting one or more signals in response to the intaking the information linked to the one or more parameters associated with the one or more specified goals of the individual. In addition to the foregoing, other system aspects are described in the claims, drawings, and/or text forming a part of the present disclosure.

In some embodiments a system is provided that includes a signal-bearing medium bearing: one or more instructions for receiving one or more signals transmitted in response to intaking information linked to one or more parameters associated with one or more specified goals of an individual; one or more instructions for selecting one or more nutraceutical agents in response to the receiving the one or more signals transmitted in response to the intaking the information linked to the one or more parameters associated with the one or more specified goals of the individual; and one or more instructions for indicating one or more dosages of the one or more nutraceutical agents in response to the selecting the one or more nutraceutical agents in response to the receiving the one or more signals transmitted in response to the intaking the information linked to the one or more parameters associated with the one or more specified goals of the individual. In addition to the foregoing, other system aspects are described in the claims, drawings, and/or text forming a part of the present disclosure.

In some embodiments, means include but are not limited to circuitry and/or programming for effecting the herein-referenced functional aspects; the circuitry and/or programming can be virtually any combination of hardware, software, and/or firmware configured to effect the herein-referenced functional aspects depending upon the design choices of the system designer. In addition to the foregoing, other system aspects means are described in the claims, drawings, and/or text forming a part of the present disclosure.

In some embodiments, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer. In addition to the foregoing, other system aspects are described in the claims, drawings, and/or text forming a part of the present application.

The foregoing summary is illustrative only and is not intended to be in any way limiting. In addition to the illustrative aspects, embodiments, and features described above, further aspects, embodiments, and features will become apparent by reference to the drawings, claims, and the following detailed description.

### BRIEF DESCRIPTION OF THE FIGURES

FIG. 1 illustrates an example system 100 in which embodiments may be implemented.

FIG. 2 illustrates an operational flow 200 representing example operations related to methods for nutraceutical agent selection and dosing.

FIG. 3 illustrates alternative embodiments of the example operation flow of FIG. 2.

FIG. 4 illustrates alternative embodiments of the example operation flow of FIG. 2.

FIG. 5 illustrates alternative embodiments of the example operation flow of FIG. 2.

FIG. 6 illustrates alternative embodiments of the example operation flow of FIG. 2.

FIG. 7 illustrates alternative embodiments of the example operation flow of FIG. 2.

FIG. 8 illustrates alternative embodiments of the example operation flow of FIG. 2.

FIG. 9 illustrates an operational flow 900 representing example operations related to methods for nutraceutical agent selection and dosing.

FIG. 10 illustrates an operational flow 1000 representing example operations related to methods for nutraceutical agent selection and dosing.

FIG. 11 illustrates alternative embodiments of the example operation flow of FIG. 10.

FIG. 12 illustrates alternative embodiments of the example operation flow of FIG. 10.

FIG. 13 illustrates alternative embodiments of the example operation flow of FIG. 10.

FIG. 14 illustrates alternative embodiments of the example operation flow of FIG. 10.

FIG. 15 illustrates alternative embodiments of the example operation flow of FIG. 10.

FIG. 16 illustrates alternative embodiments of the example operation flow of FIG. 10.

FIG. 17 illustrates an operational flow 1700 representing example operations related to methods for nutraceutical agent selection and dosing.

FIG. 18 illustrates an operational flow 1800 representing example operations related to methods for nutraceutical agent selection and dosing.

FIG. 19 illustrates alternative embodiments of the example operation flow of FIG. 18.

FIG. 20 illustrates alternative embodiments of the example operation flow of FIG. 18.

FIG. 21 illustrates alternative embodiments of the example operation flow of FIG. 18.

FIG. 22 illustrates alternative embodiments of the example operation flow of FIG. 18.

FIG. 23 illustrates alternative embodiments of the example operation flow of FIG. 18.

FIG. 24 illustrates an operational flow 2400 representing example operations related to methods for nutraceutical agent selection and dosing.

FIG. 25 illustrates an example system 2500 in which embodiments may be implemented.

FIG. 26 illustrates an example system 2600 in which embodiments may be implemented.

FIG. 27 illustrates an example system 2700 in which embodiments may be implemented.

FIG. 28 illustrates an example system 2800 in which embodiments may be implemented.

FIG. 29 illustrates an example system 2900 in which embodiments may be implemented.

FIG. 30 illustrates an example system 3000 in which embodiments may be implemented.

FIG. 31 illustrates an example system 3100 in which embodiments may be implemented.

FIG. 32 illustrates an example system 3200 in which embodiments may be implemented.

FIG. 33 illustrates an example system 3300 in which embodiments may be implemented.

FIG. 34 illustrates an example system 3400 in which embodiments may be implemented.

FIG. 35 illustrates an example system 3500 in which embodiments may be implemented.

FIG. 36 illustrates an example system 3600 in which embodiments may be implemented.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

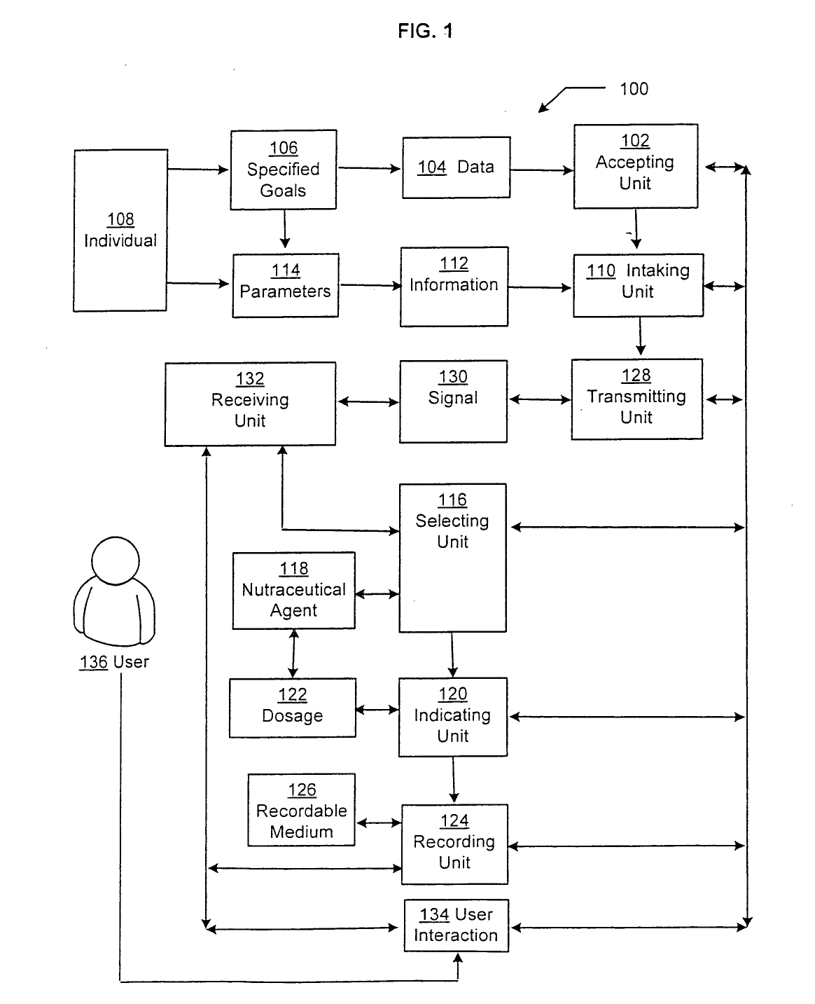

FIG. 1 illustrates an example system 100 in which embodiments may be implemented. In some embodiments, the system 100 is operable to provide a method and system for nutraceutical agent 118 selection and dosing. In some embodiments, the system 100 may be used by one or more individuals 108 to achieve one or more specified goals 106. In some embodiments, one or more accepting units 102 accept data 104 related to one or more specified goals 106 of one or more individuals 108. In some embodiments, one or more intaking units 110 intake information 112 linked to one or more parameters 114 associated with one or more specified goals 106 of an individual 108. In some embodiments, one or more selecting units 116 select one or more nutraceutical agents 118 in response to intaking information 112 linked to one or more parameters 114 associated with one or more specified goals 106 of an individual 108. In some embodiments, one or more indicating units 120 indicate one or more dosages 122 of one or more nutraceutical agents 118 in response to selecting the one or more nutraceutical agents 118 in response to intaking information 112 linked to one or more parameters 114 associated with the one or more specified goals 106 of the individual 108. In some embodiments, one or more recording units 124 record the results of: accepting data 104 related to one or more specified goals 106 of an individual 108, intaking information 112 linked to one or more parameters 114 associated with the one or more specified goals 106 of the individual 108, selecting one or more nutraceutical agents 118 in response to intaking information 112 linked to one or more parameters 114 associated with the one or more specified goals 106 of the individual 108, indicating one or more dosages 122 of the one or more nutraceutical agents 118 in response to selecting one or more nutraceutical agents 118 in response to intaking information 112 linked to one or more parameters 114 associated with the one or more specified goals 106 of the individual 108, or substantially any combination thereof, on a recordable-medium 126. In some embodiments, one or more transmitting units 128 transmit one or more signals 130 in response to intaking information 112 linked to one or more parameters 114 associated with one or more specified goals 106 of an individual 108. In some embodiments, one or more receiving units 132 receive one or more signals 130 transmitted in response to intaking information 112 linked to one or more parameters 114 associated with one or more specified goals 106 of the individual 108. In some embodiments, system 100 provides for user interaction 134 with one or more users 136. In some embodiments, such user interaction 134 can occur with one or more recording units 124, one or more indicating units 120, one or more selecting units 116, one or more receiving units 132, one or more transmitting units 128, one or more intaking units 110, one or more accepting units 102, and substantially any combination thereof. In some embodiments of system 100, one or more recording units 124, one or more indicating units 120, one or more selecting units 116, one or more receiving units 132, one or more transmitting units 128, one or more intaking units 110, and one or more accepting units 102, are hardwired together in substantially any combination. In some embodiments of system 100, one or more recording units 124, one or more indicating, units 120, one or more selecting units 116, one or more receiving units 132, one or more transmitting units 128, one or more intaking units 110, and one or more accepting units 102, may communicate through wireless connection. Numerous technologies may be used to establish and maintain wireless connections. In some embodiments of system 100, one or more recording units 124, one or more indicating units 120, one or more selecting units 116, one or more receiving units 132, one or more transmitting units 128, one or more intaking units 110, and one or more accepting units 102, may communicate through wireless connection and hardwired connections in substantially any combination. In some embodiments, user interaction 134 is provided through hardwired connections. In some embodiments, user interaction 134 is provided through wireless connections. In some embodiments, user interaction 134 is provided through hardwired and wireless connections.

### Accepting Unit 102

The system 100 can include one or more accepting units 102. In some embodiments, one or more accepting units 102 can be used to accept data 104 related to one or more specified goals 106 of one or more individuals 108. In some embodiments, one or more accepting units 102 can include a physical device with allows data 104 entry, such as a touchpad, keypad, hardwired telephone, and the like. In some embodiments, one or more accepting units 102 can include a wireless connection that allows the one or more accepting units 102 to accept data 104 from one or more individuals 108 through a wireless connection. For example, in some embodiments, one or more accepting units 102 may accept data 104 from one or more individuals 108 through use of a cellular telephone, a personal digital assistant, a wireless computer, and the like.

### Data 104/Specified Goals 106

The system 100 can include data 104 related to one or more specified goals 106 of an individual 108. In some embodiments, the data 104 can be related to one or more physical characteristics of an individual 108. In some embodiments, the data 104 can be related to one or more mental characteristics of an individual 108. In some embodiments, the data 104 can be related to one or more physiological characteristics of an individual 108. In some embodiments, the data 104 can be related to one or more performance characteristics of an individual 108. In some embodiments, the data 104 can be related to one or more psychological characteristics of an individual 108. Data 104 can be related to numerous types of specified goals 106. For example, in some embodiments, the data 104 can be related to attentiveness, alertness, test performance, relaxation, pain, fever, attractiveness, anxiety, fall, injury, accident, bite, bleeding, inflammation, infection, drowsiness, insomnia, discomfort, stress, grooming, appearance, capability, performance, improvement, enhancement, curtailment, wellbeing, vitality, vigor, disability, phobia, malady, psychosis, environmental extremes, environmental exposure, dysfunction, disease symptom, chronic condition, mental acuity, emotional behavior, physical prowess, addiction, obsession, therapy, remedy, behavior, nutrition, diet (includes weight-loss), exercise, immunization, prevention, diagnosis, subscription, regimen, social performance, social interaction, endurance, sexual attribute, sexual performance, age-related attribute, age-related condition, prophylaxis, treatment, and substantially any combination thereof.

### Individual 108

The system 100 can include one or more individuals 108. In some embodiments, an individual 108 may be afflicted with a diagnosed condition. For example, in some embodiments, an individual 108 may be afflicted with depression, anemia, obesity, insomnia, lower hormone levels, and the like. In some embodiments, an individual 108 may be afflicted with an undiagnosed condition. In some embodiments, such an undiagnosed condition may be an actual condition or a perceived condition.

### Intaking Unit 110

The system 100 can include one or more intaking units 110. In some embodiments, one or more intaking units 110 can be used to intake information 112 linked to one or more parameters 114 associated with one or more specified goals 106 of an individual 108. In some embodiments one or more intaking units 110 include instrumentation that can be used to assay one or more samples linked to an individual 108. In some embodiments, one or more intaking units 110 can be used to determine one or more levels of one or more metabolic indicators linked to an individual 108. In some embodiments, one or more intaking units 110 can be used to determine one or more levels of one or more metabolic activities linked to the individual 108. In some embodiments, one or more intaking units 110 can be used to determine one or more levels of one or more nutraceutical agents 118 linked to the individual 108. In some embodiments, one or more intaking units 110 can be used to determine one or more levels of one or more pharmaceutical agents linked to the individual 108. In some embodiments, one or more intaking units 110 can intake information 112 from another device. For example, in some embodiments, one or more intaking units 110 can intake information 112 provided by a diagnostic device. Such diagnostic devices include, but are not limited to, devices used to analyze bodily samples obtained from an individual 108 (i.e., blood, urine, saliva, synovial fluid, pleural fluid, peritoneal fluid, breath, skin, tissue, tears, mucus, genital products, hair, fecal material, and the like), devices used to analyze the appearance of an individual 108 (i.e., eye color, skin color, hair color, the presence or absence of bags under the eyes, presence or absence of hair, and the like), devices used to analyze a characteristic of the individual 108 (i.e., speech, reaction time, reflexes, temperature, eye dilation, retinal profile, height, weight, waistline, and the like), and other devices used to diagnose and/or analyze an individual 108. In some embodiments, one or more intaking units 110 can be included within system 100 through use of a hardwired connection. In some embodiments, one or more intaking units 110 can be included within system 100 through use of a wireless connection. In some embodiments, one or more intaking units 110 can be included within system 100 through use of a hardwired and a wireless connection. For example, in some embodiments, one or more intaking units 110 may intake.information 112 from a diagnostic or analytical device that is hardwired to the intaking unit 110. In other embodiments, one or more intaking units 110 may intake information 112 from a diagnostic or analytical device through use of a wireless connection.

### Selecting Unit 116

The system 100 can include one or more selecting units 116. In some embodiments, one or more selecting units 116 can be used to select one or more nutraceutical agents 118 in response to intaking information 112 linked to one or more parameters 114 associated with one or more specified goals 106 of an individual 108. In some embodiments, one or more selecting units 116 can be used to select one or more nutraceutical agents 118 to increase one or more levels of one or more components associated with the one or more specified goals 106 of the individual 108. In some embodiments, one or more selecting units 116 can be used to select one or more nutraceutical agents 118 to decrease one or more levels of one or more components associated with the one or more specified goals 106 of the individual 108. In some embodiments, one or more selecting units 116 can be used to select one or more nutraceutical agents 118 that stimulate one or more metabolic pathways linked to the individual 108. In some embodiments, one or more selecting units 116 can be used to select one or more nutraceutical agents 118 that inhibit one or more metabolic pathways linked to the individual 108. In some embodiments, one or more selecting units 116 can be used to select at least one vitamin, mineral, enzyme, amino acid, homeopathic supplement, toxin, homeopathic substance, traditional remedy, herbal supplement, glandular supplement, or substantially any combination thereof. In some embodiments, one or more selecting units 116 may access one or more databases that include information 112 with regard to nutraceutical agents 118, pharmaceutical agents, interaction of nutraceutical agents 118 with pharmaceutical agents, interaction of nutraceutical agents 118 and/or pharmaceutical agents with other substances, such as foods, beverages, over the counter drugs, and the like. In some embodiments, such databases may be included within one or more selecting units 116. In some embodiments, such databases may be remote from the one or more selecting units 116. Numerous criteria may be used to select nutraceutical agents 118. Examples of such criteria include, but are not limited to, price, availability, dosage form, interactions with other substances, method of administration (i.e., oral, nasal, pulmonary, and the like), time of administration, time of effect, duration of effect, single or multiple administration, and the like. In some embodiments, one or more selecting units 116 can be included within system 100 through use of a hardwired connection. In some embodiments, one or more selecting units 116 can be included within system 100 through use of a wireless connection. In some embodiments, one or more selecting units 116 can be included within system 100 through use of a hardwired and a wireless connection.

### Nutraceutical Agent 118

Nutraceutical agents 118 typically include natural, bioactive chemical compounds or any substance that is a plant, food, an extracted part of a food, that provides medical or health benefits but which generally fall outside regulations controlling pharmaceuticals. Included in this category of substances may be foods, isolated nutrients, supplements and herbs. Nutraceuticals are often referred to as phytochemicals or functional foods and include dietary supplements. Numerous nutraceuticals have been described (i.e., Roberts et al., Nutraceuticals: The Complete Encyclopedia of Supplements, Herbs, Vitamins, and Healing Foods, 1^{s} Edition, Perigee Trade (2001) and Susan G. Wynn, Emerging Therapies: Using Herbs and Nutraceuticals for Small Animals, American Animal Hospital Assn Press (1999); and Handbook of Nutraceuticals and Functional Foods., edited by Robert E. C. Wildman, CRC Press (2001)). Examples of nutraceutical agents 118 include, but are not limited to, Amino Acids, Terpenoids, Carotenoid Terpenoids (Lycopene, Beta-Carotene, Alpha-Carotene, Lutein, Zeaxanthin, Astaxanthin), Herbal Supplements, Homeopathic Supplements, Glandular Supplements, Non-Carotenoid Terpeniods (Perillyl Alcohol, Saponins, Terpeneol, Terpene Limonoids), Polyphenolics, Flavonoid Polyphenolics (Anthocyanins, Catechins, Isoflavones, Hesperetin, Naringin, Rutin, Quercetin, Silymarin, Tangeretin, Tannins), Phenolic Acids (Ellagic Acid, Chlorogenic Acid, Para-Coumaric Acid, Phytic Acid, Cinnamic Acid), Other Non-Flavonoid Polyphenolics (Curcumin, Resveratrol, Lignans), Glucosinolates, Isothiocyanates (Phenethyl Isothiocyanate, Benzyl Isothiocyanate, Sulforaphane), Indoles (Indole-3-Carbinol (I3C), Thiosulfonates, Phytosterols (Beta-Sitosterol), Anthraquinones (Senna, Barbaloin, Hypericin), Capsaicin, Piperine, Chlorophyll, Betaine, Pectin, Oxalic Acid, Acetyl-L-Camitine, Allantoin, Androsterondiol, Androsterondione, Betaine (Trimethylglycine), Caffeine, Calcium pyvurate (Pyruvic Acid), Carnitine, Carnosine, Carotene (alpha & beta), Carotenoid (Total for beadlets), Choline, Chlorogenic Acid, Cholic Acid (Ox Bile), Chondroitin Sulfate, Chondroitin Sulfate (Total Mucopolysaccharides), Cholestin, Chrysin, Coenzyme Q10 (Co-Q10), Conjugated Linoleic Acid (CLA), Corosolic Acid, Creatine, Dehydroepiandrosterone (DHEA), Dichlorophen, Diindolymethane (DIM), Dimethyglycine (DMG), Dimercapto Succinic Acid (DMSA), Ebselen, Ellagic Acid, Enzymes, Fisetin, Formonetin, Glucaric Acid (Glucarate), Glucosamine (HCl or Sulfate), Glucosamine (N-Acetyl), Glutathione (Reduced), Hesperidine, Hydroxy-3-Melethylbutyric Acid (HMIB), 5-Hydroxytryptophan (L-5-HTP), Indole-3-Carbinol, Inositol, Isothiocyanates, Linolenic Acid-Gamma (GLA), Lipoic Acid (alpha), Melatonin, Iviethylsulfonylmethane (MSM), Minerals, Naringin, Pancreatin, Para-aminobenzoic Acid (PABA), Paraben (methyl or propyl), Phenolics, Phosphatidylcholine (Lecithin), Phosphatidylserine, Phospholipids, Phytosterols, Pregersterone, Pregnenolone, Quercetin, Resveratrol, D-Ribose, Rutin, S-adenosylmethionine (SAM-e), Salicylic Acid, Sulforaphane, Tartaric Acid, Taxifolin, Tetrahydropalmatine, Thephyline, Theobromine, Tigogenin, Troxerutin, Tryptophan, Tocotrienol (alph, beta & gamma), Vitamins, Zeaxanthin, Gingo Biloba, Ginger, Cat's Claw, Hypericum, Aloe Vera, Evening Primrose, Garlic, Capsicum, Dong Quai, Ginseng, Feverview, Fenugreek, Echinacea, Green Tea, Marshmallow, Saw Palmetto, Tea Tree Oil, Payllium, Kava-Kava, Licorice Root, Manonia Aquifolium, Hawthorne, Hohimbr, Tumeric, Witch Hazel, Valerian, Mistletoe, Bilberry, Bee Pollen, Peppermint Oil, Beta-Carotene, Genistein, Lutein, Lycopene, the Polyphenols (bioflavonoids), and the like.

In some embodiments, nutraceutical agents 118 may include microbes (i.e., probiotics). Examples of such microbes include, but are not limited to, Lactobacillus acidophilus, Lactobacillus plantarum, Lactobacillus casei, Bifidobacterium bifidum, Bifidobacterium longum, Saccharomyces boulardii, Saccharomyces cerevisiae, and the like (i.e., Samuel and Gordon, A humanized gnotobiotic mouse model of host-archaeal-bacterial mutualism, PNAS, 103(26):10011-10016 (2006)). In some embodiments, nutraceutical agents 118 may include non-living microbes. For example, non-living Saccharomyces cerevisiae may be used as a source of vitamin B12. In some embodiments, recombinant microbes may be utilized as nutraceutical agents 11 8. For example, in some embodiments, microbes may be genetically modified to produce, or overexpress, one or more nutraceutical agents 118.

### Indicating Unit 120

The system 100 can include one or more indicating units 120. In some embodiments, one or more indicating units 120 can be used to indicate one or more dosages 122 of one or more nutraceutical agents 118 in response to selecting one or more nutraceutical agents 118 in response to intaking information 112 linked to one or more parameters 114 associated with one or more specified goals 106 of an individual 108. In some embodiments, one or more indicating units 120 indicate one or more dosages 122 of one or more nutraceutical agents 118 in human-readable format. In some embodiments, one or more indicating units 120 indicate one or more dosages 122 of one or more nutraceutical agents 118 in machine-readable format. In some embodiments, one or more indicating units 120 deposit one or more dosages 122 of one or more nutraceutical agents 118 on one or more labels. In some embodiments, one or more indicating units 120 dispense one or more nutraceutical agents 118. In some embodiments, one or more indicating units 120 dispense one or more nutraceutical agents 118 in unit dosage form. In some embodiments, one or more indicating units 120 dispense two or more nutraceutical agents 118 in a single administration form. In some embodiments, one or more indicating units 120 can be included within system 100 through use of a hardwired connection. In some embodiments, one or more indicating units 120 can be included within system 100 through use of a wireless connection. In some embodiments, one or more indicating units 120 can be included within system 100 through use of a hardwired and a wireless connection.

### Dosage 122

Dosages 122 may be expressed in numerous ways. In some embodiments, a dosage 122 may be expressed as an absolute quantity (i.e., 500 milligrams of a nutraceutical agent 118). In other embodiments, a dosage 122 may be expressed in accordance with the body weight of an individual 108 (i.e., 10 milligram nutraceutical agent 118 per kilogram body weight). In some embodiments, a dosage 122 may be expressed as a range of quantities (i.e., 10 milligrams to 100 milligrams of a nutraceutical agent 118). In some embodiments, a dosage 122 may be an amount of a nutraceutical agent 118 that produces a desired response when administered to a specific individual 108. For example, a dosage 122 of melatonin may be the amount of melatonin that induces sleep in a specific individual 108. The dosage 122 of a nutraceutical agent 118 may vary according to numerous considerations that include, but are not limited to, the route of administration, the age of the individual 108, the size of the individual 108, the metabolic characteristics of the individual 108, the condition of the individual 108, and the like. In some embodiments, the dosage 122 of a nutraceutical agent 118 may be determined that produces a measurable effect, such as a physical effect, a psychological effect, a physiological effect, and the like. Accordingly, in some embodiments, a dosage 122 may be expressed as an amount of a nutraceutical agent 118 that produces a mental response in an individual 108. For example, in some embodiments, a dosage 122 may be the amount of a nutraceutical agent 118 that produces a sensation of well-being when administered to an individual 108. In other embodiments, a dosage 122 may be the amount of a nutraceutical agent 118 that elevates the mood of an individual 108 to whom the nutraceutical is to be administered. Numerous additional criteria may be used to determine the dosage 122 of a nutraceutical for administration to an individual 108.

In some embodiments, one or more indicating units 120 can indicate one or more dosages 122 of one or more nutraceutical agents 118 and one or more formulations of the one or more nutraceutical agents 118. For example, in some embodiments, one or more indicating units 120 may indicate a formulation and dosage 122 of chromium. Presently, the most widely available chromium supplements are chromium salts such as chromium polynicotinate, chromium picolinate, and various chromium/amino acid chelates. Such formulations help increase the absorption and availability of chromium when compared to isolated chromium salts such as chromium chloride. The estimated safe and adequate daily dietary intake of chromium is 50-200 micrograms. Natural forms of supplemental chromium, such as chromium-rich yeast, may be absorbed somewhat more efficiently than inorganic forms of chromium, such as chromium chloride, found in some supplements. One ounce of brewer's yeast provides approximately 100-200 micrograms of chromium. Accordingly, in some embodiments, one or more indicating units 120 may indicate a dosage 122 of chromium and a corresponding formulation of the chromium. In another embodiment, one or more indicating units 120 may indicate a dosage 122 of vitamin A. For vitamin A deficiency syndromes, vitamin A may be orally supplemented at a dosage 122 of 600 micrograms for children aged 3 years or younger, 900 micrograms for children aged 4-8 years, 1700 micrograms for children aged 9-13 years, 800 micrograms for persons aged 14-18 years, and 3000 micrograms for all adults. Therapeutic doses for severe disease include 60,000 micrograms, which has been shown to reduce child mortality rates by 35-70%. One or more indicating units 120 may indicate dosages 122 for numerous types of nutraceutical agents 118 that are formulated in numerous ways.

### Transmitting Unit 128

The system 100 can include one or more transmitting units 128. In some embodiments, one or more transmitting units 128 can be used to transmit one or more signals 130 in response to intaking information 112 linked to one or more parameters 114 associated with one or more specified goals 106 of an individual 108. In some embodiments, one or more transmitting units 128 can be used to transmit one or more levels of one or more metabolic indicators linked to an individual 108. In some embodiments, one or more transmitting units 128 can be used to transmit one or more levels of one or more metabolic activities linked to an individual 108. In some embodiments, one or more transmitting units 128 can be used to transmit one or more levels of one or more nutraceutical agents 118 linked to an individual 108. In some embodiments, one or more transmitting units 128 can be used to transmit one or more levels of one or more pharmaceutical agents linked to an individual 108. In some embodiments, one or more transmitting units 128 can be used to transmit one or more parameters 114 associated with one or more specified goals 106 of an individual 108. In some embodiments, one or more transmitting units 128 can be used to transmit one or more instructions to select one or more nutraceutical agents 118 in response to intaking information 112 linked to one or more parameters 114 associated with one or more specified goals 106 of an individual 108. In some embodiments, one or more transmitting units 128 can be used to transmit one or more signals 130 to select one or more nutraceutical agents 118 to increase one or more levels of one or more components associated with one or more specified goals 106 of an individual 108. In some embodiments, one or more transmitting units 128 can be used to transmit one or more signals 130 to select one or more nutraceutical agents 118 to decrease one or more levels of one or more components associated with one or more specified goals 106 of an individual 108. In some embodiments, one or more transmitting units 128 can be used to transmit one or more signals 130 to select one or more nutraceutical agents 118 that stimulate one or more metabolic pathways linked to an individual 108. In some embodiments, one or more transmitting units 128 can be used to transmit one or more signals 130 to select one or more nutraceutical agents 118 that inhibit one or more metabolic pathways linked to an individual 108. In some embodiments, one or more transmitting units 128 can be used to transmit one or more signals 130 to select at least one vitamin, mineral, enzyme, amino acid, homeopathic supplement, toxin, homeopathic substance, traditional remedy, herbal supplement, glandular supplement, or substantially any combination thereof. In some embodiments, one or more transmitting units 128 can be included within system 100 through use of a hardwired connection. In some embodiments, one or more transmitting units 128 can be included within system 100 through use of a wireless connection. In some embodiments, one or more transmitting units 128 can be included within system 100 through use of a hardwired and a wireless connection.

### Signal 130

The system 100 may include one or more signals 130. Numerous types of signals 130 may be transmitted. Examples of such signals 130 include, but are not limited to, hardwired signals 130, wireless signals 130, infrared signals 130, optical signals 130, radiofrequency (RF) signals 130, audible signals 130, digital signals 130, analog signals 130, or substantially any combination thereof.

### Receiving Unit 132

The system 100 may include one or more receiving units 132. In some embodiments, one or more receiving units 132 receive one or more signals 130 transmitted in response to intaking information 112 linked to one or more parameters 114 associated with one or more specified goals 106 of an individual 108. In some embodiments, one or more receiving units 132 receive one or more signals 130 indicating one or more levels of one or more metabolic indicators linked to an individual 108. In some embodiments, one or more receiving units 132 receive one or more signals 130 indicating one or more metabolic activities linked to an individual 108. In some embodiments, one or more receiving units 132 receive one or more signals 130 indicating one or more levels of one or more nutraceutical agents 118 linked to an individual 108. In some embodiments, one or more receiving units 132 receive one or more signals 130 indicating one or more levels of one or more pharmaceutical agents linked to an individual 108. In some embodiments, one or more receiving units 132 receive one or more parameters 114 associated with one or more specified goals 106 of an individual 108. In some embodiments, one or more receiving units 132 receive one or more instructions to select one or more nutraceutical agents 118 in response to intaking information 112 linked to one or more parameters 114 associated with the one or more specified goals 106 of the individual 108. In some embodiments, one or more receiving units 132 receive one or more signals 130 to select one or more nutraceutical agents 118 to increase one or more levels of one or more components associated with one or more specified goals 106 of an individual 108. In some embodiments, one or more receiving units 132 receive one or more signals 130 to select one or more nutraceutical agents 118 to decrease one or more levels of one or more components associated with one or more specified goals 106 of an individual 108. In some embodiments, one or more receiving units 132 receive one or more signals 130 to select one or more nutraceutical agents 118 that stimulate one or more metabolic pathways linked to an individual 108. In some embodiments, one or more receiving units 132 receive one or more signals 130 to select one or more nutraceutical agents 118 that inhibit one or more metabolic pathways linked to an individual 108. In some embodiments, one or more receiving units 132 receive one or more signals 130 to select at least one vitamin, mineral, enzyme, amino acid, homeopathic supplement, toxin, homeopathic substance, traditional remedy, herbal supplement, glandular supplement, or substantially any combination thereof.

Receiving units 132 may receive numerous types of signals 130. Examples of such signals 130 include, but are not limited to, hardwired signals 130, wireless signals 130, infrared signals 130, optical signals 130, radiofrequency (RF) signals 130, auditory signals 130, digital signals 130, analog signals 130, or substantially any combination thereof.

### Recording Unit 124

The system 100 may include one or more recording units 124. In some embodiments, one or more recording units 124 can communicate with one or more accepting units 102, one or more intaking units 10, one or more transmitting units 128, one or more receiving units 132, one or more selecting units 116, one or more indicating units 120, one or more users 136, and/or substantially any combination thereof. Many types of recording units 114 may be used within system 100. Examples of Such recording devices include those that utilize a recordable medium 126 that includes, but is not limited to, many types of memory, optical disks, magnetic disks, magnetic tape, and the like.

### User Interaction 134/User 136

The system 100 may provide for user interaction 134. In some embodiments, a user 136 may interact with one or more accepting units 102, one or more intaking units 110, one or more transmitting units 128, one or more receiving units 132, one or more selecting units 116, one or more indicating units 120, one or more recording units 124, and/or substantially any combination thereof. The user 136 can interact through use of numerous technologies. For example, user interaction 134 can occur through use of hardwired methods, such as through use of a keyboard, use of wireless methods, use of the internet, and the like. In some embodiments, a user 136 is a health-care worker. Examples of such health-care workers include, but are not limited to, physicians, nurses, dieticians, pharmacists, and the like. In some embodiments, users 136 may include those persons who work in health-related fields, such as coaches, personal trainers, clerks at food supplement stores, clerks at grocery stores, and the like. In some embodiments, a user 136 is not human.

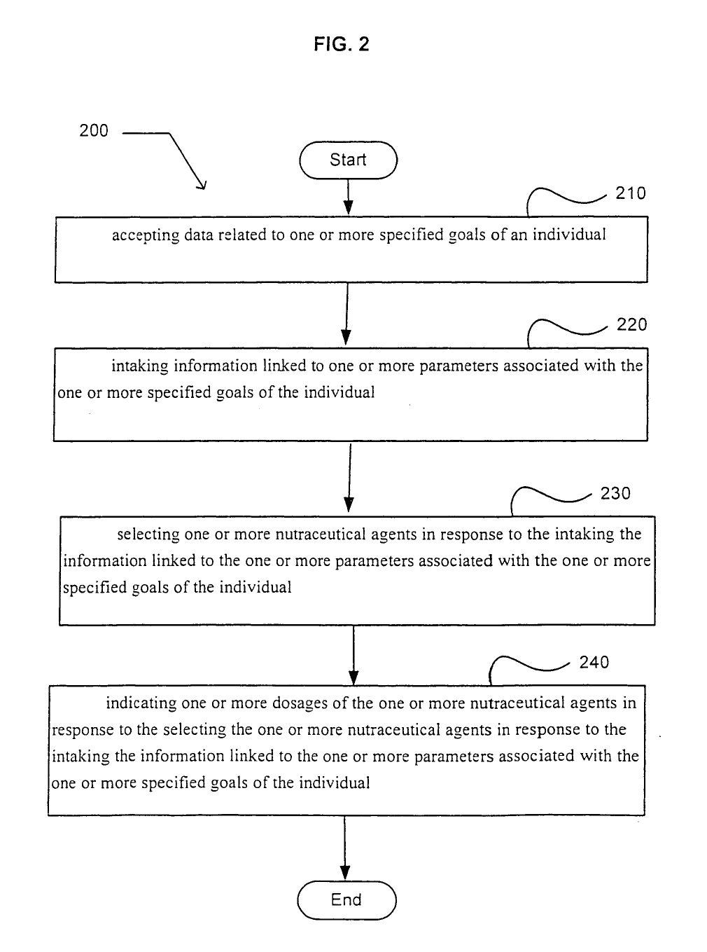

FIG. 2 illustrates an operational flow 200 representing examples of operations that are related to the performance of a method for nutraceutical agent 118 and dosing. In FIG. 2 and in following figures that include various examples of operations used during performance of the method, discussion and explanation may be provided with respect to the above-described example of FIG. 1, and/or with respect to other examples and contexts. However, it should be understood that the operations may be executed in a number of other environments and contexts, and/or modified versions of FIG. 1. Also, although the various operations are presented in the sequence(s) illustrated, it should be understood that the various operations may be performed in other orders than those which are illustrated, or may be performed concurrently.

After a start operation, the operational flow 200 includes an accepting operation 210 involving accepting data related to one or more specified goals of an individual. In some embodiments, one or more accepting units 102 may accept data 104 related to one or more specified goals 106 of an individual 108. The one or more specified goals 106 may be virtually any goal to be achieved, or attempted by, an individual 108 that may be affected by administration of one or more nutraceutical agents 118 to the individual 108. In some embodiments, administration of one or more nutraceutical agents 118 to an individual 108 may assist the individual 108 to achieve one or more specified goals 106. In some embodiments, administration of one or more nutraceutical agents 118 to an individual 108 may stimulate the individual 108 to achieve one or more specified goals 106. In some embodiments, administration of one or more nutraceutical agents 118 to an individual 108 may reduce an inhibition coupled to the individual 108 to promote achieving one or more specified goals 106 by the individual 108. Numerous examples of specified goals 106 of an individual 108 exist. In some embodiments, specified goals 106 of an individual 108 may be related to attentiveness, alertness, test performance, relaxation, pain, fever, attractiveness, anxiety, fall, injury, accident, bite, bleeding, inflammation, infection, drowsiness, insomnia, discomfort, stress, grooming, appearance, capability, performance, improvement, enhancement, curtailment, wellbeing, vitality, vigor, disability, phobia, malady, psychosis, environmental extremes, environmental exposure, dysfunction, disease symptom, chronic condition, mental acuity, emotional behavior, physical prowess, addiction, obsession, therapy, remedy, behavior, nutrition, diet, exercise, immunization, prevention, diagnosis, subscription, regimen, social performance, social interaction, endurance, sexual attribute, sexual performance, age-related attribute, age-related condition, prophylaxis, treatment, and substantially any combination thereof. In some embodiments, data 104 may include identification of one or more of the specified goals 106 of an individual 108. In some embodiments, data 104 may include characteristics of an individual 108. Examples of such data 104 may include, but are not limited to, physical characteristics, metabolic characteristics, financial characteristics, and the like. In some embodiments, data 104 may include, an individual's 108 height, weight, gender, kidney function, liver function, level of physical fitness, age, allergic response, metabolic level (i.e., resting metabolic rate and/or activity-related metabolic rate), disease state, body fat percentage, body mass index, personal health habits (i.e., smoking, alcohol consumption, diet, illegal drug use, and the like), family health history, insurance coverage, food supplement usage, nutraceutical agent 118 usage, non-prescription drug use, prescription drug use, pregnancy status, and the like. In some embodiments, the one or more characteristics may be specifically associated with an individual 108. As such, in some embodiments, the one or more characteristics may be unique to the individual 108 as opposed to being common to a group. For example, in some embodiments, an individual 108 may be a member of a group of persons who are diabetic while exhibiting one or more characteristics, such as metabolic characteristics, that are unique to the individual 108. Accordingly, in some embodiments, data 104 may be input that provides for selection of nutraceutical agents 118 in accordance with one or more characteristics and specified goals 106 of an individual 108.

The operational flow 200 includes an intaking operation 220 involving intaking information linked to one or more parameters associated with the one or more specified goals of the individual. In some embodiments, one or more intaking units 110 may intake information 112 linked to one or more parameters 114 associated with the one or more specified goals 106 of the individual 108. Numerous parameters 114 may be associated with one or more specified goals 106 of an individual 108. Examples of such parameters 114 include, but are not limited to, blood sugar levels, blood insulin levels, blood iron levels, hormone levels (amine-derived hormones: such as catecholamines (adrenaline, dopamine, noradrenaline); tryptophan derivatives (melatonin, serotonin); tyrosine derivatives (thyroxine and triuodothyronine); peptide hormones such as antimullerian hormone, adiponectin, adrenocorticotropic hormone, angiotensinogen and angiotensin, antidiuretic hormone, atrial-natriuretic peptide, calcitonin, cholecystokinin, corticotropin-releasing hormone, erythropoietin, follicle-stimulating hormone, gastrin, ghrelin, glucagons, gonadotropin-releasing hormone, growth hormone-releasing hormone, human chorionic gonadotropin, growth hormone, inhibin, insulin, insulin-like growth factor, luteinizing hormone, melanocyte stimulating hormone, neuropeptide Y. oxytocin, parathyroid hormone, prolactin, relaxin, secretin, somatostatin, thrombopoietin, thyroid-stimulating hormone, thyrotropin-releasing hormone; steroid hormones: Glucocorticoids (cortisol); Mineralocorticoids (aldosterone); sex steroids: androoens (testosterone, dehydroepiandrosterone, dehydroepiandrosterone sulfate, androstenedione, dihydrotestosterone); estrogens (estradiol); progestagens (progesterone and progestins); sterol hormones: vitamin D derivatives (calcitriol); lipid hormones (prostaglandins, leukotrienes, prostacyclin, and thromboxane)), nutraceutical agent 118 levels, pharmaceutical agent levels, cytokine levels, and the like (i.e., Fitzgerald et al., The Cytokine Facts Book, Academic Press, San Francisco, Second Edition, (2001)). Methods to gain information 112 with regard to components of biological systems are known (i.e., Clinical Laboratory Medicine, Lippincott Williams and Wilkins, Philadelphia, Second Edition, 2002). In some embodiments, one or more intaking units 110 may include instrumentation that provides for analysis of a sample obtained from an individual 108. For example, in some embodiments, an intaking unit 110 may be configured to intake a blood sample obtained from an individual 108 and analyze the blood sample to determine one or more parameters 114 associated with one or more specified goals 106 of an individual 108 (i.e., determine the level of free testosterone or the level of melatonin in a blood sample obtained from an individual 108). Numerous analytical technologies are known and may be included within one or more intaking units 110. Examples of such technologies include, but are not limited to, gas chromatography, mass spectrometry, atomic absorption, immunoassay based methods, microfluidic based methods, spectrophotometry (i.e., infrared, ultraviolet, fluorescence, and the like), surface plasmon resonance, fluorescence resonance energy transfer, and the like. In some embodiments, one or more intaking units 110 may intake information 112 linked to one or more parameters 114 associated with the one or more specified goals 106 of an individual 108 from a source that is independent of the one or more intaking units 110. In some embodiments, one or more intaking units 110 may intake information 112 linked to one or more parameters 114 associated with the one or more specified goals 106 of an individual 108 from a source that is coupled to the one or more intaking units 110.

The operational flow 200 includes a selecting operation 230 involving selecting one or more nutraceutical agents in response to the intaking the information linked to the one or more parameters associated with the one or more specified goals of the individual. In some embodiments, one or more selecting units 116 may select one or more nutraceutical agents 118 in response to the intaking information 112 linked to one or more parameters 114 associated with the one or more specified goals 106 of the individual 108. In some embodiments, one or more selecting units 116 may select one or more nutraceutical agents 118 in response to one or more intaking units 110. In some embodiments, one or more selecting units 116 may select one or more nutraceutical agents 118 in response to one or more accepting units 102. In some embodiments, one or more selecting units 116 may select one or more nutraceutical agents 118 in response to one or more intaking units 110 and one or more accepting units 102. Accordingly, in some embodiments, one or more selecting units 116 may select one or more nutraceutical agents 118 with regard to one or more characteristics of the individual 108 and one or more parameters 114 associated with one or more specified goals 106 of the individual 108. For example, in some embodiments, one or more selecting units 116 may select one or more nutraceutical agents 118 based on the age of the individual 108 and the level of testosterone in the individual's 108 blood. In other embodiments, one or more selecting units 116 may select one or more nutraceutical agents 118 based on insurance coverage held by an individual 108 and a specified goal 106 of the individual 108. Accordingly, numerous combinations of information 112 and data 104 may be used by one or more selecting units 116 to select one or more nutraceutical agents 118.

The operational flow 200 includes an indicating operation 240 involving indicating one or more dosages of the one or more nutraceutical agents in response to the selecting the one or more nutraceutical agents in response to the intaking the information linked to the one or more parameters associated with the one or more specified goals of the individual. In some embodiments, one or more indicating units 120 may indicate one or more dosages 122 of the one or more nutraceutical agents 118 in response to the intaking information 112 linked to one or more parameters 114 associated with the one or more specified goals 106 of the individual 108. In some embodiments, one or more indicating units 120 may indicate one or more dosages 122 of one or more nutraceutical agents 118 in human-readable format. In some embodiments, one or more indicating units 120 may indicate one or more dosages 122 of one or more nutraceutical agents 118 in machine-readable format. In some embodiments, one or more indicating units 120 may deposit one or more dosages 122 of one or more nutraceutical agents 118 on one or more labels. In some embodiments, one or more indicating units 120 may dispense one or more nutraceutical agents 118. In some embodiments, one or more indicating units 120 may dispense one or more nutraceutical agents 118 in unit dosage form. In some embodiments, one or more indicating units 120 may dispense two or more of one or more nutraccutical agents 118 in a single administration form.

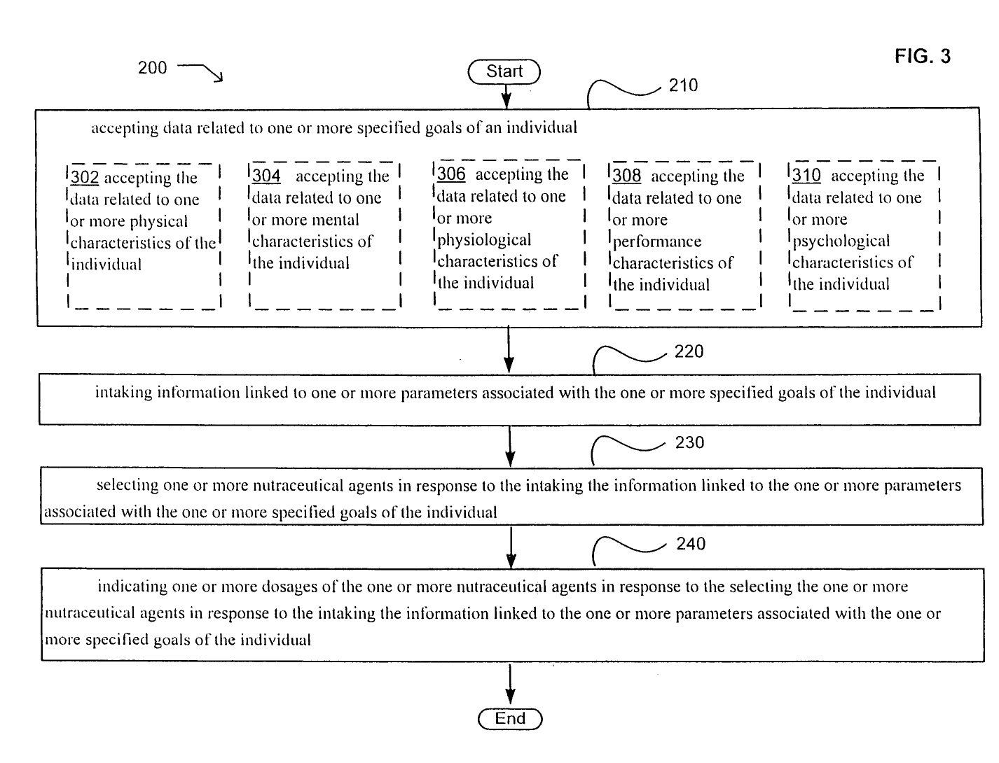

FIG. 3 illustrates alternative embodiments of the example operational flow 200 of FIG. 2. FIG. 3 illustrates example embodiments where the accepting operation 210 may include at least one additional operation. Additional operations may include an operation 302, an operation 304, an operation 306, an operation 308, and/or an operation 310.

At operation 302, the accepting operation 210 may include accepting the data related to one or more physical characteristics of the individual. In some embodiments, one or more accepting units 102 accept data 104 related to one or more physical characteristics of the individual 108.

One or more accepting units 102 may accept data 104 related to numerous physical characteristics of an individual 108. Examples of such physical characteristics include, but are not limited to, height, weight, gender, kidney function, liver function, level of physical fitness, age, allergic response, metabolic level (i.e., resting metabolic rate and/or activity-related metabolic rate), disease state, body fat percentage, body mass index, personal health habits (i.e., smoking, alcohol consumption, diet, illegal drug use, and the like), family health history, food supplement usage, nutraceutical agent 118 usage, non-prescription drug use, prescription drug use, pregnancy status, and the like. In some embodiments, such data 104 may be used to identify one or more nutraceutical aoents 118 that are compatible with an individual 108. For example, if an individual 108 is taking a selective serotonin reuptake inhibitor (SSRI), one or more nutraceutical agents 118 should not be selected that would interfere with the serotonin reuptake inhibitor.

At operation 304, the accepting operation 210 may include accepting the data related to one or more mental characteristics of the individual. In some embodiments, one or more accepting units 102 accept data 104 related to one or more mental characteristics of the individual 108.

One or more accepting units 102 may accept data 104 related to numerous mental characteristics of an individual 108. Examples of such mental characteristics include, but are not limited to, daily time periods when the mental processes of an individual 108 are most acute or least acute (i.e., morning, afternoon, evening, night), an individual's 108 sleep schedule, an individual's 108 daily schedule of activities (i.e., meetings, presentations, travel, athletic activity), and the like.

At operation 306, the accepting operation 210 may include accepting the data related to one or more physiological characteristics of the individual. In some embodiments, one or more accepting units 102 accept data 104 related to one or more physiological characteristics of the individual 108.

One or more accepting units 102 may accept data 104 related to numerous physiological characteristics of an individual 108. Examples of such physiological characteristics include, but are not limited to, the ability of an individual 108 to metabolize one or more nutraceutical agents 118, the ability of an individual 108 to metabolize one or more pharmaceutical agents, the response of an individual 108 to one or more pharmaceutical agents, the response of an individual 108 to one or more nutraceutical agents 118, the concentration or level of one or more metabolites in one or more samples obtained from an individual 108, the concentration or level of one or more components of a sample obtained from an individual 108 that include, but are not limited to, concentrations or levels of: vitamins, minerals, metals, proteins, one or more hormones, hemoglobin, one or more neurotransmitters, metabolites, proteolytic products, antibodies, white blood cells, red blood cells, enzyme activities, lipids, lipoproteins, carbohydrates, phosphates, tumor markers, bacteria, fungi, viruses, parasites, and the like.

At operation 308, the accepting operation 210 may include accepting the data related to one or more performance characteristics of the individual. In some embodiments, one or more accepting units 102 accept data 104 related to one or more performance characteristics of the individual 108.

One or more accepting units 102 may accept data 104 related to numerous performance characteristics of an individual 108. Generally, performance characteristics relate to physical or mental activities in which an individual 108 engages. Examples of such performance characteristics include, but are not limited to, athletic ability, mental ability, sexual ability, ability to interact socially, and the like. For example, in some embodiments, data 104 related to giving a presentation may be accepted.

At operation 310, the accepting operation 210 may include accepting the data related to one or more psychological characteristics of the individual. In some embodiments, one or more accepting units 102 accept data 104 related to one or more psychological characteristics of the individual 108.

One or more accepting units 102 may accept data 104 related to numerous psychological characteristics of an individual 108. Examples of such psychological characteristics include, but are not limited to, the presence or absence of a psychological malady. Examples of psychological maladies include, but are not limited to, antisocial personal disorder, anxiety disorder, avoidant personality disorder, bipolar disorder, conduct disorder, depression, depressive disorder, drug addiction, insomnia, primary sleep disorders, schizophrenia, seasonal affective disorder, sexual disorder, sexual dysfunctions, social anxiety disorder, specific phobia, and the like.

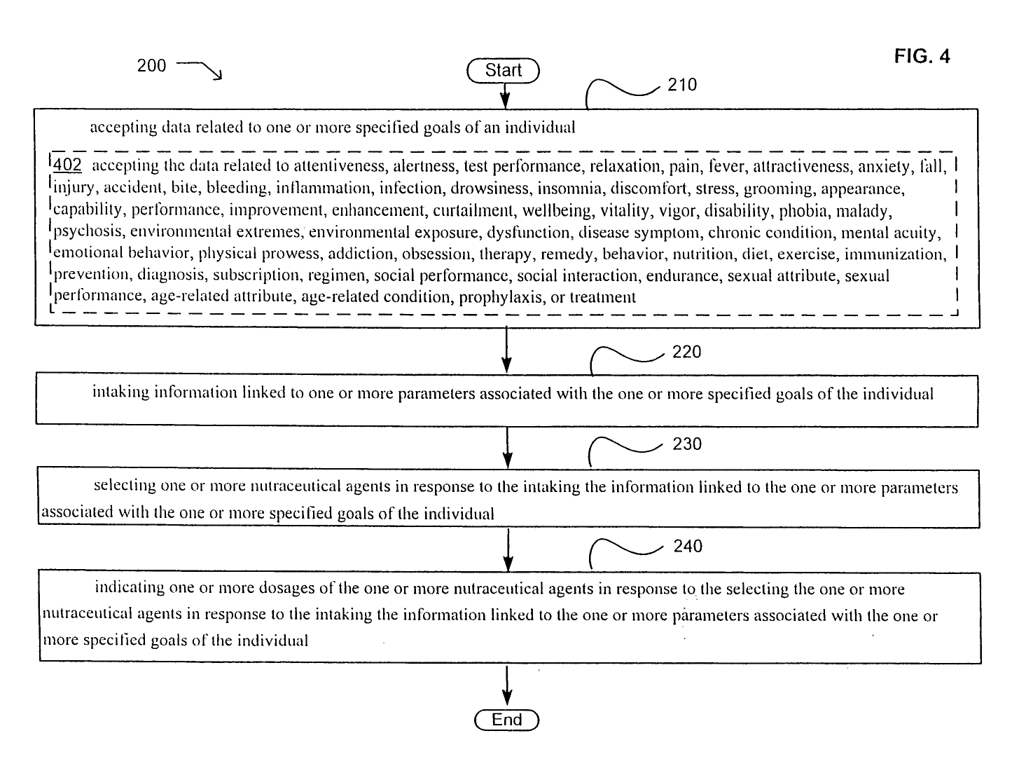

FIG. 4 illustrates alternative embodiments of the example operational flow 200 of FIG. 2. FIG. 4 illustrates example embodiments where the accepting operation 210 may include at least one additional operation 402.

At operation 402, the accepting operation 210 may include accepting the data related to attentiveness, alertness, test performance, relaxation, pain, fever, attractiveness, anxiety, fall, injury, accident, bite, bleeding, inflammation, infection, drowsiness, insomnia, discomfort, stress, grooming, appearance, capability, performance, improvement, enhancement, curtailment, wellbeing, vitality, vigor, disability, phobia, malady, psychosis, environmental extremes, environmental exposure, dysfunction, disease symptom, chronic condition, mental acuity, emotional behavior, physical prowess, addiction, obsession, therapy, remedy, behavior, nutrition, diet, exercise, immunization, prevention, diagnosis, subscription, regimen, social performance, social interaction, endurance, sexual attribute, sexual performance, age-related attribute, age-related condition, prophylaxis, or treatment. In some embodiments, one or more accepting units 102 accept data 104 related to attentiveness, alertness, test performance, relaxation, pain, fever, attractiveness, anxiety, fall, injury, accident, bite, bleeding, inflammation, infection, drowsiness, insomnia, discomfort, stress, grooming, appearance, capability, performance, improvement, enhancement, curtailment, wellbeing, vitality, vigor, disability, phobia, malady, psychosis, environmental extremes, environmental exposure, dysfunction, disease symptom, chronic condition, mental acuity, emotional behavior, physical prowess, addiction, obsession, therapy, remedy, behavior, nutrition, diet, exercise, immunization, prevention, diagnosis, subscription, regimen, social performance, social interaction, endurance, sexual attribute, sexual performance, age-related attribute, age-related condition, prophylaxis, or treatment.

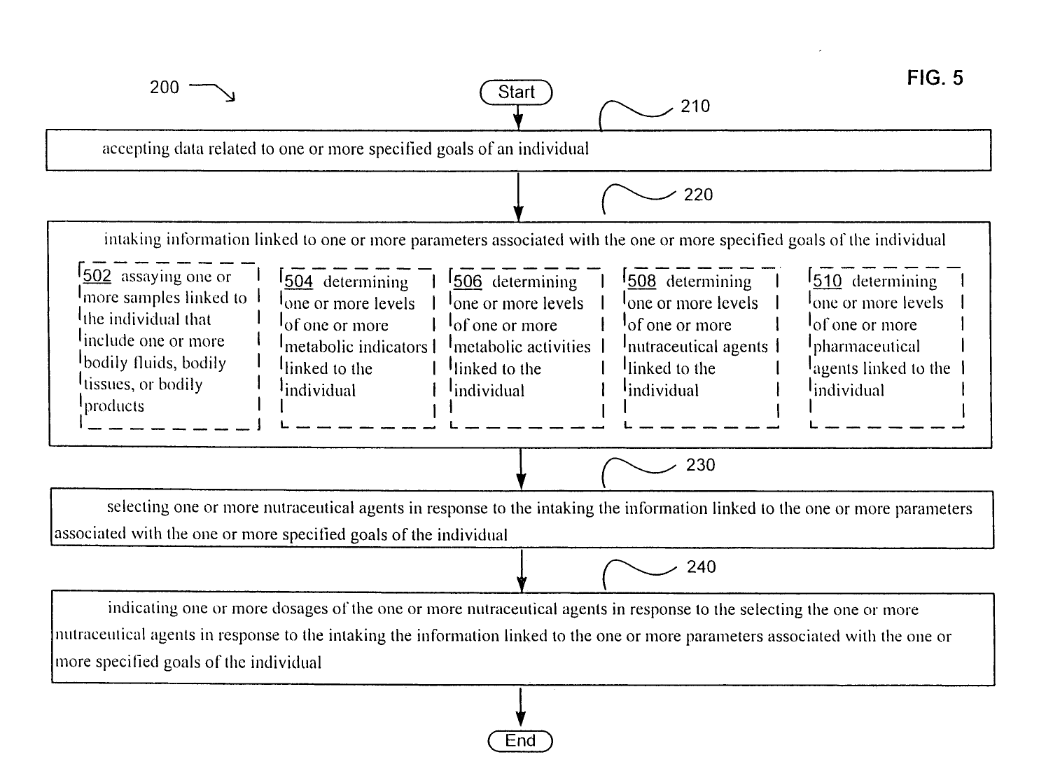

FIG. 5 illustrates alternative embodiments of the example operational flow 200 of FIG. 2. FIG. 5 illustrates example embodiments where the intaking operation 220 may include at least one additional operation. Additional operations may include an operation 502, an operation 504, an operation 506, an operation 508, and/or an operation 510.

At operation 502, the intaking operation 220 may include assaying one or more samples linked to the individual that include one or more bodily fluids, bodily tissues, or bodily products. In some embodiments, one or more intaking units 110 can assay one or more samples linked to the individual 108 that include one or more bodily fluids, bodily tissues, or bodily products. Examples of such samples include, but are not limited to, blood, urine, saliva, synovial fluid, pleural fluid, peritoneal fluid, tears, mucus, ejaculate, skin, muscle, bone, hair, teeth, nails, urine, breath, fecal material, genital products, and the like.

At operation 504, the intaking operation 220 may include determining one or more levels of one or more metabolic indicators linked to the individual. In some embodiments, one or more intaking units 110 can determine one or more levels of one or more metabolic indicators linked to the individual 108.

For example, vitamin B₁₂ and folate are two vitamins that have interdependent roles in nucleic acid synthesis. Deficiencies of either vitamin can cause megaloblastic anemia. Accordingly, in some embodiments, the levels of homocysteine and methylmalonic acid may be determined and used as metabolic indicators to indicate levels of vitamin B₁₂ and folate within an individual 108. In some embodiments, vitamin A deficiency may be assessed by determining albumin levels which are an indirect measure of vitamin A levels. In some embodiments, magnesium levels may be assessed directly. In some embodiments, magnesium levels may be assessed indirectly through analysis of insulin because magnesium deficiency results in impaired insulin secretion. Accordingly, in some embodiments, magnesium replacement may be used to restore insulin secretion. In some embodiments, fluorescent indicators may be used to determine chloride, zinc, and calcium levels as well as pH. Accordingly, numerous metabolic indicators that are linked to an individual 108 may be determined through use of known methods.

At operation 506, the intaking operation 220 may include determining one or more levels of one or more metabolic activities linked to the individual. In some embodiments, one or more intaking units 110 can determine one or more levels of one or more metabolic activities linked to the individual 108 In some embodiments, one or more intaking units 110 can be used to determine one or more enzyme activities of an individual 108. For example, when digested properly, protein supplies acidity to the blood. If an individual 108 is not able to adequately digest protein, the individual's 108 blood acquires excess alkaline reserves which must be continuously dumped via the kidneys into the urine. Accordingly, in some embodiments, blood alkalinity may be tested to assist in determining if an individual 108 is deficient in protease activity. In instances of protease deficiency, an individual 108 may wish to ingest a protease supplement to assist with digestion of protein. In some embodiments, enzyme activity may be determined directly. For example, protease activity can be measured directly through use of peptide substrates having an amino acid sequence that is recognized by a protease to be assayed. Such peptide substrates may be readily prepared or be obtained from commercial sources (i.e., Biotium, Inc., Hayward, Calif.; Biomol International Inc., Plymouth Meeting, Pa.; JPT Peptide Technologies, Inc., Springfield, Va.). In some embodiments, vitamin K deficiency exhibited by an individual 108 may be determined through measurement of Protein Induced by Vitamin K Absence (PIVKA-II) using several known methods (Widdershoven, Clin. Chem., 33(11):2074-2078 (1987)). Examples of such methods include, but are not limited to, electrophoresis-immunofixation and enzyme immunoassay. Numerous metabolic activities of an individual 108 may be determined through use of methods that are known and that have been described (i.e., Clinical Laboratory Medicine, Lippincott Williams and Wilkins, Philadelphia, Second Edition, 2002).

At operation 508, the intaking operation 220 may include determining one or more levels of one or more nutraceutical agents linked to the individual. In some embodiments, one or more intaking units 110 can determine one or more levels of one or more nutraceutical agents 118 linked to the individual 108.

Numerous methods may be used to determine one or more nutraceutical agents 118 that are linked to an individual 108. Examples of such methods include, but are not limited to, chromatographic assay, mass spectrometry, spectrophotometry, immunological assay, and the like. These methods may be performed on numerous types of samples obtained from an individual 108 that are exemplified by bodily fluids, bodily tissues, bodily products and the like.

In some embodiments, one or more intaking units 110 may determine one or more levels of one or more nutraceutical agents 118 that administered to an individual 108 at one or more times. For example, in some embodiments, the level of a nutraceutical agent 118 at the time when administered to an individual 108, at time two hours post-administration, at time four hours post-administration, and at time six hours post-administration may be determined. Accordingly, in some embodiments, the rate at which one or more nutraceutical agents 118 are metabolized by an individual 108 may be determined. In some embodiments, the rate at which a level of a nutraceutical agent 118 is reached within an individual 108 may be determined. Numerous times and concentrations of nutraceutical agents 118 may be transmitted.

At operation 510, the intaking operation 220 may include determining one or more levels of one or more pharmaceutical agents linked to the individual. In some embodiments, one or more intaking units 110 can determine one or more levels of one or more pharmaceutical agents linked to the individual 108.

Numerous methods may be used to determine one or more pharmaceutical agents that are linked to an individual 108. Examples of such methods include, but are not limited to, chromatographic assay, mass spectrometry, spectrophotometry, immunological assay, and the like. These methods may be performed on numerous types of samples obtained from an individual 108 that are exemplified by bodily fluids, bodily tissues, bodily products and the like.

In some embodiments, one or more intaking units 110 may determine one or more levels of one or more pharmaceutical agents that administered to an individual 108 at one or more times. For example, in some embodiments, the level of a pharmaceutical agent at the time when administered to an individual 108, at time two hours post-administration, at time four hours post-administration, and at time six hours post-administration may be determined. Accordingly, in some embodiments, the rate at which one or more pharmaceutical agents are metabolized by an individual 108 may be determined. In some embodiments, the rate at which a level of a pharmaceutical agent is reached within an individual 108 may be determined. Numerous times and concentrations of pharmaceutical agents may be determined.

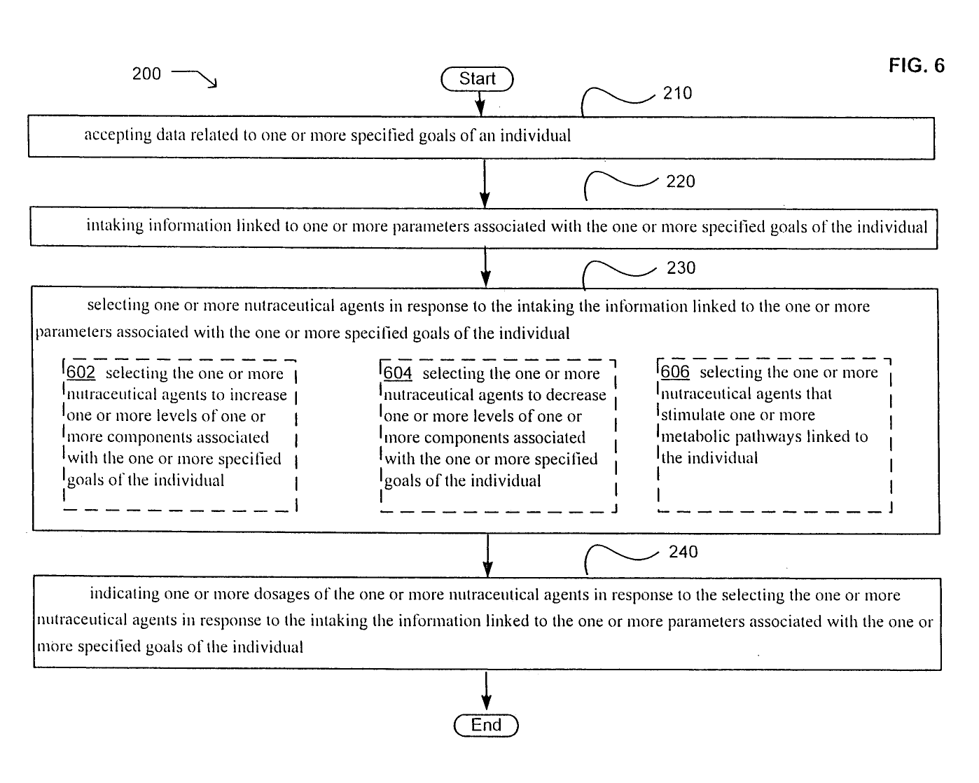

FIG. 6 illustrates alternative embodiments of the example operational flow 200 of FIG. 2. FIG. 6 illustrates example embodiments where the selecting operation 230 may include at least one additional operation. Additional operations may include an operation 602, an operation 604, and/or an operation 606.

At operation 602, the selecting operation 230 may include selecting the one or more nutraceutical agents to increase one or more levels of one or more components associated with the one or more specified goals of the individual. In some embodiments, one or more selecting units 116 can select one or more nutraceutical agents 118 to increase one or more levels of one or more components associated with the one or more specified goals 106 of the individual 108.

In some embodiments, one or more nutraceutical agents 118 may be selected that may directly complement one or more levels of one or more components associated with one or more specified goals 106 of the individual 108. For example, calcium in various forms (i.e., calcium citrate, calcium aspartate, calcium carbonate) may be selected by an individual 108 who wants to avoid or reduce the effects of osteoporosis. In another embodiment, chromium may be selected to lower blood sugar, increase insulin sensitivity, reduce body fat, control hunger, suppress appetite, increase lean body/muscle mass, or substantially any combination thereof. In some embodiments, one or more nutraceutical agents 118 associated with one or more specified goals 106 of an individual 108 may be selected that may be administered to an individual 108 to indirectly supplement a component associated with the one or more specified goals 106. For example, 5-hydroxy-tryptophan may be selected for administration to an individual 108 if the individual 108 suffers from depression and is found to exhibit low levels of serotonin. The 5-hydroxy-tryptophan will be converted to serotonin following administration to the individual 108. Numerous nutraceutical agents 118 may be selected to increase one or more levels of one or more components associated with one or more specified goals 106 of an individual 108.

At operation 604, the selecting operation 230 may include selecting the one or more nutraceutical agents to decrease one or more levels of one or more components associated with the one or more specified goals of the individual. In some embodiments, one or more selecting units 116 can select one or more nutraceutical agents 118 to decrease one or more levels of one or more components associated with the one or more specified goals 106 of the individual 108.

In some embodiments, one or more components may be associated with one or more specified goals 106 of an individual 108. Low density lipoproteins are examples of components that are linked to vascular disease. Accordingly, in some embodiments, one or more nutraceutical agents 118 may be selected that will act to lower the low density lipoprotein concentration of an individual 108. Examples of such nutraceutical agents 118 include, but are not limited to, plant stanols, dietary fiber, stanol esters, isoflavones, diallyl sulfides, niacin, soy protein, and substantially any combination thereof. Glucose is an example of a component that is linked to hyperglycemia. Accordingly, in some embodiments, buckwheat is an example of a nutraceutical agent 118 that may be selected to lower blood glucose levels of an individual 108. Free radical oxygen species that include singlet oxygen, hydroxyl radicals, peroxides, and superoxide radicals are components that act to damage lipids and other cellular structures. Accordingly, one or more nutraceutical agents 118 may be selected to reduce levels of free radicals within an individual 108. Examples of such nutraceutical agents 118 include, but are not limited to, terpenes, carotenoids, limonoids, phenols, flavonoids, isoprenoids, and the like. Numerous other nutraceutical agents 118 may be selected to decrease one or more levels of one or more components associated with the one or more specified goals 106 of the individual 108.

At operation 606, the selecting operation 230 may include selecting the one or more nutraceutical agents that stimulate one or more metabolic pathways linked to the individual. In some embodiments, one or more selecting units 116 can select one or more nutraceutical agents 118 that stimulate one or more metabolic pathways linked to the individual 108.

In some embodiments, stimulation of one or more metabolic pathways may be associated with one or more specified goals 106 of an individual 108. For example, in some embodiments, decline in L-carnitine synthesis is thought to be linked to senile reduction in bone synthesis (Colucci et al., Calcified Tissue International, 76:458-465 (2005)). Dihydroepiandrosterone (DHEA) is thought to affect levels of L-carnitine through promoting the expression of carnitine-synthesizing enzymes (Chiu et al., Calcified Tissue International, 64:527-533 (1999)). Accordingly, DHEA may be selected to stimulate the L-carnitine synthetic pathway to reduce senile reduction in bone synthesis. In other embodiments, buckwheat extract may be selected to lower blood glucose due to the ability of buckwheat extract to activate mitogen activated protein kinase via phospholipase D (Appleton and Lockwood, The Pharmaceutical Journal, 277:78-83 (2006)). In another embodiment, resveratrol has been shown to stimulate endothelial nitric oxide synthase activity (Klinge et al., J. Biol. Chem., 280(9):7460-7468 (2005); Wallerath et al., Circulation, 106(13):1652-1658 (2002)). Endothelial nitric oxide synthase is an enzyme that catalyzes the formation of nitric oxide by vascular endothelial cells. Nitric oxide is needed to maintain arterial relaxation (vasodilation), and impaired nitric oxide-dependent vasodilation is associated with increased risk of cardiovascular disease (Duffy and Vita, Curr. Opin. Lipidol., 14(1):21-27 (2003)). Accordingly, in some embodiments, resveratrol may be selected to reduce cardiovascular disease. Numerous nutraceutical agents 18 may be selected to one or more metabolic pathways linked to the individual 108.

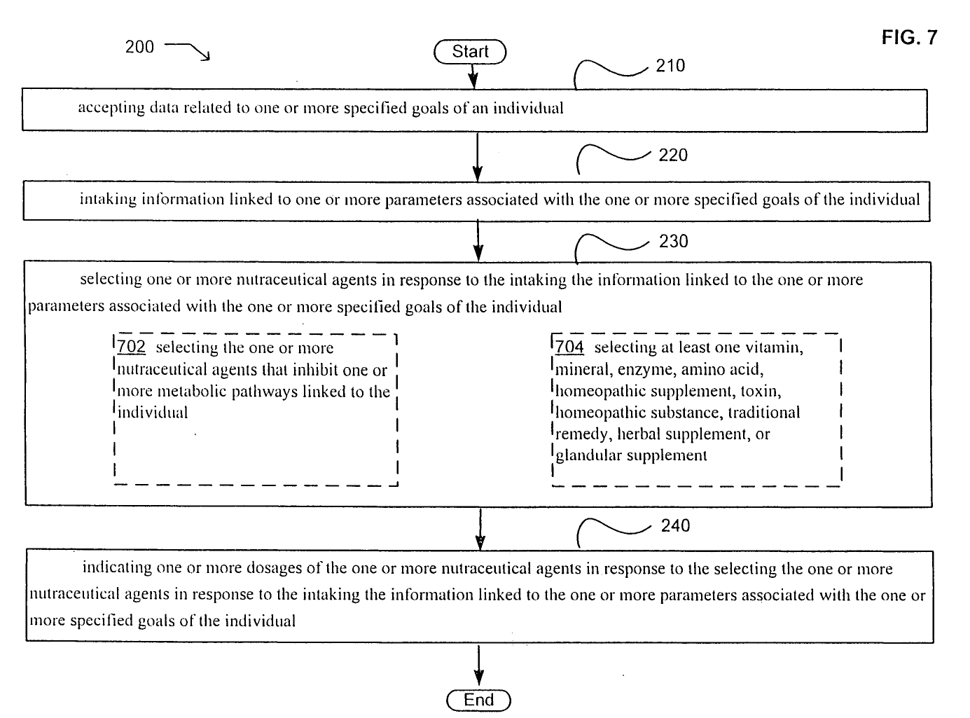

FIG. 7 illustrates alternative embodiments of the example operational flow 200 of FIG. 2. FIG. 7 illustrates example embodiments where the selecting operation 230 may include at least one additional operation. Additional operations may include an operation 702, and/or an operation 704.

At operation 702, the selecting operation 230 may include selecting the one or more nutraceutical agents that inhibit one or more metabolic pathways linked to the individual. In some embodiments, one or more selecting units 116 can select one or more nutraceutical agents 18 that inhibit one or more metabolic pathways linked to the individual 108.

In some embodiments, one or more metabolic pathways may be inhibited through inhibition of one or more enzymes that participate within the metabolic pathway. Such metabolic pathways may be involved with a specified goal 106 of an individual 108. For example, angiotensin-converting enzyme has been linked to high blood pressure. Flavonoids have been shown to inhibit angiotensin-converting enzyme (Actis-Goretta et al., J. Agric. Food Chem., 54(1):229-234 (2006)). Accordingly, flavonoids may be selected to assist in the reduction of blood pressure (Li et al., Chin. J. Physiol., 48(2):101-106 (2005); Machha and Mustafa, J. Cardiovasc. Pharmacol., 46(1):36-40 (2005)). Genistein, one of the predominant soy isoflavones, has been shown to compete with 17beta-estradiol for estrogen receptor binding because of its structural similarity, resulting in agonistic or antagonistic activity. This has been shown to cause inhibition of cell growth in breast and prostate cancers in vivo and in vitro. Accordingly, soy isoflavones containing genistein may be selected as a nutraceutical agent 118 for cancer chemoprevention (Sarkar et al., Mini Rev. Med. Chem., 6(4):401-407 (2006)). The isoflavonoids, daidzin, daidzein, and puerarin have been shown to reduce alcohol consumption (Lin RC, Alcohol Clin. Exp. Res., 20(4):659-663 (1996)). A link between daidzin's capacity to reduce alcohol consumption and its ability to increase the liver mitochondrial monoamine oxidase: aldehyde dehydrogenase activity ratio has been established (Keung, Med. Res. Rev., 23(6):669-696 (2003)). This increase in ratio is thought to occur through inhibition of aldehyde dehydrogenase activity. Accordingly, isoflavonoid mixtures that include daidzin, daidzein, and/or puerarin may be selected to lower alcohol consumption. Numerous other nutraceutical agents 118 may be selected that inhibit one or more metabolic pathways linked to the individual 108.

At operation 704, the selecting operation 230 may include selecting at least one vitamin, mineral, enzyme, amino acid, homeopathic supplement, toxin, homeopathic substance, traditional remedy, herbal supplement, or glandular supplement. In some embodiments, one or more selecting units 116 can select at least one vitamin, mineral, enzyme, amino acid, homeopathic supplement, toxin, homeopathic substance, traditional remedy, herbal supplement, or glandular supplement.

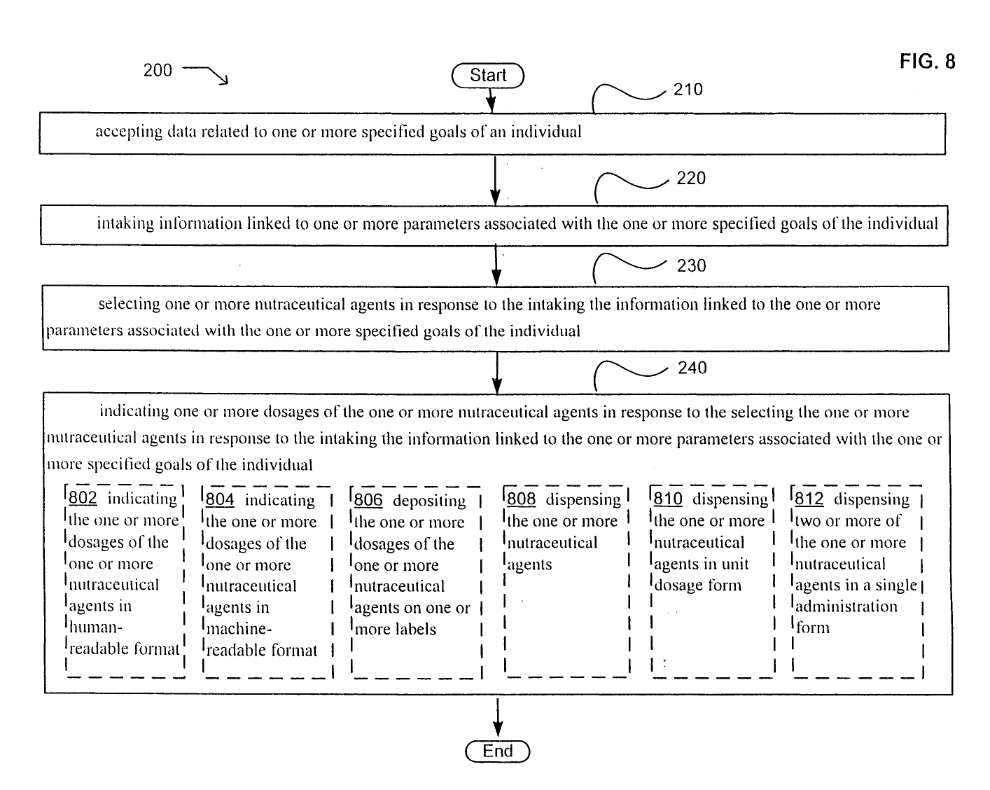

FIG. 8 illustrates alternative embodiments of the example operational flow 200 of FIG. 2. FIG. 8 illustrates example embodiments where the indicating operation 240 may include at least one additional operation. Additional operations may include an operation 802. an operation 804, an operation 806, an operation 808, an operation 810, and/or an operation 812.

At operation 802, the indicating operation 240 may include indicating the one or more dosages of the one or more nutraceutical agents in human-readable format. In some embodiments, one or more indicating units 120 can indicate the one or more dosages 122 of the one or more nutraceutical agents 118 in human-readable format.

In some embodiments, one or more indicating units 120 may indicate one or more dosages 122 of one or more nutraceutical agents 118 on a visual display, a touch display, an audible display, and the like. For example, in some embodiments, one or more indicating units 120 may display one or more dosages 122 on a light emitting diode display, a liquid crystal display, various monitors, and the like. Such visual displays may indicate one or more dosages 122 through display of colors, pictures, printed language, and the like. In some embodiments, one or more indicating units 120 may display one or more dosages 122 on a touch display (i.e., a touch pad display in Braille for use by blind or visually impaired persons). In some embodiments, one or more indicating units 120 may display one or more dosages 122 through use of an audible display that verbally speaks to an individual 108.

In some embodiments, one or more indicating units 120 may display one or more dosages 122 of one or more nutraceutical agents 118 in typographical symbols in numerous languages that are in human-readable format. In some embodiments, one or more indicating units 120 may display one or more dosages 122 of one or more nutraceutical agents 118 and the formulation type of the one or more nutraceutical agents 118 in typographical symbols in numerous languages that are in human-readable format. In some embodiments, one or more indicating units 120 may display one or more dosages 122 of one or more nutraceutical agents 118 in pictographic form that is in human-readable format. In some embodiments, one or more indicating units 120 may display one or more dosages 122 of one or more nutraceutical agents 18 and the formulation type of the one or more nutraceutical agents 118 in pictographic form that is in human-readable format. In some embodiments, one or more indicating units 120 may display one or more dosages 122 of one or more nutraceutical agents 118 according to a color scheme that is in human-readable format. In some embodiments, one or more indicating units 120 may display one or more dosages 122 of one or more nutraceutical agents 118 and the formulation type of the one or more nutraceutical agents 118 according to a color scheme that is in human-readable format. In some embodiments, one or more indicating units 120 may display one or more dosages 122 of one or more nutraceutical agents 118, one or more routes of administration for the one or more nutraceutical agents 118, one or more times and/or periodicities for administration of the one or more nutraceutical agents 118, one or more formulation types for the one or more nutraceutical agents 118, whether to administer the one or more nutraceutical agents 118 with or without food, whether to administer the one or more nutraceutical agents 118 with or without other nutraceutical aoents 118 and/or pharmaceutical agents, and substantially any combination thereof. Dosages 122 may be displayed according to numerous methods that are known and have been described (i.e., U.S. patent application Ser. No. 11/474,109, entitled CUSTOMIZED VISUAL MARKING FOR MEDICATION LABELING, filed 23 Jun. 2006, herein incorporated by reference to the extent such subject matter is not inconsistent herewith). In some embodiments, one or more indicating units 120 may indicate one or more dosages 122 in human-readable format and machine-readable format.

At operation 804, the indicating operation 240 may include indicating the one or more dosages of the one or more nutraceutical agents in machine-readable format. In some embodiments, one or more indicating units 120 can indicate the one or more dosages 122 of the one or more nutraceutical agents 118 in machine-readable format.

In some embodiments, one or more indicating units 120 mav indicate one or more dosages 122 of one or more nutraceutical agents 118 in machine-readable format. Numerous methods may be used to indicate one or more dosages 122. Examples of such methods include, but are not limited to, radio frequency identification, bar coding, typographical methods, symbol based methods (i.e., use of symbols that represent dosages 122 and nutraceutical agents 118), optical methods (i.e., pulsed light), and the like.

In some embodiments, one or more indicating units 120 may indicate one or more dosages 122 of one or more nutraceutical agents 118 and the formulation type of the one or more nutraceutical agents 118 in machine-readable form. In some embodiments, one or more indicating units 120 may indicate one or more dosages 122 of one or more nutraceutical agents 118 in a pictographic form that is in machine-readable format. In some embodiments, one or more indicating units 120 may indicate one or more dosages 122 of one or more nutraceutical agents 118 and the formulation type of the one or more nutraceutical agents 118 in a pictographic form that is in machine-readable format. In some embodiments, one or more indicating units 120 may indicate one or more dosages 122 of one or more nutraceutical agents 118 according to a color scheme that is in machine-readable format. In some embodiments, one or more indicating units 120 may indicate one or more dosages 122 of one or more nutraceutical agents 118 and the formulation type of the one or more nutraceutical agents 118 according to a color scheme that is in machine-readable format. In some embodiments, one or more indicating units 120 may indicate one or more dosages 122 of one or more nutraceutical agents 118, one or more routes of administration for the one or more nutraceutical agents 118., one or more times and/or periodicities for administration of the one or more nutraceutical agents 118, one or more formulation types for the one or more nutraceutical agents 118, whether to administer the one or more nutraceutical agents 118 with or without food, whether to administer the one or more nutraceutical agents 118 with or without other nutraceutical agents 118 and/or pharmaceutical agents, and substantially any combination thereof. Numerous labeling methods are known and have been described that may be adapted into a machine-readable format (i.e., U.S. patent application Ser. No. 11/474,109, entitled CUSTOMIZED VISUAL MARKING FOR MEDICATION LABELING, filed 23, Jun. 2006, herein incorporated by reference to the extent such subject matter is not inconsistent herewith). In some embodiments, one or more indicating units 120 may indicate one or more dosages 122 in human-readable and in a machine-readable format.

At operation 806, the indicating operation 240 may include depositing the one or more dosages of the one or more nutraceutical agents on one or more labels. In some embodiments, one or more indicating units 120 can deposit the one or more dosages 122 of the one or more nutraceutical agents 118 on one or more labels.

In some embodiments, one or more indicating units 120 may print one or more labels that include one or more dosages 122 of one or more nutraceutical agents 118 and the formulation type of the one or more nutraceutical agents 118. In some embodiments, one or more indicating units 120 may print one or more labels that include one or more dosages 122 of one or more nutraceutical agents 118 in pictographic form. In some embodiments, one or more indicating units 120 may print one or more labels that include one or more dosages 122 of one or more nutraceutical agents 118 and the formulation type of the one or more nutraceutical agents 118 in pictographic form. In some embodiments, one or more indicating units 120 may print one or more labels that include one or more dosages 122 of one or more nutraceutical agents 118 according to a color scheme. In some embodiments, one or more indicating units 120 may print one or more labels that include one or more dosages 122 of one or more nutraceutical agents 118 and the formulation type of the one or more nutraceutical agents 118 according to a color scheme. In some embodiments, one or more indicating units 120 may indicate one or more dosages 122 of one or more nutraceutical agents 118, one or more routes of administration for the one or more nutraceutical agents 118, one or more times and/or periodicities for administration of the one or more nutraceutical agents 118, one or more formulation types for the one or more nutraceutical agents 118, whether to administer the one or more nutraceutical agents 118 with or without food, whether to administer the one or more nutraceutical agents 118 with or without other nutraceutical agents 118 and/or pharmaceutical agents, and substantially any combination thereof. Numerous labeling methods are known and have been described which may be adapted into machine-readable form (i.e., U.S. patent application Ser. No. 11/474,109, entitled CUSTOMIZED VISUAL MARKING FOR MEDICATION LABELING, filed 23 Jun. 2006, herein incorporated by reference to the extent such subject matter is not inconsistent herewith).

In some embodiments, one or more indicating units 120 may indicate one or more dosages 122 in human-readable and machine-readable form. Numerous types of depositing methods may be used by one or more indicating units 120. Examples of such methods include, but are not limited to, printing methods (i.e., stamping, ink jet printing, laser printing, and the like). In some embodiments, ink containing magnetic particles may be used.

At operation 808, the indicating operation 240 may include dispensing the one or more nutraceutical agents. In some embodiments, one or more indicating units 120 can dispense the one or more nutraceutical agents 118.

One or more indicating units 120 may dispense one or more nutraceutical agents 118 in numerous dosage forms. In some embodiments, one or more indicating units 120 may dispense one or more nutraceutical agents 118 in powder form. In some embodiments, one or more indicating units 120 may dispense one or more nutraceutical agents 118 in liquid form. In some embodiments, one or more indicating units 120 may dispense one or more nutraceutical agents 118 in tablet form. In some embodiments, one or more indicating units 120 may dispense one or more nutraceutical agents 118 in capsule form.

At operation 810, the indicating operation 240 may include dispensing the one or more nutraceutical agents in unit dosage form. In some embodiments, one or more indicating units 120 can dispense the one or more nutraceutical agents 118 in unit dosage form.

In some embodiments, one or more indicating units 120 may dispense one or more nutraceutical agents 118 in a unit dosage form in which the entire dosage 122 is to be administered to an individual 108 as a single dosage 122. In some embodiments, one or more indicating units 120 may dispense one or more nutraceutical agents 118 in a unit dosage form that may be administered such that the individual 108 will maintain a level of the one or more nutraceuticals for a given time period. For example, in some embodiments, a unit dosage form may be an amount of one or more nutraceutical agents 118 that will allow the one or more nutraceutical agents 118 to be maintained within an individual 108 for four hours. Numerous criteria mav be used to determine a unit dosage form. Examples of such criteria include, but are not limited to, physical characteristics of the individual 108, physiological characteristics of the individual 108, activity of the individual 108, and the like.

At operation 812, the indicating operation 240 may include dispensing two or more of the one or more nutraceutical agents in a single administration form. In some embodiments, one or more indicating units 120 can dispense two or more of the one or more nutraceutical agents 118 in a single administration form.

In some embodiments, one or more indicating units 120 may dispense two or more nutraceutical agents 118 in a single administration form to provide for administration of the two or more nutraceutical agents 118 to an individual 108. Such methods have been described (i.e., U.S. patent application Ser. No. 11/453,571, filed 14 Jun. 2006; U.S. patent application Ser. No. 11/478,341, filed 28 Jun. 2006; U.S. patent application Ser. No. 11/478,296, filed 28 Jun. 2006; and U.S. patent application No. 11/486,998, filed 14 Jul. 2006, herein incorporated by reference to the extent such subject matter is not inconsistent herewith).

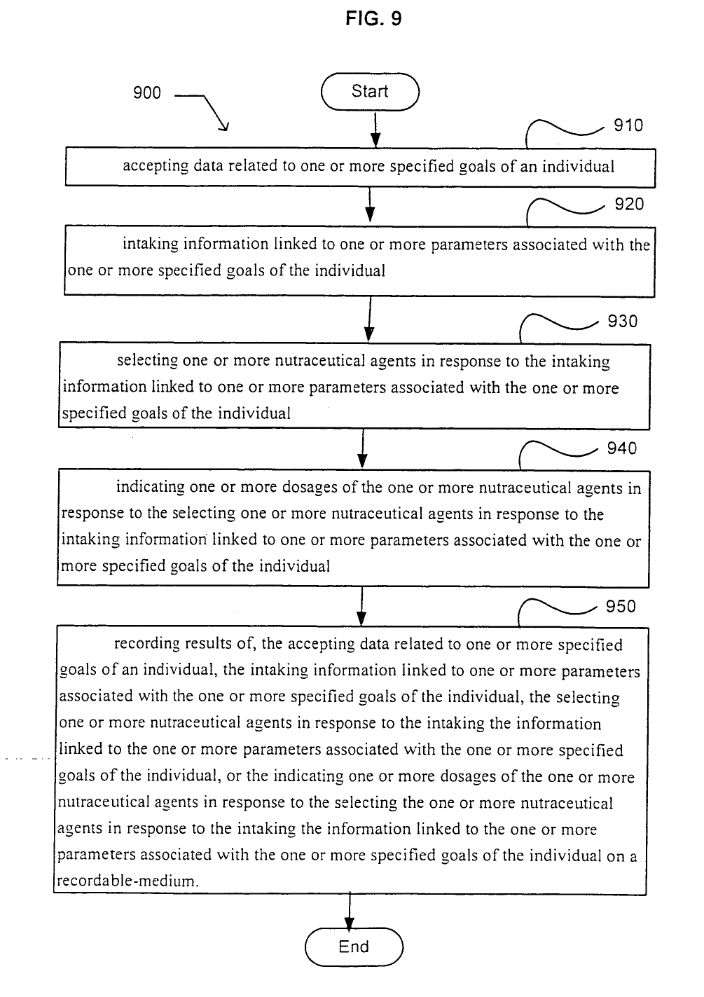

FIG. 9 illustrates an operational flow 900 that includes an accepting operation 910, an intaking operation 920, a selecting operation 930, and an indicating operation 940 (which correspond to the accepting operation 210, the intaking operation 220, the selecting operation 230, and the indicating operation 240 illustrated in FIG. 2) with an additional recording operation 950. In FIG. 9, discussion and explanation may be provided with respect to the above-described example of FIG. 1, and/or with respect to other examples and contexts. However, it should be understood that the operations may be executed in a number of other environments and contexts, and/or modified versions of FIG. 1. Also, although the various operations are presented in the sequence(s) illustrated, it should be understood that the various operations may be performed in other orders than those which are illustrated, or may be performed concurrently.

After a start operation, the operational flow 900 includes the operations 910, 920, 930, and 940 (which correspond to operations 210, 220, 230, and 240 as described with regard to FIG. 2) and an additional recording operation 950 involving recording results of, the accepting data related to one or more specified goals of an individual, the intaking information linked to one or more parameters associated with the one or more specified coals of the individual, the selecting one or more nutraceutical agents in response to the intaking the information linked to the one or more parameters associated with the one or more specified goals of the individual, or the indicating one or more dosages of the one or more nutraceutical agents in response to the selecting the one or more nutraceutical agents in response to the intaking the information linked to the one or more parameters associated with the one or more specified goals of the individual on a recordable-medium. In some embodiments, one or more recording units 124 may record results of, the accepting data 104 related to one or more specified goals 106 of an individual 108, the intaking information 112 linked to one or more parameters 114 associated with the one or more specified goals 106 of the individual 108, the selecting one or more nutraceutical agents 118 in response to the intaking information 112 linked to one or more parameters 114 associated with the one or more specified goals 106 of the individual 108, or the indicating one or more dosages 122 of the one or more nutraceutical agents 118 in response to the selecting one or more nutraceutical agents 118 in response to the intaking information 112 linked to one or more parameters 114 associated with the one or more specified goals 106 of the individual 108 on a recordable-medium 126.

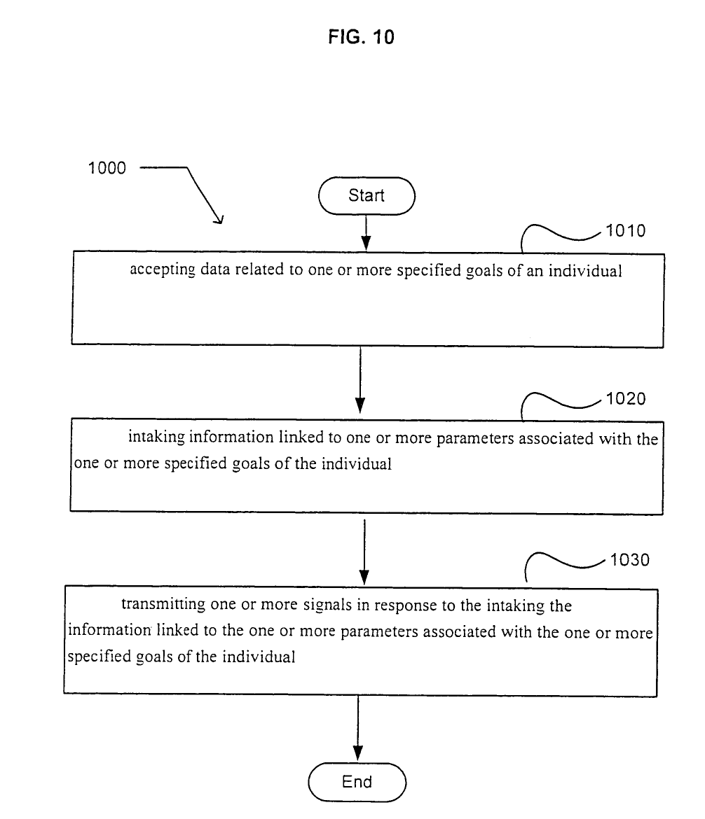

FIG. 10 illustrates an operational flow 1000 representing examples of operations that are related to the performance of a method for nutraceutical agent 118 and dosing. In FIG. 10 and in following figures that include various examples of operations used during performance of the method, discussion and explanation may be provided with respect to the above-described example of FIG. 1, and/or with respect to other examples and contexts. However, it should be understood that the operations may be executed in a number of other environments and contexts. and/or modified versions of FIG. 1. Also, although the various operations are presented in the sequence(s) illustrated, it should be understood that the various operations may be performed in other orders than those which are illustrated, or may be performed concurrently.

After a start operation, the operational flow 1000 includes an accepting operation 1010 involving accepting data related to one or more specified goals of an individual. In some embodiments, one or more accepting units 102 may accept data 104 related to one or more specified goals 106 of an individual 108.

In some embodiments, one or more accepting units 102 may accept data 104 related to one or more specified goals 106 of an individual 108. The one or more specified goals 106 may be virtually any goal to be achieved, or attempted by, an individual 108 that may be affected by administration of one or more nutraceutical agents 118 to the individual 108. In some embodiments, administration of one or more nutraceutical agents 118 to an individual 108 may assist the individual 108 to achieve one or more specified goals 106. In some embodiments, administration of one or more nutraceutical agents 118 to an individual 108 may stimulate the individual 108 to achieve one or more specified goals 106. In some embodiments, administration of one or more nutraceutical agents 118 to an individual 108 may reduce an inhibition coupled to the individual 108 to promote achieving one or more specified goals 106 by the individual 108. Numerous examples of specified goals 106 of an individual 108 exist. In some embodiments, specified goals 106 of an individual 108 may be related to attentiveness, alertness, test performance, relaxation, pain, fever, attractiveness, anxiety, fall, injury, accident, bite, bleeding, inflammation, infection, drowsiness, insomnia, discomfort, stress, grooming, appearance, capability, performance, improvement, enhancement, curtailment, wellbeing, vitality, vigor, disability, phobia, malady, psychosis, environmental extremes, environmental exposure, dysfunction, disease symptom, chronic condition, mental acuity, emotional behavior, physical prowess, addiction, obsession, therapy, remedy, behavior, nutrition, diet, exercise, immunization, prevention, diagnosis, subscription, regimen, social performance, social interaction, endurance, sexual attribute, sexual performance, age-related attribute, age-related condition, prophylaxis, treatment, and substantially any combination thereof. In some embodiments, data 104 may include identification of one or more of the specified goals 106 of an individual 108. In some embodiments, data 104 may include characteristics of an individual 108. Examples of such data 104 may include, but are not limited to, physical characteristics, metabolic characteristics, financial characteristics, and the like. In some embodiments, data 104 may include, an individual's 108 height, weight, gender, kidney function, liver function, level of physical fitness, age, allergic response, metabolic level (i.e., resting metabolic rate and/or activity-related metabolic rate), disease state, body fat percentage, body mass index, personal health habits (i.e., smoking, alcohol consumption, diet, illegal drug use, and the like), family health history, insurance coverage, food supplement usage, nutraceutical agent 118 usage, non-prescription drug use, prescription drug use, pregnancy status, and the like. In some embodiments, the one or more characteristics may be specifically associated with an individual 108. As such, in some embodiments, the one or more characteristics may be unique to the individual 108 as opposed to being common to a group. For example, in some embodiments, an individual 108 may be a member of a group of persons who are diabetic while exhibiting one or more characteristics, such as metabolic characteristics, that are unique to the individual 108. Accordingly, in some embodiments, data 104 may be input that provides for selection of nutraceutical agents 118 in accordance with one or more characteristics and specified goals 106 of an individual 108.

After a start operation, the operational flow 1000 includes an intaking operation 1020 involving intaking information linked to one or more parameters associated with the one or more specified goals of the individual. In some embodiments, one or more intaking units 110 mav intake information 112 linked to one or more parameters 114 associated with the one or more specified goals 106 of the individual 108.

In some embodiments, one or more intaking units 110 may intake information 112 linked to one or more parameters 114 associated with the one or more specified goals 106 of the individual 108. Numerous parameters 114 may be associated with one or more specified goals 106 of an individual 108. Examples of such parameters 114 include, but are not limited to, blood sugar levels, blood insulin levels, blood iron levels, hormone levels (amine-derived hormones: such as catecholamines (adrenaline, dopamine, noradrenaline); tryptophan derivatives (melatonin, serotonin); tyrosine derivatives (thyroxine and triiodothyronine); peptide hormones such as antimullerian hormone, adiponectin, adrenocorticotropic hormone, angiotensinogen and angiotensin, antidiuretic hormone, atrial-natriuretic peptide, calcitonin, cholecystokinin, corticotropin-releasing hormone, erytiropoietin, follicle-stimulating hormone, gastrin, ghrelin, glucagons, gonadotropin-releasing hormone, growth hormone-releasing hormone, human chorionic gonadotropin, growth hormone, inhibin, insulin, insulin-like growth factor, luteinizing hormone, melanocyte stimulating hormone, neuropeptide Y, oxytocin, parathyroid hormone, prolactin, relaxin, secretin, somatostatin, thrombopoietin, thyroid-stimulating hormone, thyrotropin-releasing hormone; steroid hormones: Glucocorticoids (cortisol); Mineralocorticoids (aldosterone); sex steroids: androgens (testosterone, dehydroepiandrosterone, dehydroepiandrosterone sulfate, androstenedione, dihydrotestosterone); estrogens (estradiol); progestagens (progesterone and progestins); sterol hormones: vitamin D derivatives (calcitriol); lipid hormones (prostaglandins, leukotrienes, prostacyclin, and thromboxane)), nutraceutical agent 118 levels, pharmaceutical agent levels, cytokine levels, and the like (i.e., Fitzgerald et al., The Cytokine Facts Book, Academic Press, San Francisco, Second Edition, (2001)). Methods to gain information 112 with regard to components of biological systems are known (i.e., Clinical Laboratory Medicine, Lippincott Williams and Wilkins, Philadelphia, Second Edition, 2002). In some embodiments, one or more intaking units 110 may include instrumentation that provides for analysis of a sample obtained from an individual 108. For example, in some embodiments, an intaking unit 110 may be configured to intake a blood sample obtained from an individual 108 and analyze the blood sample to determine one or more parameters 114 associated with one or more specified goals 106 of an individual 108 (i.e., determine the level of free testosterone or the level of melatonin in a blood sample obtained from an individual 108). Numerous analytical technologies are known and may be included within one or more intakin a units 110. Examples of such technologies include, but are not limited to, gas chromatography, mass spectrometry, atomic absorption, immunoassay based methods, microfluidic based methods, spectrophotometry (i.e., infrared, ultraviolet, fluorescence, and the like), surface plasmon resonance, fluorescence resonance energy transfer, and the like. In some embodiments, one or more intakin o units 110 may intake information 112 linked to one or more parameters 114 associated with the one or more specified goals 106 of an individual 108 from a source that is independent of the one or more intaking units 110. In some embodiments, one or more intaking units 110 may intake information 112 linked to one or more parameters 114 associated with the one or more specified goals 106 of an individual 108 from a source that is coupled to the one or more intaking units 110.

After a start operation, the operational flow 1000 includes a transmitting operation 1030 involving transmitting one or more signals in response to the intaking the information linked to the one or more parameters associated with the one or more specified goals of the individual. In some embodiments, one or more transmitting units 128 may transmit one or more signals 130 in response to the intaking information 112 linked to one or more parameters 114 associated with the one or more specified goals 106 of the individual 108.

In some embodiments, one or more transmitting units 128 transmit one or more levels of one or more metabolic indicators linked to the individual 108. In some embodiments, one or more transmitting units 128 transmit one or more levels of one or more metabolic activities linked to the individual 108. In some embodiments, one or more transmitting units 128 transmit one or more levels of one or more nutraceutical agents 118 linked to the individual 108. In some embodiments, one or more transmitting units 128 transmit one or more levels of one or more pharmaceutical agents linked to the individual 108. In some embodiments, one or more transmitting units 128 transmit one or more parameters 114 associated with the one or more specified goals 106 of the individual 108. In some embodiments, one or more transmitting units 128 transmit one or more instructions to select one or more nutraceutical agents 118 in response to the intaking information 112 linked to one or more parameters 114 associated with the one or more specified goals 106 of the individual 108. In some embodiments, one or more transmitting units 128 transmit one or more signals 130 to select one or more nutraceutical agents 118 to increase one or more levels of one or more components associated with the one or more specified goals 106 of the individual 108. In some embodiments, one or more transmitting units 128 transmit one or more signals 130 to select one or more nutraceutical agents 118 to decrease one or more levels of one or more components associated with the one or more specified goals 106 of the individual 108. In some embodiments, one or more transmitting units 128 transmit one or more signals 130 to select one or more nutraceutical agents 118 that stimulate one or more metabolic pathways linked to the individual 108. In some embodiments, one or more transmitting units 128 transmit one or more signals 130 to select one or more nutraceutical agents 118 that inhibit one or more metabolic pathways linked to the individual 108. In some embodiments, one or more transmitting units 128 transmit one or more signals 130 to select at least one vitamin, mineral, enzyme, amino acid, homeopathic supplement, toxin, homeopathic substance, traditional remedy, herbal supplement, glandular supplement, or substantially any combination thereof.

In some embodiments, one or more transmitting units 128 may transmit one or more signals 130 that include data 104 related to one or more specified goals 106 of an individual 108. One or more transmitting units 128 may transmit one or more signals 130 through use of numerous technologies. Examples of such technologies include, but are not limited to, wireless transmission, telephone, internet transmission, digital transmission, analog transmission, optical transmission, and the like.

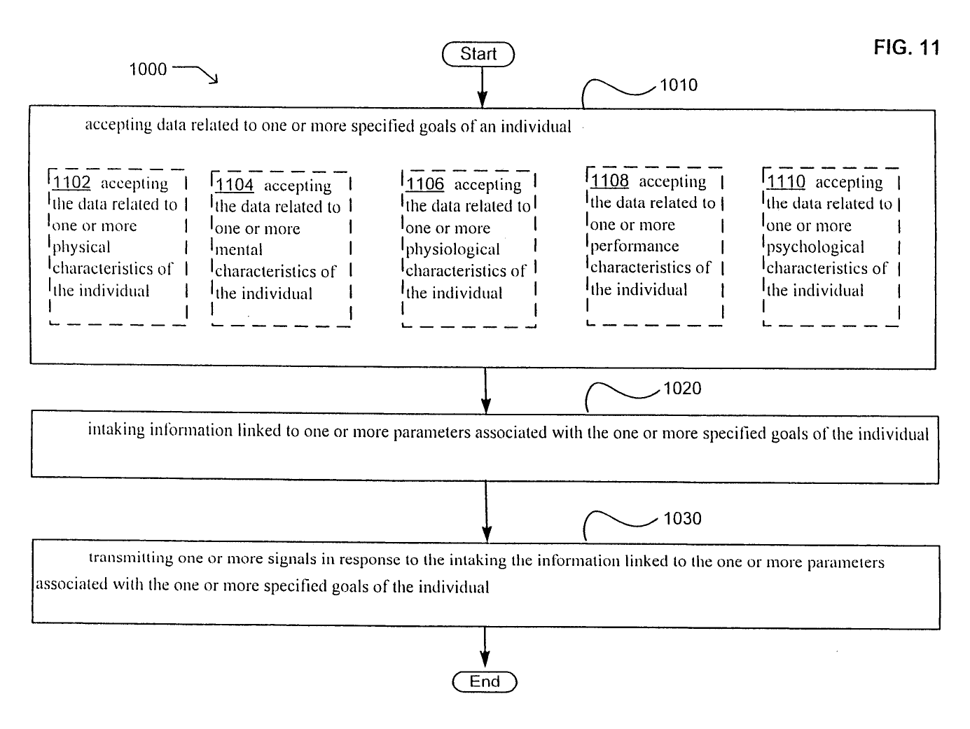

FIG. 11 illustrates alternative embodiments of the example operational flow 1000 of FIG. 10. FIG. 11 illustrates example embodiments where the accepting operation 1010 may include at least one additional operation. Additional operations may include an operation 1102, an operation 1104, an operation 1106, an operation 1108, and/or an operation 1110.

At operation 1102, the accepting operation 1010 may include accepting the data related to one or more physical characteristics of the individual. In some embodiments, one or more accepting units 102 accept data 104 related to one or more physical characteristics of the individual 108.

One or more accepting units 102 may accept data 104 related to numerous physical characteristics of an individual 108. Examples of such physical characteristics include, but are not limited to, height, weight, gender, kidney function, liver function, level of physical fitness, age, allergic response, metabolic level (i.e., resting metabolic rate and/or activity-related metabolic rate), disease state, body fat percentage, body mass index, personal health habits (i.e., smoking, alcohol consumption, diet, illegal drug use, and the like), family health history, food supplement usage, nutraceutical agent 118 usage, non-prescription drug use, prescription drug use, pregnancy status, and the like. In some embodiments, such data 104 may be used to identify one or more nutraceutical agents 118 that are compatible with an individual 108. For example, if an individual 108 is taking a selective serotonin reuptake inhibitor (SSRI), one or more nutraceutical agents 118 should not be selected that would interfere with the serotonin reuptake inhibitor.

At operation 1104, the accepting operation 1010 may include accepting the data related to one or more mental characteristics of the individual. In some embodiments, one or more accepting units 102 accept data 104 related to one or more mental characteristics of the individual 108.

One or more accepting units 102 may accept data 104 related to numerous mental characteristics of an individual 108. Examples of such mental characteristics include, but are not limited to, daily time periods when the mental processes of an individual 108 are most acute or least acute (i.e., morning, afternoon, evening, night), an individual's 108 sleep schedule, an individual's 108 daily schedule of activities (i.e., meetings, presentations, travel, athletic activity), and the like.

At operation 1106, the accepting operation 1010 may include accepting the data related to one or more physiological characteristics of the individual. In some embodiments, one or more accepting units 102 accept data 104 related to one or more physiological characteristics of the individual 108.

One or more accepting units 102 may accept data 104 related to numerous physiological characteristics of an individual 108. Examples of such physiological characteristics include, but are not limited to, the ability of an individual 108 to metabolize one or more nutraceutical agents 118, the ability of an individual 108 to metabolize one or more pharmaceutical agents, the response of an individual 108 to one or more pharmaceutical agents, the response of an individual 108 to one or more nutraceutical agents 118, the concentration or level of one or more metabolites in one or more samples obtained from an individual 108, the concentration or level of one or more components of a sample obtained from an individual 108 that include, but are not limited to, concentrations or levels of: vitamins, minerals, metals, proteins, one or more hormones, hemoglobin, one or more neurotransmitters, metabolites, proteolytic products, antibodies, white blood cells, red blood cells, enzyme activities, lipids, lipoproteins, carbohydrates, phosphates, tumor markers, bacteria, fungi, viruses, parasites, and the like.

At operation 1108, the accepting operation 1010 may include accepting the data related to one or more performance characteristics of the individual. In some embodiments, one or more accepting units 102 accept data 104 related to one or more performance characteristics of the individual 108.

One or more accepting units 102 may accept data 104 related to numerous performance characteristics of an individual 108. Generally, performance characteristics relate to physical or mental activities in which an individual 108 engages. Examples of such performance characteristics include, but are not limited to, athletic ability, mental ability, sexual ability, ability to interact socially, and the like. For example, in some embodiments, data 104 related to giving a presentation may be accepted.

At operation 1110, the accepting operation 1010 may include accepting the data related to one or more psychological characteristics of the individual. In some embodiments, one or more accepting units 102 accept data 104 related to one or more psychological characteristics of the individual 108.

One or more accepting units 102 may accept data 104 related to numerous psychological characteristics of an individual 108. Examples of such psychological characteristics include, but are not limited to, the presence or absence of a psychological malady. Examples of psychological maladies include, but are not limited to, antisocial personal disorder, anxiety disorder, avoidant personality disorder, bipolar disorder, conduct disorder, depression, depressive disorder, drug addiction, insomnia, primary sleep disorders, schizophrenia, seasonal affective disorder, sexual disorder, sexual dysfunctions, social anxiety disorder, specific phobia, and the like.

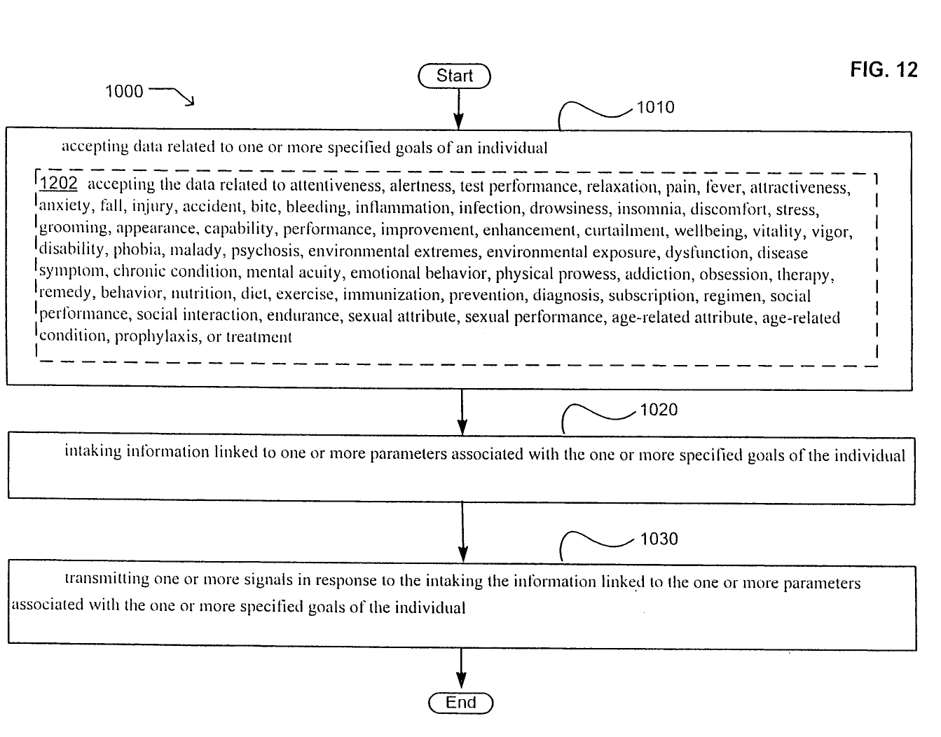

FIG. 12 illustrates alternative embodiments of the example operational flow 1000 of FIG. 10. FIG. 12 illustrates example embodiments where the accepting operation 1010 may include at least one additional operation 1202.

At operation 1202, the accepting operation 1010 may include accepting the data related to attentiveness, alertness, test performance, relaxation, pain, fever, attractiveness, anxiety, fall, injury, accident, bite, bleeding, inflammation, infection, drowsiness, insomnia, discomfort, stress, grooming, appearance, capability, performance, improvement, enhancement, curtailment, wellbeing, vitality, vigor, disability, phobia, malady, psychosis, environmental extremes, environmental exposure, dysfunction, disease symptom, chronic condition, mental acuity, emotional behavior, physical prowess, addiction, obsession, therapy, remedy, behavior, nutrition, diet, exercise, immunization, prevention, diagnosis, subscription, regimen, social performance, social interaction, endurance, sexual attribute, sexual performance, age-related attribute, age-related condition, prophylaxis, or treatment. In some embodiments, one or more accepting units 102 accept data 104 related to attentiveness, alertness, test performance, relaxation, pain, fever, attractiveness, anxiety, fall, injury, accident, bite, bleeding, inflammation, infection, drowsiness, insomnia, discomfort, stress, grooming, appearance, capability, performance, improvement, enhancement, curtailment, wellbeing, vitality, vigor, disability, phobia, malady, psychosis, environmental extremes, environmental exposure, dysfunction, disease symptom, chronic condition, mental acuity, emotional behavior, physical prowess, addiction, obsession, therapy, remedy, behavior, nutrition, diet, exercise, immunization, prevention, diagnosis, subscription, regimen, social performance, social interaction, endurance, sexual attribute, sexual performance, age-related attribute, age-related condition, prophylaxis, or treatment.

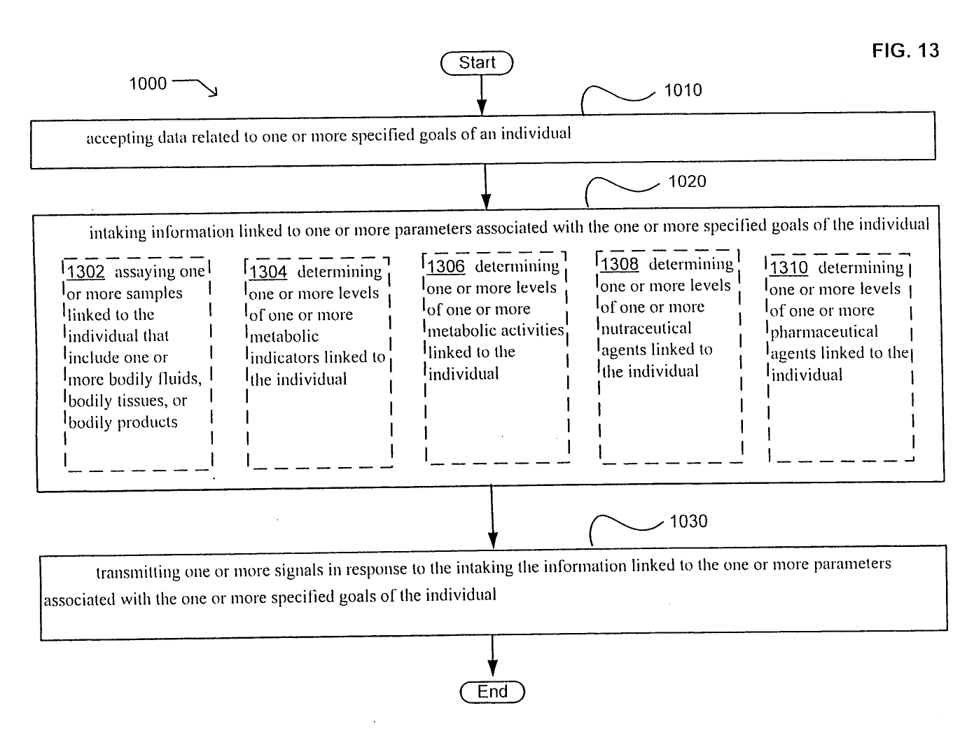

FIG. 13 illustrates alternative embodiments of the example operational flow 1000 of FIG. 10. FIG. 13 illustrates example embodiments where the intaking operation 1020 may include at least one additional operation. Additional operations may include an operation 1302, an operation 1304, an operation 1306, an operation 1308, and/or an operation 1310.

At operation 1304, the intaking operation 1020 may include assaying one or more samples linked to the individual that include one or more bodily fluids, bodily tissues, or bodily products. In some embodiments, one or more intaking units 110 can assay one or more samples linked to the individual 108 that include one or more bodily fluids, bodily tissues, or bodily products. Examples of such samples include, but are not limited to, blood, urine, saliva, synovial fluid, pleural fluid, peritoneal fluid, tears, mucus, ejaculate, skin, muscle, bone, hair, teeth, nails, urine, breath, fecal material, genital products, and the like.

At operation 1304, the intaking operation 1020 may include determining one or more levels of one or more metabolic indicators linked to the individual. In some embodiments, one or more intaking units 110 determine one or more levels of one or more metabolic indicators linked to the individual 108.

For example, vitamin B₁₂ and folate are two vitamins that have interdependent roles in nucleic acid synthesis. Deficiencies of either vitamin can cause megaloblastic anemia. Accordingly, in some embodiments, the levels of homocysteine and methylmalonic acid may be determined and used as metabolic indicators to indicate levels of vitamin B₁₂ and folate within an individual 108. In some embodiments, vitamin A deficiency may be assessed by determining albumin levels which are an indirect measure of vitamin A levels. In some embodiments, magnesium levels may be assessed directly. In some embodiments, magnesium levels may be assessed indirectly through analysis of insulin because magnesium deficiency results in impaired insulin secretion. Accordingly, in some embodiments, magnesium replacement may be used to restore insulin secretion. In some embodiments, fluorescent indicators may be used to determine chloride, zinc, and calcium levels as well as pH. Accordingly, numerous metabolic indicators that are linked to an individual 108 may be determined through use of known methods.

At operation 1306, the intaking operation 1020 may include determining one or more levels of one or more metabolic activities linked to the individual. In some embodiments, one or more intaking units 110 determine one or more levels of one or more metabolic activities linked to the individual 108.

In some embodiments, one or more intaking units 110 can be used to determine one or more enzyme activities of an individual 108. For example, when digested properly, protein supplies acidity to the blood. If an individual 108 is not able to adequately digest protein, the individual's 108 blood acquires excess alkaline reserves which must be continuously dumped via the kidneys into the urine. Accordingly, in some embodiments, blood alkalinity may be tested to assist in determining if an individual 108 is deficient in protease activity. In instances of protease deficiency, an individual 108 may wish to ingest a protease supplement to assist with digestion of protein. In some embodiments, enzyme activity may be determined directly. For example, protease activity can be measured directly through use of peptide substrates having an amino acid sequence that is recognized by a protease to be assayed. Such peptide substrates may be readily prepared or be obtained from commercial sources (i.e., Biotium, Inc., Hayward, Calif.; Biomol International Inc., Plymouth Meeting, Pa.; JPT Peptide Technologies, Inc., Springfield, Va.). In some embodiments, vitamin K deficiency exhibited by an individual 108 may be determined through measurement of Protein Induced by Vitamin K Absence (PIVK -A-II) using several known methods (Widdershoven J., Clin. Chem., 33(11):2074-2078 (1987)). Examples of such methods include, but are not limited to, electrophoresis-immunofixation and enzyme immunoassay. Numerous metabolic activities of an individual 108 may be determined through use of methods that are known and that have been described (i.e., Clinical Laboratory Medicine, Lippincott Williams and Wilkins, Philadelphia, Second Edition, 2002).

At operation 1308, the intaking operation 1020 may include determining one or more levels of one or more nutraceutical agents linked to the individual. In some embodiments, one or more intaking units 110 determine one or more levels of one or more nutraceutical agents 118 linked to the individual 108.

Numerous methods may be used to determine one or more nutraceutical agents 118 that are linked to an individual 108. Examples of such methods include, but are not limited to, chromatographic assay, mass spectrometry, spectrophotometry, immunological assay, and the like. These methods may be performed on numerous types of samples obtained from an individual 108 that are exemplified by bodily fluids, bodily tissues, bodily products and the like.

At operation 1310, the intaking operation 1020 may include determining one or more levels of one or more pharmaceutical agents linked to the individual. In some embodiments, one or more intaking units 110 determine one or more levels of one or more pharmaceutical agents linked to the individual 108.

Numerous methods may be used to determine one or more phannaceutical agents that are linked to an individual 108. Examples of such methods include, but are not limited to, chromatographic assay, mass spectrometry, spectrophotometry, immunological assay, and the like. These methods may be performed on numerous types of samples obtained from an individual 108 that are exemplified by bodily fluids, bodily tissues, bodily products and the like.

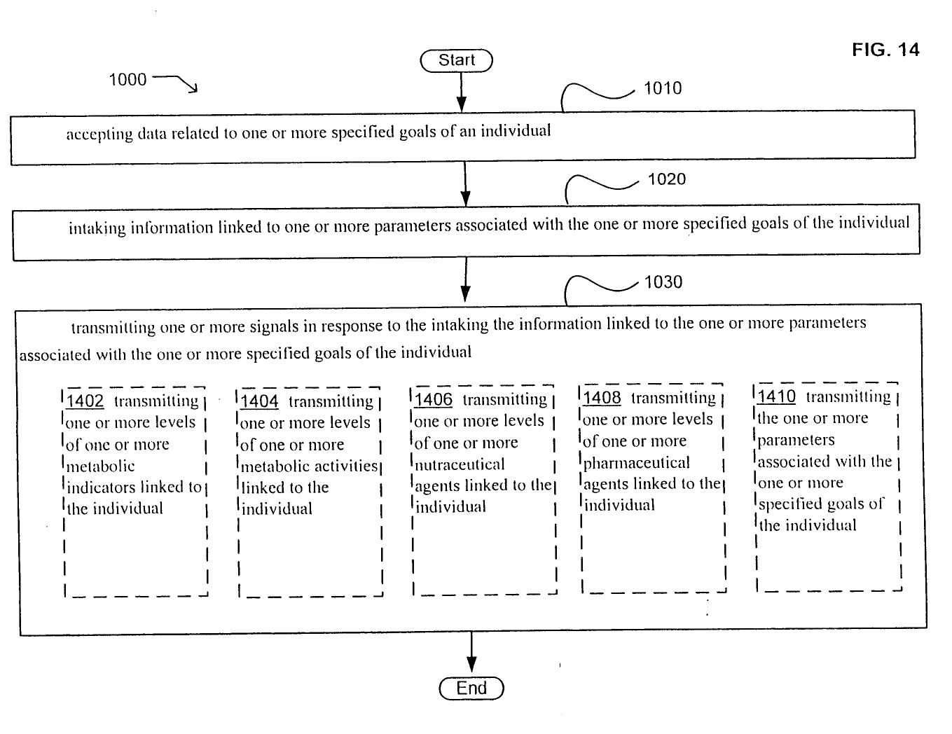

FIG. 14 illustrates alternative embodiments of the example operational flow 1000 of FIG. 10. FIG. 14 illustrates example embodiments where the transmitting operation 1030 may include at least one additional operation. Additional operations may include an operation 1402, an operation 1404, an operation 1406, an operation 1408, and/or an operation 1410.

At operation 1402, the transmitting operation 1030 may include transmitting one or more levels of one or more metabolic indicators linked to the individual. In some embodiments, one or more transmitting units 128 transmit one or more levels of one or more metabolic indicators linked to the individual 108.

In some embodiments, one or more transmitting units 128 may transmit one or more signals 130 indicating the levels of homocysteine and methylmalonic acid to indicate levels of vitamin B₁₂ and folate within an individual 108. In some embodiments, one or more transmitting units 128 may transmit one or more signals 130 indicating the level of albumin as an indirect measure of vitamin A levels. In some embodiments, one or more transmitting units 128 may transmit one or more signals 130 indicating the level of a metabolic indicator directly. For example, in some embodiments, one or more transmitting units 128 may transmit one or more signals 130 indicating vitamin A concentration, pH, magnesium concentration, calcium concentration, and the like. One or more transmitting units 128 may transmit one or more signals 130 indicating the level of numerous metabolic indicators.

At operation 1404, the transmitting operation 1030 may include transmitting one or more levels of one or more metabolic activities linked to the individual. In some embodiments, one or more transmitting units 128 transmit one or more levels of one or more metabolic activities linked to the individual 108.

In some embodiments, one or more transmitting units 128 can be used to transmit one or more signals 130 indicating the level of one or more enzyme activities of an individual 108. For example, in some embodiments, one or more transmitting units 128 can transmit one or more signals 130 indicating the level of a protease activity of an individual 108. In some embodiments, one or more transmitting units 128 can transmit one or more signals 130 indicating the level of Protein Induced by Vitamin K Absence (PIVKA-II) to indicate vitamin K deficiency or adequacy of an individual 108. One or more transmitting units 128 may transmit one or more signals 130 indicating the level of numerous metabolic activities linked to an individual 108.

At operation 1406, the transmitting operation 1030 may include transmitting one or more levels of one or more nutraceutical agents linked to the individual. In some embodiments, one or more transmitting units 128 transmit one or more levels of one or more nutraceutical agents 118 linked to the individual 108.

In some embodiments, one or more transmitting units 128 can transmit one or more signals 130 indicating one or more levels of one or more nutraceutical agents 118 that were determined at one or more times. For example, in some embodiments, one or more signals 130 indicating the level of a nutraceutical agent 118 at the time when administered to an individual 108, at time two hours post-administration, at time four hours post-administration, and at time six hours post-administration may be transmitted. Accordingly, in some embodiments, the rate at which one or more nutraceutical agents 118 are metabolized by an individual 108 may be determined. In some embodiments, the rate at which a level of nutraceutical is reached within an individual 108 may be determined. One or more signals 130 indicating numerous times and concentrations of nutraceutical agents 118 may be transmitted.

At operation 1408, the transmitting operation 1030 may include transmitting one or more levels of one or more pharmaceutical agents linked to the individual. In some embodiments, one or more transmitting units 128 transmit one or more levels of one or more pharmaceutical agents linked to the individual 108.

In some embodiments, one or more transmitting units 128 can transmit one or more signals 130 indicating one or more levels of one or more pharmaceutical agents that were determined at one or more times. For example, in some embodiments, one or more signals 130 indicating the level of a pharmaceutical agent at the time when administered to an individual 108, at time two hours post-administration, at time four hours post-administration, and at time six hours post-administration may be transmitted. Accordingly, in some embodiments, the rate at which one or more pharmaceutical agents are metabolized by an individual 108 may be determined. In some embodiments, the rate at which a level of a pharmaceutical agent is reached within an individual 108 may be determined. One or more signals 130 indicating numerous times and concentrations of pharmaceutical agents may be transmitted.

At operation 1410, the transmitting operation 1030 may include transmitting the one or more parameters associated with the one or more specified goals of the individual. In some embodiments, one or more transmitting units 128 transmit one or more parameters 114 associated with the one or more specified goals 106 of the individual 108.

One or more transmitting units 128 can transmit one or more signals 130 indicating numerous parameters 114 that are associated with one or more specified goals 106 of an individual 108. Examples of such parameters 114 include, but are not limited to, blood sugar levels, blood insulin levels, blood iron levels, hormone levels, nutraceutical agent 118 levels, pharmaceutical agent levels, cytokine levels, and the like (i.e., Fitzgerald et al., The Cytokine Facts Book, Academic Press, San Francisco, Second Edition, (2001)).

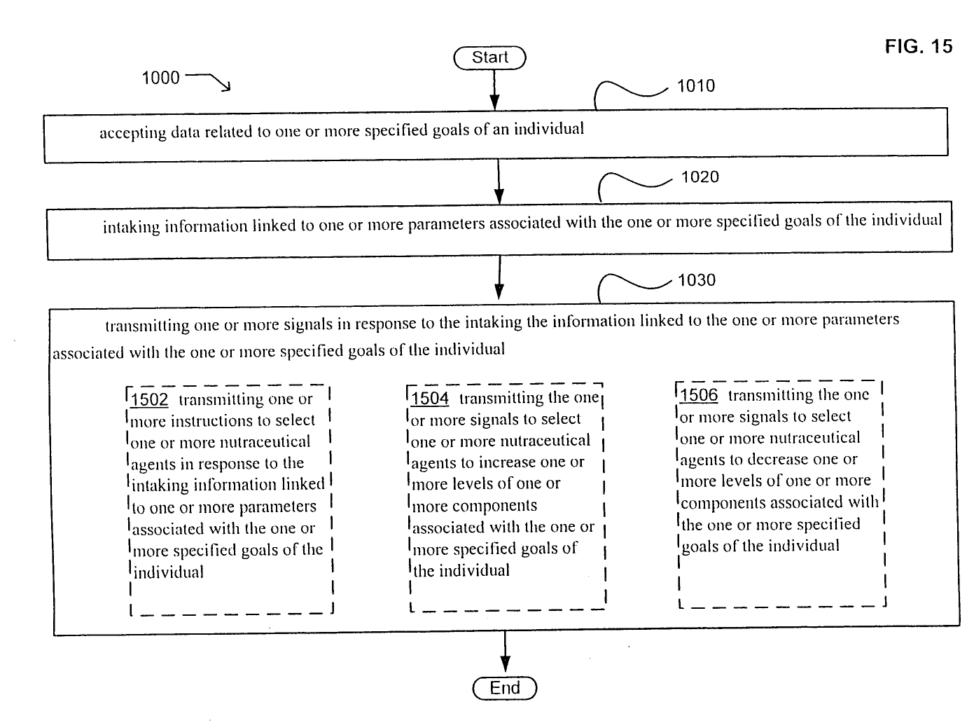

FIG. 15 illustrates alternative embodiments of the example operational flow 1000 of FIG. 10. FIG. 15 illustrates example embodiments where the transmitting operation 1030 may include at least one additional operation. Additional operations may include an operation 1502, an operation 1504, and/or an operation 1506.

At operation 1502, the transmitting operation 1030 may include transmitting one or more instructions to select one or more nutraceutical agents in response to the intaking information linked to one or more parameters associated with the one or more specified goals of the individual. In some embodiments, one or more transmitting units 128 transmit one or more instructions to select one or more nutraceutical agents 118 in response to the intaking information 112 linked to one or more parameters 114 associated with the one or more specified goals 106 of the individual 108.

In some embodiments, one or more transmitting units 128 transmit one or more signals 130 that include one or more instructions to select one or more nutraceutical agents 118 in response to accepting data 104 related to one or more specified goals 106 of an individual 108. One or more transmitting units 128 can transmit one or more signals 130 that include one or more instructions to select one or more nutraceutical agents 118 based on numerous parameters 114. Examples of such parameters 114 include, but are not limited to, blood sugar levels, blood insulin levels, blood iron levels, hormone levels, nutraceutical agent 118 levels, pharmaceutical agent levels, cytokine levels, and the like.

At operation 1504, the transmitting operation 1030 may include transmitting the one or more signals to select one or more nutraceutical agents to increase one or more levels of one or more components associated with the one or more specified goals of the individual. In some embodiments, one or more transmitting units 128 transmit one or more signals 130 to select one or more nutraceutical agents 118 to increase one or more levels of one or more components associated with the one or more specified goals 106 of the individual 108.

In some embodiments, one or more transmitting units 128 transmit one or more signals 130 to select one or more nutraceutical agents 118 that may directly complement one or more levels of one or more components associated with one or more specified goals 106 of the individual 108. For example, in some embodiments, one or more transmitting units 128 transmit one or more signals 130 to select calcium in various forms (i.e., calcium citrate, calcium aspartate, calcium carbonate) to avoid or reduce the effects of osteoporosis. In another embodiment, one or more transmitting units 128 transmit one or more signals 130 to select chromium to lower blood sugar, increase insulin sensitivity, reduce body fat, control hunger, suppress appetite, increase lean body/muscle mass, or substantially any combination thereof. In some embodiments, one or more transmitting units 128 transmit one or more signals 130 to select one or more nutraceutical agents 118 to indirectly supplement a component associated with one or more specified goals 106 of an individual 108. For example, one or more transmitting units 128 may transmit one or more signals 130 to select 5-hydroxy-tryptophan for administration to an individual 108 if the individual 108 suffers from depression and is found to exhibit low levels of serotonin. Numerous signals 130 may be transmitted to select one or more nutraceutical agents 118 to increase one or more levels of one or more components associated with one or more specified goals 106 of an individual 108.

At operation 1506, the transmitting operation 1030 may include transmitting the one or more signals to select one or more nutraceutical agents to decrease one or more levels of one or more components associated with the one or more specified goals of the individual. In some embodiments, one or more transmitting units 128 transmit one or more signals 130 to select one or more nutraceutical agents 118 to decrease one or more levels of one or more components associated with the one or more specified goals 106 of the individual 108.

Low density lipoproteins are examples of components that are linked to vascular disease. Accordingly, in some embodiments, one or more transmitting units 128 may transmit one or more signals 130 to select one or more nutraceutical agents 18 that will act to lower the low density lipoprotein concentration of an individual 108. Examples of such nutraceutical agents 118 include, but are not limited to, plant stanols, dietary fiber, stanol esters, isoflavones, diallyl sulfides, niacin, soy protein, and substantially any combination thereof. Glucose is an example of a component that is linked to hyperglycemia. Accordingly, in some embodiments, one or more transmitting units 128 may transmit one or more signals 130 to select buckwheat to lower blood glucose levels of an individual 108. Free radical oxygen species that include singlet oxygen, hydroxyl radicals, peroxides, and superoxide radicals are components that act to damage lipids and other cellular structures. Accordingly, in some embodiments, one or more transmitting units 128 may transmit one or more signals 130 to select one or more nutraceutical agents 118 to reduce levels of free radicals within an individual 108. Examples of such nutraceutical agents 118 include, but are not limited to, terpenes, carotenoids, limonoids, phenols, flavonoids, isoprenoids, and the like. One or more transmitting units 128 can transmit one or more signals 130 to select numerous types of nutraceutical agents 118 to decrease one or more levels of one or more components associated with the one or more specified goals 106 of the individual 108.

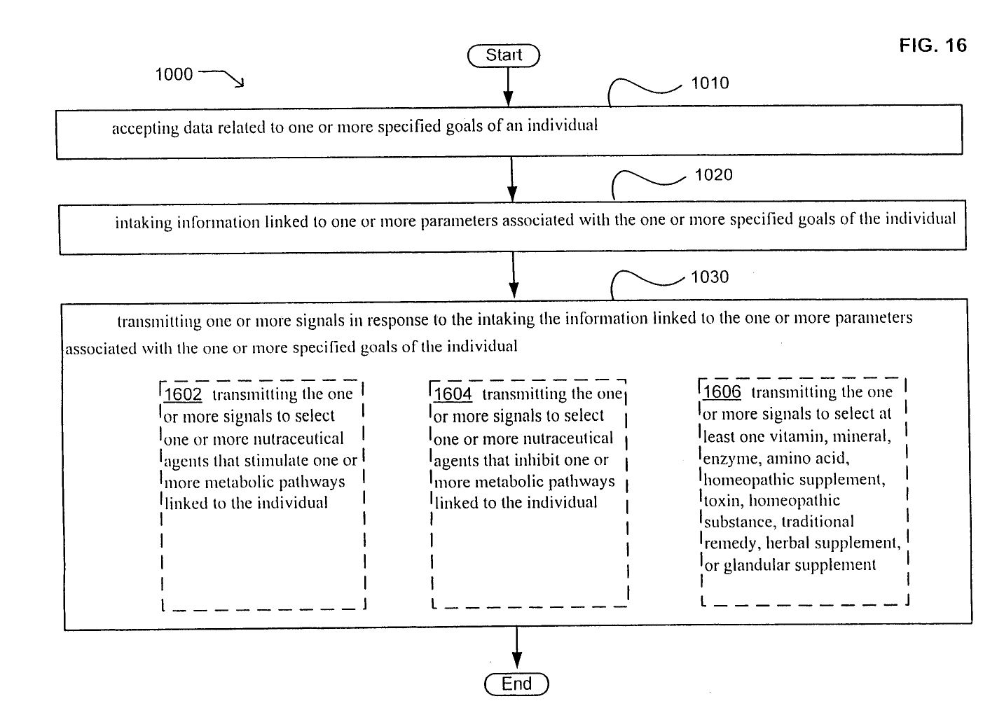

FIG. 16 illustrates alternative embodiments of the example operational flow 1000 of FIG. 10. FIG. 16 illustrates example embodiments where the transmitting operation 1030 may include at least one additional operation. Additional operations may include an operation 1602, an operation 1604, and/or an operation 1606.

At operation 1602, the transmitting operation 1030 may include transmitting the one or more signals to select one or more nutraceutical agents that stimulate one or more metabolic pathways linked to the individual. In some embodiments, one or more transmitting units 128 transmit one or more signals 130 to select one or more nutraceutical agents 118 that stimulate one or more metabolic pathways linked to the individual 108.

In some embodiments, one or more transmitting units 128 can transmit one or more signals 130 to select one or more nutraceutical agents 118 that stimulate one or more metabolic pathways associated with one or more specified goals 106 of an individual 108. For example, in some embodiments, decline in L-carnitine synthesis is thought to be linked to senile reduction in bone synthesis (Colucci et al., Calcified Tissue International, 76:458-465 (2005)). Dihydroepiandrosterone (DHEA) is thought to affect levels of L-carnitine through promoting the expression of carnitine-synthesizing enzymes (Chiu et al., Calcified Tissue International, 64:527-533 (1999)). Accordingly, in some embodiments, one or more transmitting units 128 can transmit one or more signals 130 to select DHEA to stimulate the L-carnitine synthetic pathway to reduce senile reduction in bone synthesis. In other embodiments, one or more transmitting units 128 can transmit one or more signals 130 to select buckwheat extract to lower blood glucose through activation of mitogen activated protein kinase via phospholipase D (Appleton and Lockwood, The Pharmaceutical Journal, 277:78-83 (2006)). In another embodiment, resveratrol has been shown to stimulate endothelial nitric oxide synthase activity (Klinge et al., J. Biol. Chem., 280(9):7460-7468 (2005); Wallerath et al., Circulation, 106(13):1652-1658 (2002)). Endothelial nitric oxide synthase is an enzyme that catalyzes the formation of nitric oxide by vascular endothelial cells. Nitric oxide is needed to maintain arterial relaxation (vasodilation), and impaired nitric oxide-dependent vasodilation is associated with increased risk of cardiovascular disease (Duffy and Vita, Curr. Opin. Lipidol., 14(1):21-27 (2003)). Accordingly, in some embodiments, one or more transmitting units 128 transmit one or more signals 130 to select resveratrol to reduce cardiovascular disease. One or more transmitting units 128 can transmit one or more signals 130 to select numerous types of nutraceutical agents 118 to stimulate one or more metabolic pathways linked to the individual 108.

At operation 1604, the transmitting operation 1030 may include transmitting the one or more signals to select one or more nutraceutical agents that inhibit one or more metabolic pathways linked to the individual. In some embodiments, one or more transmitting units 128 transmit one or more signals 130 to select one or more nutraceutical agents 118 that inhibit one or more metabolic pathways linked to the individual 108.

In some embodiments, one or more transmitting units 128 can transmit one or more signals 130 to select one or more nutraceutical agents 118 that inhibit one or more enzymes that participate within a metabolic pathway. Such metabolic pathways may be involved with a specified goal 106 of an individual 108. For example, angiotensin-converting enzyme has been linked to high blood pressure. Flavonoids have been shown to inhibit angiotensin-converting enzyme (Actis-Goretta et al., J. Agric. Food Chem., 54(1):229-234 (2006)). Accordingly, in some embodiments, one or more transmitting units 128 can transmit one or more signals 130 to select flavonoids to assist in the reduction of blood pressure (Li et al., Chin. J. Physiol., 48(2):101-106 (2005); Machha and Mustafa, J. Cardiovasc. Pharmacol., 46(1):36-40 (2005)). Genistein, one of the predominant soy isoflavones, has been shown to compete with 17beta-estradiol for estrogen receptor binding because of its structural similarity, resulting in agonistic or antagonistic activity. This has been shown to cause inhibition of cell growth in breast and prostate cancers in vivo and in vitro. Accordingly, in some embodiments, one or more transmitting units 128 can transmit one or more signals 130 to select soy isoflavones containing genistein as a nutraceutical agent 118 for cancer chemoprevention (Sarkar et al., Mini Rev. Med. Chem., 6(4):401-407 (2006)). The isoflavonoids, daidzin, daidzein, and puerarin have been shown to reduce alcohol consumption (Lin R C, Alcohol Clin. Exp. Res., 20(4):659-663 (1996)). A link between daidzin's capacity to reduce alcohol consumption and its ability to increase the liver mitochondrial monoamine oxidase: aldehyde dehydrogenase activity ratio has been established (Keung, Med. Res. Rev., 23(6):669-696 (2003)). This increase in ratio is thought to occur through inhibition of aldehyde dehydrogenase activity. Accordingly, in some embodiments, one or more transmitting units 128 can transmit one or more signals 130 to select isoflavonoid mixtures that include daidzin, daidzein, and/or puerarin to lower alcohol consumption. One or more transmitting units 128 can transmit one or more signals 130 to select numerous other types of nutraceutical agents 118 that inhibit one or more metabolic pathways linked to the individual 108.

At operation 1606, the transmitting operation 1030 may include transmitting the one or more signals to select at least one vitamin, mineral, enzyme, amino acid, homeopathic supplement, toxin, homeopathic substance, traditional remedy, herbal supplement, or glandular supplement. In some embodiments, one or more transmitting units 128 transmit one or more signals 130 to select at least one vitamin, mineral, enzyme, amino acid, homeopathic supplement, toxin, homeopathic substance, traditional remedy, herbal supplement, or glandular supplement.

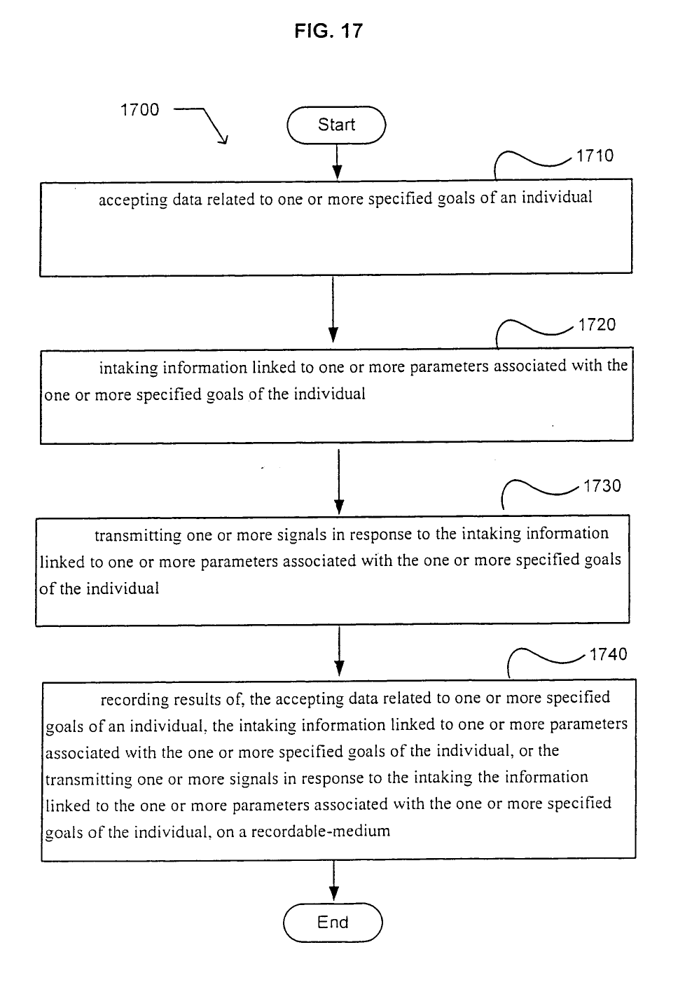

FIG. 17 illustrates an operational flow 1700 that includes an accepting operation 1710, an intaking operation 1720, and a transmitting operation 1730 (which correspond to the accepting operation 1010, the intaking operation 1020, and the transmitting operation 1030 illustrated in FIG. 10) with an additional recording operation 1740. In FIG. 17, discussion and explanation may be provided with respect to the above-described example of FIG. 1, and/or with respect to other examples and contexts. However, it should be understood that the operations may be executed in a number of other environments and contexts, and/or modified versions of FIG. 1. Also, although the various operations are presented in the sequence(s) illustrated, it should be understood that the various operations may be performed in other orders than those which are illustrated, or may be performed concurrently.

After a start operation, the operational flow 1700 includes the operations 1710, 1720, and 1730 (which correspond to operations 1010, 1020, and 1030 as described with regard to FIG. 10) and an additional recording operation 1740 involving recording results of, the accepting data related to one or more specified goals of an individual, the intakin q information linked to one or more parameters associated with the one or more specified goals of the individual, or the transmitting one or more signals in response to the intaking the information linked to the one or more parameters associated with the one or more specified goals of the individual, on a recordable-medium. In some embodiments, one or more recording units 124 may record results of, the accepting data 104 related to one or more specified goals 106 of an individual 108, the intakin r information 112 linked to one or more parameters 114 associated with the one or more specified goals 106 of the individual 108, or the transmitting one or more signals 130 in response to the intaking information 112 linked to one or more parameters 114 associated with the one or more specified goals 106 of the individual 108, on a recordable-medium 126.

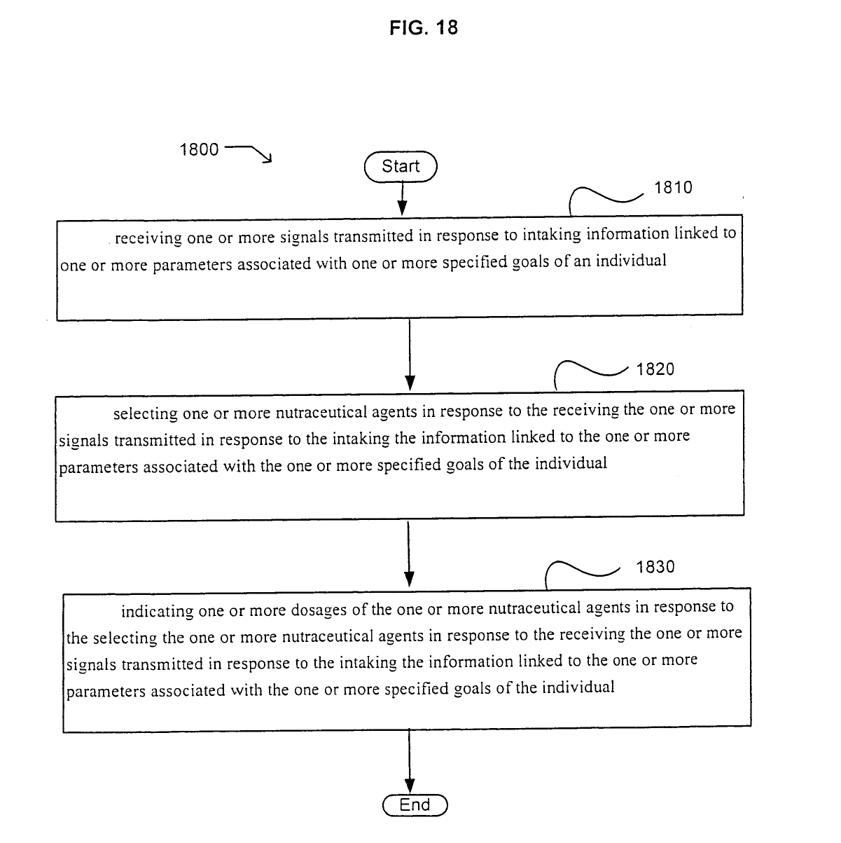

FIG. 18 illustrates an operational flow 1800 representing examples of operations that are related to the performance of a method for nutraceutical agent 118 and dosing. In FIG. 18 and in following figures that include various examples of operations used during performance of the method, discussion and explanation may be provided with respect to the above-described example of FIG. 1, and/or with respect to other examples and contexts. However, it should be understood that the operations may be executed in a number of other environments and contexts, and/or modified versions of FIG. 1. Also, although the various operations are presented in the sequence(s) illustrated, it should be understood that the various operations may be performed in other orders than those which are illustrated, or may be performed concurrently.

After a start operation, the operational flow 1800 includes a receiving operation 1810 involving receiving one or more signals transmitted in response to intaking information linked to one or more parameters associated with one or more specified goals of an individual. In some embodiments, one or more receiving units 132 receive one or more signals 130 transmitted in response to intaking information 112 linked to one or more parameters 114 associated with one or more specified goals 106 of the individual 108.

In some embodiments, one or more receiving units 132 may receive one or more signals 130 that include data 104 related to one or more specified goals 106 of an individual 108. In some embodiments, one or more receiving units 132 receive one or more signals 130 indicating one or more levels of one or more metabolic indicators linked to the individual 108. In some embodiments, one or more receiving units 132 receive one or more signals 130 indicating one or more metabolic activities linked to the individual 108. In some embodiments, one or more receiving units 132 receive one or more signals 130 indicating one or more levels of one or more nutraceutical agents 118 linked to the individual 108. In some embodiments, one or more receiving units 132 receive one or more signals 130 indicating one or more levels of one or more pharmaceutical agents linked to the individual 108. In some embodiments, one or more receiving units 132 receive one or more parameters 114 associated with the one or more specified goals 106 of the individual 108. In some embodiments, one or more receiving units 132 receive one or more instructions to select one or more nutraccutical agents 118 in response to the intaking information 112 linked to one or more parameters 114 associated with the one or more specified goals 106 of the individual 108. In some embodiments, one or more receiving units 132 receive one or more signals 130 to select one or more nutraceutical agents 118 to increase one or more levels of one or more components associated with the one or more specified goals 106 of the individual 108. In some embodiments, one or more receiving units 132 receive one or more signals 130 to select one or more nutraceutical agents 118 to decrease one or more levels of one or more components associated with the one or more specified goals 106 of the individual 108. In some embodiments, one or more receiving units 132 receive one or more signals 130 to select one or more nutraceutical agents 118 that stimulate one or more metabolic pathways linked to the individual 108. In some embodiments, one or more receiving units 132 receive one or more signals 130 to select one or more nutraceutical agents 118 that inhibit one or more metabolic pathways linked to the individual 108. In some embodiments, one or more receiving units 132 receive one or more signals 130 to select at least one vitamin, mineral, enzyme, amino acid, homeopathic supplement, toxin, homeopathic substance, traditional remedy, herbal supplement, glandular supplement, or substantially any combination thereof.

In some embodiments, one or more receiving units 132 may receive one or more signals 130 that include data 104 related to one or more specified goals 106 of an individual 108. One or more receiving units 132 may receive one or more signals 130 through use of numerous technologies. Examples of such technologies include, but are not limited to, wireless transmission, telephone, internet transmission, digital transmission, analog transmission, optical transmission, and the like.

After a start operation, the operational flow 1800 includes a selecting operation 1820 involving selecting one or more nutraceutical agents in response to the receiving the one or more signals transmitted in response to the intaking the information linked to the one or more parameters associated with the one or more specified goals of the individual. In some embodiments, one or more selecting units 116 select one or more nutraceutical agents 118 in response to the receiving one or more signals 130 transmitted in response to intaking information 112 linked to one or more parameters 114 associated with one or more specified goals 106 of the individual 108.

In some embodiments, one or more selecting units 116 may select one or more nutraceutical agents 118 in response to the intaking information 112 linked to one or more parameters 114 associated with the one or more specified goats 106 of the individual 108. In some embodiments, one or more selecting units 116 may select one or more nutraceutical agents 118 in response to one or more intaking units 110. In some embodiments, one or more selecting units 116 may select one or more nutraceutical agents 118 in response to one or more accepting units 102. In some embodiment one or more selecting units 116 may select one or more nutraceutical agents 118 in response to one or more intaking units 110 and one or more accepting units 102. Accordingly, in some embodiments, one or more selecting units 116 may select one or more nutraceutical agents 118 with regard to one or more characteristics of the individual 108 and one or more parameters 114 associated with one or more specified goals 106 of the individual 108. For example, in some embodiments, one or more selecting units 116 may select one or more nutraceutical agents 118 based on the age of the individual 108 and the level of testosterone in the individual's 108 blood. In other embodiments, one or more selecting units 116 may select one or more nutraceutical agents 118 based on insurance coverage held by an individual 108 and a specified goal 106 of the individual 108. Accordingly, numerous combinations of information 112 and data 104 may be used by one or more selecting units 116 to select one or more nutraceutical agents 118.

After a start operation, the operational flow 1800 includes an indicating operation 1830 involving indicating one or more dosages of the one or more nutraceutical agents in response to the selecting the one or more nutraceutical agents in response to the receiving the one or more signals transmitted in response to the intaking the information linked to the one or more parameters associated with the one or more specified goals of the individual. In some embodiments, one or more indicating units 120 indicate one or more dosages 122 of the one or more nutraceutical agents 118 in response to the selecting one or more nutraceutical agents 118 in response to the receiving one or more signals 130 transmitted in response to intaking information 112 linked to one or more parameters 114 associated with one or more specified goals 106 of the individual 108.

In some embodiments, one or more indicating units 120 may indicate one or more dosages 122 of the one or more nutraceutical agents 118 in response to the intaking information 112 linked to one or more parameters 114 associated with the one or more specified goals 106 of the individual 108. In some embodiments, one or more indicating units 120 may indicate one or more dosages 122 of one or more nutraceutical agents 118 in human-readable format. In some embodiments, one or more indicating units 120 may indicate one or more dosages 122 of one or more nutraceutical agents 118 in machine-readable format. In some embodiments, one or more indicating units 120 may deposit one or more dosages 122 of one or more nutraceutical agents 118 on one or more labels. In some embodiments, one or more indicating units 120 may dispense one or more nutraceutical agents 118. In some embodiments, one or more indicating units 120 may dispense one or more nutraceutical agents 118 in unit dosage form. In some embodiments, one or more indicating units 120 may dispense two or more of one or more nutraceutical agents 118 in a single administration form.

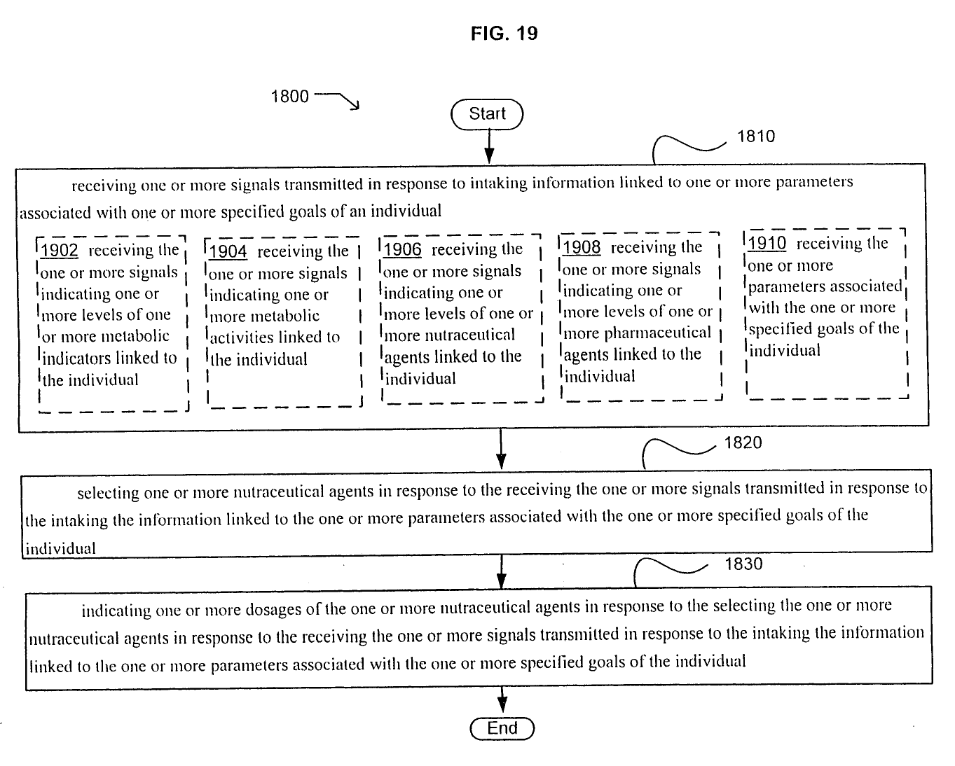

FIG. 19 illustrates alternative embodiments of the example operational flow 1800 of FIG. 18. FIG. 19 illustrates example embodiments where the receiving operation 1810 may include at least one additional operation. Additional operations may include an operation 1902, an operation 1904, an operation 1906, an operation 1908, and/or an operation 1910.

At operation 1902, the receiving operation 1810 may include receiving the one or more signals indicating one or more levels of one or more metabolic indicators linked to the individual. In some embodiments, one or more receiving units 132 receive one or more signals 130 indicating one or more levels of one or more metabolic indicators linked to the individual 108.

In some embodiments, one or more receiving units 132 may receive one or more signals 130 indicating the levels of homocysteine and methylmalonic acid to indicate levels of vitamin B₁₂, and folate within an individual 108. In some embodiments, one or more receiving units 132 may receive one or more signals 130 indicating the level of albumin as an indirect measure of vitamin A levels. In some embodiments, one or more receiving units 132 may receive one or more signals 130 indicating the level of a metabolic indicator directly. For example, in some embodiments, one or more receiving units 132 mav receive one or more signals 130 indicating vitamin A concentration, pH, magnesium concentration, calcium concentration, and the like. One or more receiving units 132 may receive one or more signals 130 indicating the level of numerous metabolic indicators.

At operation 1904, the receiving operation 1810 may include receiving the one or more signals indicating one or more metabolic activities linked to the individual. In some embodiments, one or more receiving units 132 receive one or more signals 130 indicating one or more metabolic activities linked to the individual 108.

In some embodiments, one or more receiving units 132 can be used to receive one or more signals 130 that indicate the level of one or more enzyme activities of an individual 108. For example, in some embodiments, one or more receiving units 132 can receive one or more signals 130 that indicate the level of a protease activity of an individual 108. In some embodiments, one or more receiving units 132 can receive one or more signals 130 that indicate the level of Protein Induced by Vitamin K Absence (PIVKA-II) to indicate vitamin K deficiency or adequacy of an individual 108. One or more receiving units 132 may receive one or more signals 130 that indicate the level of numerous metabolic activities linked to an individual 108.

At operation 1906, the receiving operation 1810 may include receiving the one or more signals indicating one or more levels of one or more nutraceutical agents linked to the individual. In some embodiments, one or more receiving units 132 receive one or more signals 130 indicating one or more levels of one or more nutraceutical agents 118 linked to the individual 108.

In some embodiments, one or more receiving units 132 can receive one or more signals 130 indicating one or more levels of one or more nutraceutical agents 118 that were determined at one or more times. For example, in some embodiments, one or more signals 130 indicating the level of a nutraceutical agent 118 at the time when administered to an individual 108, at time two hours post-administration, at time four hours post-administration, and at time six hours post-administration may be received. Accordingly, in some embodiments, the rate at which one or more nutraceutical agents 118 are metabolized by an individual 108 may be determined. In some embodiments, the rate at which a level of nutraceutical is reached within an individual 108 may be determined. One or more signals 130 indicating numerous times and concentrations of nutraceutical agents 118 may be received.

At operation 1908, the receiving operation 1810 may include receiving the one or more signals indicating one or more levels of one or more pharmaceutical agents linked to the individual. In some embodiments, one or more receiving units 132 receive one or more signals 130 indicating one or more levels of one or more pharmaceutical agents linked to the individual 108.

In some embodiments, one or more receiving units 132 can receive one or more signals 130 indicating one or more levels of one or more pharmaceutical agents that were determined at one or more times. For example, in some embodiments, one or more signals 130 indicating the level of a pharmaceutical agent at the time when administered to an individual 108, at time two hours post-administration, at time four hours post-administration, and at time six hours post-administration may be received. Accordingly, in some embodiments, the rate at which one or more pharmaceutical agents are metabolized by an individual 108 may be determined. In some embodiments, the rate at which a level of a pharmaceutical agent is reached within an individual 108 may be determined. One or more signals 130 indicating numerous times and concentrations of pharmaceutical agents may be received.

At operation 1910, the receiving operation 1810 may include receiving the one or more parameters associated with the one or more specified goals of the individual. In some embodiments, one or more receiving units 132 receive one or more parameters 114 associated with the one or more specified goals 106 of the individual 108.

One or more receiving units 132 can receive one or more signals 130 indicating numerous parameters 114 that are associated with one or more specified goals 106 of an individual 108. Examples of such parameters 114 include, but are not limited to, blood sugar levels, blood insulin levels, blood iron levels, hormone levels, nutraceutical agent 118 levels, pharmaceutical agent levels, cytokine levels, and the like (i.e., Fitzgerald et al., The Cytokine Facts Book, Academic Press, San Francisco, Second Edition, (2001)).

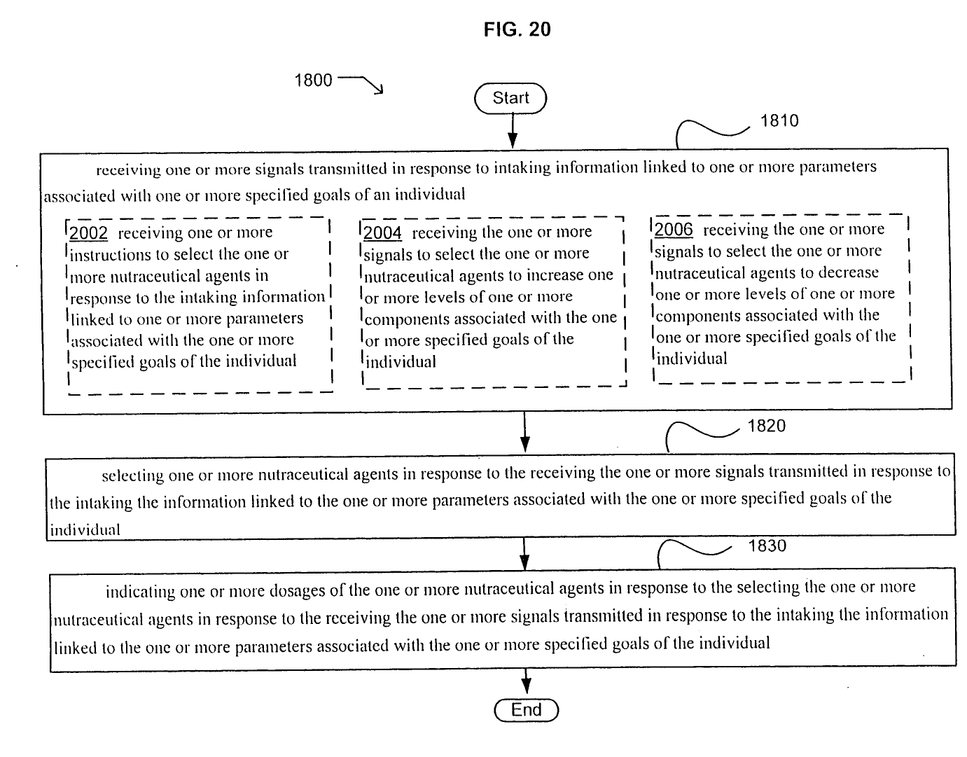

FIG. 20 illustrates alternative embodiments of the example operational flow 1800 of FIG. 18. FIG. 20 illustrates example embodiments where the receiving operation 1810 may include at least one additional operation. Additional operations may include an operation 2002, an operation 2004, and/or an operation 2006.

At operation 2002, the receiving operation 1810 may include receiving one or more instructions to select the one or more nutraceutical agents in response to the intaking information linked to one or more parameters associated with the one or more specified goals of the individual. In some embodiments, one or more receiving units 132 receive one or more instructions to select one or more nutraceutical agents 118 in response to the intaking information 112 linked to one or more parameters 114 associated with the one or more specified goals 106 of the individual 108.

In some embodiments, one or more receiving units 132 receive one or more signals 130 that include one or more instructions to select one or more nutraceutical agents 118 in response to accepting data 104 related to one or more specified goals 106 of an individual 108. One or more receiving units 132 can receive one or more signals 130 that include one or more instructions to select one or more nutraceutical agents 18 based on numerous parameters 114. Examples of such parameters 114 include, but are not limited to, blood sugar levels, blood insulin levels, blood iron levels, hormone levels, nutraceutical agent 18 levels, pharmaceutical agent levels, cytokine levels, and the like.

At operation 2004, the receiving operation 1810 may include receiving the one or more signals to select the one or more nutraceutical agents to increase one or more levels of one or more components associated with the one or more specified goals of the individual. In some embodiments, one or more receiving units 132 receive one or more signals 130 to select one or more nutraceutical agents 118 to increase one or more levels of one or more components associated with the one or more specified goals 106 of the individual 108.

In some embodiments, one or more receiving units 132 receive one or more signals 130 to select one or more nutraceutical agents 118 that may directly complement one or more levels of one or more components associated with one or more specified goals 106 of the individual 108. For example, in some embodiments, one or more receiving units 132 receive one or more signals 130 to select calcium in various forms (i.e., calcium citrate, calcium aspartate, calcium carbonate) to avoid or reduce the effects of osteoporosis. In another embodiment, one or more receiving units 132 receive one or more signals 130 to select chromium to lower blood sugar, increase insulin sensitivity, reduce body fat, control hunger, suppress appetite, increase lean body/muscle mass, or substantially any combination thereof. In some embodiments, one or more receiving units 132 receive one or more signals 130 to select one or more nutraceutical agents 118 to indirectly supplement a component associated with one or more specified goals 106 of an individual 108. For example, one or more receiving units 132 may receive one or more signals 130 to select 5-hydroxy-tryptophan for administration to an individual 108 if the individual 108 suffers from depression and is found to exhibit low levels of serotonin. Numerous signals 130 may be received to select one or more nutraceutical agents 118 to increase one or more levels of one or more components associated with one or more specified goals 106 of an individual 108.

At operation 2006, the receiving operation 1810 may include receiving the one or more signals to select the one or more nutraceutical agents to decrease one or more levels of one or more components associated with the one or more specified goals of the individual. In some embodiments, one or more receiving units 132 receive one or more signals 130 to select one or more nutraceutical agents 118 to decrease one or more levels of one or more components associated with the one or more specified goals 106 of the individual 108.

Low density lipoproteins are examples of components that are linked to vascular disease. Accordingly, in some embodiments, one or more receiving units 132 may receive one or more signals 130 to select one or more nutraceutical agents 118 that will act to lower the low density lipoprotein concentration of an individual 108. Examples of such nutraceutical agents 118 include, but are not limited to, plant stanols, dietary fiber, stanol esters, isoflavones, diallyl sulfides, niacin, soy protein, and substantially any combination thereof. Glucose is an example of a component that is linked to hyperglycemia. Accordingly, in some embodiments, one or more receiving units 132 may receive one or more signals 130 to select buckwheat to lower blood glucose levels of an individual 108. Free radical oxygen species that include singlet oxygen, hydroxyl radicals, peroxides, and superoxide radicals are components that act to damage lipids and other cellular structures. Accordingly, in some embodiments, one or more receiving units 112 may receive one or more signals 130 to select one or more nutraceutical agents 118 to reduce levels of free radicals within an individual 108. Examples of such nutraceutical agents 118 include, but are not limited to, terpenes, carotenoids, limonoids, phenols, flavonoids, isoprenoids, and the like. One or more receiving units 132 can receive one or more signals 130 to select numerous types of nutraceutical agents 118 to decrease one or more levels of one or more components associated with the one or more specified goals 106 of the individual 108.

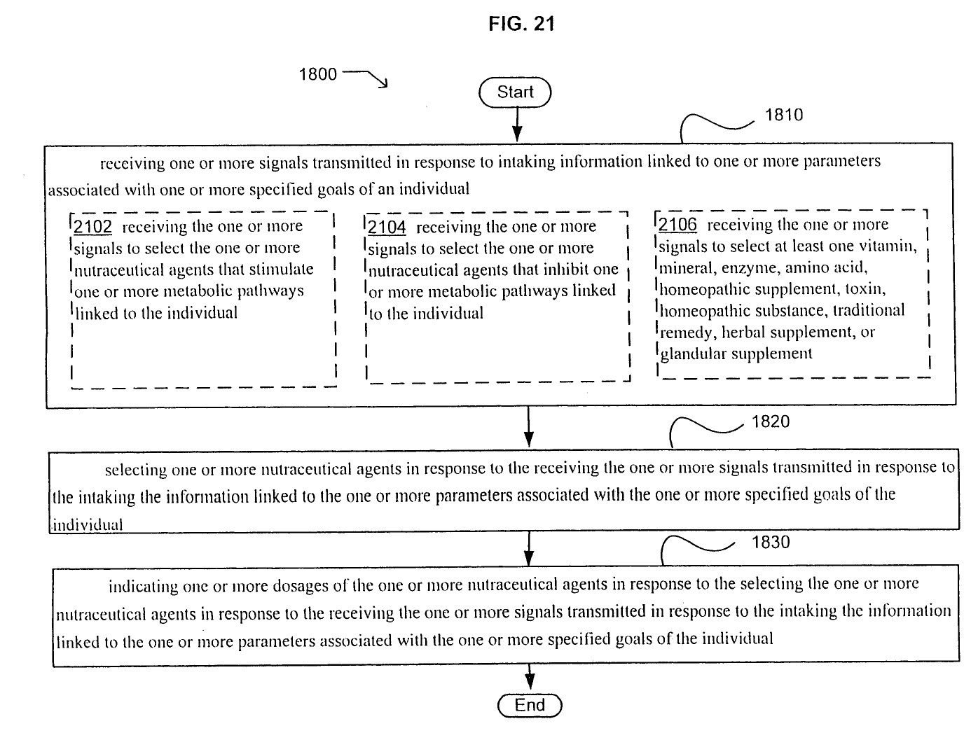

FIG. 21 illustrates alternative embodiments of the example operational flow 1800 of FIG. 18. FIG. 21 illustrates example embodiments where the receiving operation 1810 may include at least one additional operation. Additional operations may include an operation 2102, an operation 2104, and/or an operation 2106.

At operation 2102, the receiving operation 1810 may include receiving the one or more signals to select the one or more nutraceutical agents that stimulate one or more metabolic pathways linked to the individual. In some embodiments, one or more receiving units 132 receive one or more signals 130 to select one or more nutraceutical agents 118 that stimulate one or more metabolic pathways linked to the individual 108.

In some embodiments, one or more receiving units 132 can receive one or more signals 130 to select one or more nutraceutical agents 118 that stimulate one or more metabolic pathways associated with one or more specified goals 106 of an individual 108. For example, in some embodiments, decline in L-carnitine synthesis is thought to be linked to senile reduction in bone synthesis (Colucci et al., Calcified Tissue International, 76:458-465 (2005)). Dihydroepiandrosterone (DHEA) is thought to affect levels of L-carnitine through promoting the expression of carnitine-synthesizing enzymes (Chiu et al., Calcified Tissue International, 64:527-533 (1999)). Accordingly, in some embodiments, one or more receiving units 132 can receive one or more signals 130 to select DHEA to stimulate the L-carnitine synthetic pathway to reduce senile reduction in bone synthesis. In other embodiments, one or more receiving units 132 can receive one or more signals 130 to select buckwheat extract to lower blood glucose through activation of mitogen activated protein kinase via phospholipase D (Appleton and Lockwood, The Pharmaceutical Journal, 277:78-83 (2006)). In another embodiment, resveratrol has been shown to stimulate endothelial nitric oxide synthase activity (Klinge et al., J. Biol. Chem., 280(9):7460-7468 (2005); Wallerath et al., Circulation, 106(13):1652-1658 (2002)). Endothelial nitric oxide synthase is an enzyme that catalyzes the formation of nitric oxide by vascular endothelial cells. Nitric oxide is needed to maintain arterial relaxation (vasodilation), and impaired nitric oxide-dependent vasodilation is associated with increased risk of cardiovascular disease (Duffy and Vita, Curr. Opin. Lipidol., 14(1):21-27 (2003)). Accordingly, in some embodiments, one or more receiving units 132 receive one or more signals 130 to select resveratrol to reduce cardiovascular disease. One or more receiving units 132 can receive one or more signals 130 to select numerous types of nutraceutical agents 118 to stimulate one or more metabolic pathways linked to the individual 108.

At operation 2104, the receiving operation 1810 may include receiving the one or more signals to select the one or more nutraceutical agents that inhibit one or more metabolic pathways linked to the individual. In some embodiments, one or more receiving units 132 receive one or more signals 130 to select one or more nutraceutical agents 118 that inhibit one or more metabolic pathways linked to the individual 108.

In some embodiments, one or more receiving units 132 can receive one or more signals 130 to select one or more nutraceutical agents 118 that inhibit one or more enzymes that participate within a metabolic pathway. Such metabolic pathways may be involved with a specified goal 106 of an individual 108. For example, angiotensin-converting enzyme has been linked to high blood pressure. Flavonoids have been shown to inhibit angiotensin-converting enzyme (Actis-Goretta et al., J. A gric. Food Chem., 54(1):229-234 (2006)). Accordingly, in some embodiments, one or more receiving units 132 can receive one or more signals 130 to select flavonoids to assist in the reduction of blood pressure (Li et al., Chin. J. Physiol., 48(2):101-106 (2005); Machha and Mustafa, J. Cardiovasc. Pharmacol., 46(1):36-40 (2005)). Genistein, one of the predominant soy isoflavones, has been shown to compete with 17beta-estradiol for estrogen receptor binding because of its structural similarity, resulting in agonistic or antagonistic activity. This has been shown to cause inhibition of cell growth in breast and prostate cancers in vivo and in vitro. Accordingly, in some embodiments, one or more receiving units 132 can receive one or more signals 130 to select soy isoflavones containing genistein as a nutraceutical agent 118 for cancer chemoprevention (Sarkar et al., Mini Rev. Med. Chem., 6(4):401-407 (2006)). The isoflavonoids, daidzin, daidzein, and puerarin have been shown to reduce alcohol consumption (Lin R C, Alcohol Clin. Exp. Res., 20(4):659-663 (1996)). A link between daidzin's capacity to reduce alcohol consumption and its ability to increase the liver mitochondrial monoamine oxidase: aldehyde dehydrogenase activity ratio has been established (Keung, Med. Res. Rev., 23(6):669-696 (2003)). This increase in ratio is thought to occur through inhibition of aldehyde dehydrogenase activity. Accordingly, in some embodiments, one or more receiving units 132-can receive one or more signals 130 to select isoflavonoid mixtures that include daidzin, daidzein, and/or puerarin to lower alcohol consumption. One or more receiving units 132 can receive one or more signals 130 to select numerous other types of nutraceutical agents 118 that inhibit one or more metabolic pathways linked to the individual 108.

At operation 2106, the receiving operation 1810 may include receiving the one or more signals to select at least one vitamin, mineral, enzyme, amino acid, homeopathic supplement, toxin, homeopathic substance, traditional remedy, herbal supplement, or glandular supplement. In some embodiments, one or more receiving units 132 receive one or more signals 130 to select at least one vitamin, mineral, enzyme, amino acid, homeopathic supplement, toxin, homeopathic substance, traditional remedy, herbal supplement, or glandular supplement.

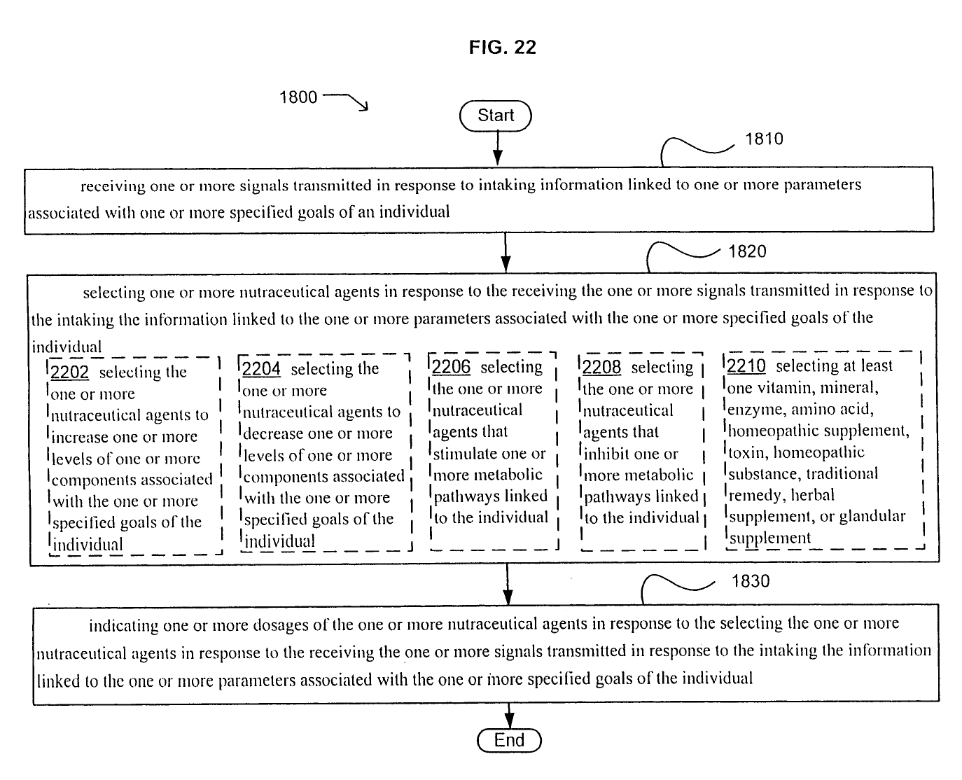

FIG. 22 illustrates alternative embodiments of the example operational flow 1800 of FIG. 18. FIG. 22 illustrates example embodiments where the selecting operation 1820 may include at least one additional operation. Additional operations may include an operation 2202, an operation 2204, an operation 2206, an operation 2208, and/or an operation 2210.

At operation 2202, the selecting operation 1820 may include selecting the one or more nutraceutical agents to increase one or more levels of one or more components associated with the one or more specified goals of the individual. In some embodiments, one or more receiving units 132 select one or more nutraceutical agents 118 to increase one or more levels of one or more components associated with the one or more specified goals 106 of the individual 108.

In some embodiments, one or more nutraceutical agents 118 may be selected that may directly complement one or more levels of one or more components associated with one or more specified coals 106 of the individual 108. For example, calcium in various forms (i.e., calcium citrate, calcium aspartate, calcium carbonate) may be selected by an individual 108 who wants to avoid or reduce the effects of osteoporosis. In another embodiment, chromium may be selected to lower blood sugar, increase insulin sensitivity, reduce body fat, control hunger, suppress appetite, increase lean body/muscle mass, or substantially any combination thereof. In some embodiments, one or more nutraceutical agents 118 associated with one or more specified goals 106 of an individual 108 may be selected that may be administered to an individual 108 to indirectly supplement a component associated with the one or more specified goals 106. For example, 5-hydroxy-tryptophan may be selected for administration to an individual 108 if the individual 108 suffers from depression and is found to exhibit low levels of serotonin. The 5-hydroxy-tryptophan will be converted to serotonin following administration to the individual 108. Numerous nutraceutical agents 118 may be selected to increase one or more levels of one or more components associated with one or more specified goals 106 of an individual 108.

At operation 2204, the selecting operation 1820 may include selecting the one or more nutraceutical agents to decrease one or more levels of one or more components associated with the one or more specified goals of the individual. In some embodiments, one or more receiving units 132 select one or more nutraceutical agents 118 to decrease one or more levels of one or more components associated with the one or more specified signals 106 of the individual 108.

In some embodiments, one or more components may be associated with one or more specified goals 106 of an individual 108. Low density lipoproteins are examples of components that are linked to vascular disease. Accordingly, in some embodiments, one or more nutraceutical agents 118 may be selected that will act to lower the low density lipoprotein concentration of an individual 108. Examples of such nutraceutical agents 118 include, but are not limited to, plant stanols, dietary fiber, stanol esters, isoflavones, diallyl sulfides, niacin, soy protein, and substantially any combination thereof. Glucose is an example of a component that is linked to hyperglycemia. Accordingly, in some embodiments, buckwheat is an example of a nutraceutical agent 118 that may be selected to lower blood glucose levels of an individual 108. Free radical oxygen species that include singlet oxygen, hydroxyl radicals, peroxides, and superoxide radicals are components that act to damage lipids and other cellular structures. Accordingly, one or more nutraceutical agents 118 may be selected to reduce levels of free radicals within an individual 108. Examples of such nutraceutical agents 118 include, but are not limited to, terpenes, carotenoids, limonoids, phenols, flavonoids, isoprenoids, and the like. Numerous other nutraceutical agents 118 may be selected to decrease one or more levels of one or more components associated with the one or more specified goals 106 of the individual 108.

At operation 2206, the selecting operation 1820 may include selecting the one or more nutraceutical agents that stimulate one or more metabolic pathways linked to the individual. In some embodiments, one or more receiving units 132 select one or more nutraceutical agents 118 that stimulate one or more metabolic pathways linked to the individual 108.

In some embodiments, stimulation of one or more metabolic pathways may be associated with one or more specified goals 106 of an individual 108. For example, in some embodiments, decline in L-carnitine synthesis is thought to be linked to senile reduction in bone synthesis (Colucci et al., Calcified Tissue International, 76:458-465 (2005)). Dihydroepiandrosterone (DHEA) is thought to affect levels of L-carnitine through promoting the expression of carnitine-synthesizing enzymes (Chiu et al., Calcified Tissue International, 64:527-533 (1999)). Accordingly, DHEA may be selected to stimulate the L-carnitine synthetic pathway to reduce senile reduction in bone synthesis. In other embodiments, buckwheat extract may be selected to lower blood glucose due to the ability of buckwheat extract to activate mitogen activated protein kinase via phospholipase D (Appleton and Lockwood, The Pharmaceutical Journal, 277:78-83 (2006)). In another embodiment, resveratrol has been shown to stimulate endothelial nitric oxide synthase activity (Klinge et al., J. Biol. Chem., 280(9):7460-7468 (2005); Wallerath et al., Circulation, 106(13):1652-1658 (2002)). Endothelial nitric oxide synthase is an enzyme that catalyzes the formation of nitric oxide by vascular endothelial cells. Nitric oxide is needed to maintain arterial relaxation (vasodilation), and impaired nitric oxide-dependent vasodilation is associated with increased risk of cardiovascular disease (Duffy and Vita, Curr. Opin. Lipidol., 14(1):21-27 (2003)). Accordingly, in some embodiments, resveratrol may be selected to reduce cardiovascular disease. Numerous nutraceutical agents 118 may be selected to one or more metabolic pathways linked to the individual 108.

At operation 2208, the selecting operation 1820 may include selecting the one or more nutraceutical agents that inhibit one or more metabolic pathways linked to the individual. In some embodiments, one or more receiving, units 132 select one or more nutraceutical agents 118 that inhibit one or more metabolic pathways linked to the individual 108.

In some embodiments, one or more metabolic pathways may be inhibited through inhibition of one or more enzymes that participate within the metabolic pathways Such metabolic pathways may be involved with a specified goal 106 of an individual 108. For example, angiotensin-converting enzyme has been linked to high blood pressure. Flavonoids have been shown to inhibit angiotensin-converting enzyme (Actis-Goretta et al., J. Agric. Food Chem., 54(1):229-234 (2006)). Accordingly, flavonoids may be selected to assist in the reduction of blood pressure (Li et al., Chin. J. Physiol., 48(2):101-106 (2005); Machha and Mustafa, J. Cardiovasc. Pharmacol., 46(1):36-40(2005)). Genistein, one of the predominant soy isoflavones, has been shown to compete with 17beta-estradiol for estrogen receptor binding because of its structural similarity, resulting in agonistic or antagonistic activity. This has been shown to cause inhibition of cell growth in breast and prostate cancers in vivo and in vitro. Accordingly, soy isoflavones containing genistein may be selected as a nutraceutical agent 118 for cancer chemoprevention (Sarkar et al., Mini Rev. Med. Chem., 6(4):401-407(2006)). The isoflavonoids, daidzin, daidzein, and puerarin have been shown to reduce alcohol consumption (Lin R C, Alcohol Clin. Exp. Res., 20(4):659-663 (1996)). A link between daidzin's capacity to reduce alcohol consumption and its ability to increase the liver mitochondrial monoamine oxidase: aldehyde dehydrogenase activity ratio has been established (Keung, Med. Res. Rev., 23(6):669-696 (2003)). This increase in ratio is thought to occur through inhibition of aldehyde dehydrogenase activity. Accordingly, isoflavonoid mixtures that include daidzin, daidzein, and/or puerarin may be selected to lower alcohol consumption. Numerous other nutraceutical agents 118 may be selected that inhibit one or more metabolic pathways linked to the individual 108.

At operation 2210, the selecting operation 1820 may include selecting at least one vitamin, mineral, enzyme, amino acid, homeopathic supplement, toxin, homeopathic substance, traditional remedy, herbal supplement, or glandular supplement. In some embodiments, one or more receiving units 132 select at least one vitamin, mineral, enzyme, amino acid, homeopathic supplement, toxin, homeopathic substance, traditional remedy, herbal supplement, or glandular supplement.

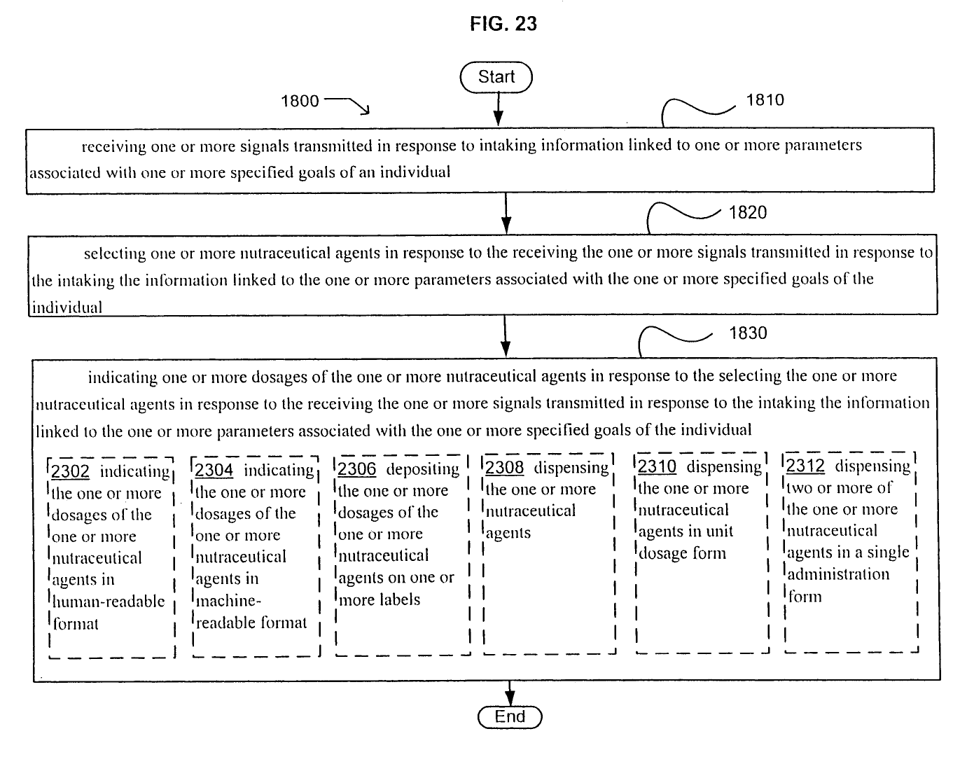

FIG. 23 illustrates alternative embodiments of the example operational flow 1800 of FIG. 8. FIG. 23 illustrates example embodiments where the indicating operation 1830 may include at least one additional operation. Additional operations may include an operation 2302, an operation 2304, an operation 2306, an operation 2308, an operation 2310, and/or an operation 2312.

At operation 2302, the indicating operation 1830 may include indicating the one or more dosages of the one or more nutraceutical agents in human-readable format. In some embodiments, one or more indicating units 120 indicate the one or more dosages 122 of the one or more nutraceutical agents 118 in human-readable format

In some embodiments, one or more indicating units 120 may indicate one or more dosages 122 of one or more nutraceutical agents 118 on a visual display, a touch display, an audible display, and the like. For example, in some embodiments, one or more indicating units 120 may display one or more dosages 122 on a light emitting diode display, a liquid crystal display, various monitors, and the like. Such visual displays may indicate one or more dosages 122 through display of colors, pictures, printed language, and the like. In some embodiments, one or more indicating units 120 may display one or more dosages 122 on a touch display (i.e., a touch pad display in Braille for use by blind or visually impaired persons). In some embodiments, one or more indicating units 120 may display one or more dosages 122 through use of an audible display that verbally speaks to an individual 108.

In some embodiments, one or more indicating units 120 may display one or more dosages 122 of one or more nutraceutical agents 118 in typographical symbols in numerous languages that are in human-readable format. In some embodiments, one or more indicating units 120 may display one or more dosages 122 of one or more nutraceutical agents 118 and the formulation type of the one or more nutraceutical agents 118 in typographical symbols in numerous languages that are in human-readable format. In some embodiments, one or more indicating units 120 may display one or more dosages 122 of one or more nutraceutical agents 118 in pictographic form that is in human-readable format. In some embodiments, one or more indicating units 120 mav display one or more dosages 122 of one or more nutraceutical agents 118 and the formulation type of the one or more nutraceutical agents 118 in pictographic form that is in human-readable format. In some embodiments, one or more indicating units 120 may display one or more dosages 122 of one or more nutraceutical agents 118 according to a color scheme that is in human-readable format. In some embodiments, one or more indicating units 120 may display one or more dosages 122 of one or more nutraceutical agents 118 and the formulation type of the one or more nutraceutical agents 118 according to a color scheme that is in human-readable format. In some embodiments, one or more indicating units 120 may display one or more dosages 122 of one or more nutraceutical agents 118, one or more routes of administration for the one or more nutraceutical agents 118, one or more times and/or periodicities for administration of the one or more nutraceutical agents 118, one or more formulation types for the one or more nutraceutical agents 118, whether to administer the one or more nutraceutical agents 118 with or without food, whether to administer the one or more nutraceutical agents 118 with or without other nutraceutical agents 118 and/or pharmaceutical agents, and substantially any combination thereof. Dosages 122 may be displayed according to numerous methods that are known and have been described (i.e., U.S. patent application Ser. No. 11/474,109, entitled CUSTOMIZED VISUAL MARKING FOR MEDICATION LABELING, filed 23 Jun. 2006, herein incorporated by reference to the extent such subject matter is not inconsistent herewith). In some embodiments, one or more indicating units 120 may indicate one or more dosages 122 in human-readable format and machine-readable format.

At operation 2304, the indicating operation 1830 may include indicating the one or more dosages of the one or more nutraceutical agents in machine-readable format. In some embodiments, one or more indicating units 120 indicate the one or more dosages 122 of the one or more nutraceutical agents 118 in machine-readable format.

In some embodiments, one or more indicating units 120 may indicate one or more dosages 122 of one or more nutraceutical agents 118 in machine-readable format. Numerous methods may be used to indicate one or more dosages 122. Examples of such methods include, but are not limited to, radio frequency identification, bar coding, typographical methods, symbol based methods (i.e., use of symbols that represent dosages 122 and nutraceutical agents 118), optical methods (i.e., pulsed light), and the like.

In some embodiments, one or more indicating units 120 may indicate one or more dosages 122 of one or more nutraceutical agents 118 and the formulation type of the one or more nutraceutical agents 18 in machine-readable form. In some embodiments, one or more indicating units 120 may indicate one or more dosages 122 of one or more nutraceutical agents 118 in a pictographic form that is in machine-readable format. In some embodiments, one or more indicating units 120 may indicate one or more dosages 122 of one or more nutraceutical agents 118 and the formulation type of the one or more nutraceutical agents 118 in a pictographic form that is in machine-readable format. In some embodiments, one or more indicating units 120 may indicate one or more dosages 122 of one or more nutraceutical agents 118 according to a color scheme that is in machine-readable format. In some embodiments, one or more indicating units 120 may indicate one or more dosages 122 of one or more nutraceutical agents 118 and the formulation type of the one or more nutraceutical agents 118 according to a color scheme that is in machine-readable format. In some embodiments, one or more indicating units 120 may indicate one or more dosages 122 of one or more nutraceutical agents 118, one or more routes of administration for the one or more nutraceutical agents 118, one or more times and/or periodicities for administration of the one or more nutraceutical agents 118, one or more formulation types for the one or more nutraceutical agents 118, whether to administer the one or more nutraceutical agents 118 with or without food, whether to administer the one or more nutraceutical agents 118 with or without other nutraccutical agents 118 and/or pharmaceutical agents, and substantially any combination thereof. Numerous labeling methods are known and have been described that may be adapted into a machine-readable format (i.e., U.S. patent application Ser. No. 11/474,109, entitled CUSTOMIZED VISUAL MARKING FOR MEDICATION LABELING, filed 23 Jun. 2006, herein incorporated by reference to the extent such subject matter is not inconsistent herewith). In some embodiments, one or more indicating units 120 may indicate one or more dosages 122 in human-readable and in a machine-readable format.

At operation 2306, the indicating operation 1830 may include depositing the one or more dosages of the one or more nutraceutical agents on one or more labels. In some embodiments, one or more indicating units 120 deposit the one or more dosages 122 of the one or more nutraceutical agents 118 on one or more labels.

In some embodiments, one or more indicating units 120 may print one or more Labels that include one or more dosages 122 of one or more nutraceutical agents 118 and the formulation type of the one or more nutraceutical agents 118. In some embodiments, one or more indicating units 120 may print one or more labels that include one or more dosages 122 of one or more nutraceutical agents 118 in pictographic form. In some embodiments, one or more indicating units 120 may print one or more labels that include one or more dosages 122 of one or more nutraceutical agents 118 and the formulation type of the one or more nutraceutical agents 118 in pictographic form. In some embodiments, one or more indicating units 120 may print one or more labels that include one or more dosages 122 of one or more nutraceutical agents 118 according to a color scheme. In some embodiments, one or more indicating units 120 may print one or more labels that include one or more dosages 122 of one or more nutraceutical agents 118 and the formulation type of the one or more nutraceutical agents 118 according to a color scheme. In some -embodiments, one or more indicating units 120 may indicate one or more dosages 122 of one or more nutraceutical agents 118, one or more routes of administration for the one or more nutraceutical agents 118, one or more times and/or periodicities for administration of the one or more nutraceutical agents 118, one or more formulation types for the one or more nutraceutical agents 118, whether to administer the one or more nutraceutical agents 118 with or without food, whether to administer the one or more nutraceutical agents 118 with or without other nutraceutical agents 118 and/or pharmaceutical agents, and substantially any combination thereof. Numerous labeling methods are known and have been described which may be adapted into machine-readable form (i.e., U.S. patent application Ser. No. 11/474,109, entitled CUSTOMIZED VISUAL MARKING FOR MEDICATION LABELING, filed 23 Jun. 2006, herein incorporated by reference to the extent such subject matter is not inconsistent herewith).

In some embodiments, one or more indicating units 120 may indicate one or more dosages 122 in human-readable and machine-readable form. Numerous types of depositing methods may be used by one or more indicating units 120. Examples of such methods include, but are not limited to, printing methods (i.e., stamping, ink-jet printing, laser printing, and the like). In some embodiments, ink containing magnetic particles may be used.

At operation 2308, the indicating operation 1830 may include dispensing the one or more nutraceutical agents. In some embodiments, one or more indicating units 120 dispense the one or more nutraceutical agents 118.

One or more indicating units 120 may dispense one or more nutraceutical agents 118 in numerous dosage forms. In some embodiments, one or more indicating units 120 may dispense one or more nutraceutical agents 118 in powder form. In some embodiments, one or more indicating units 120 may dispense one or more nutraceutical agents 118 in liquid form. In some embodiments, one or more indicating units 120 may dispense one or more nutraceutical agents 118 in tablet form. In some embodiments, one or more indicating units 120 may dispense one or more nutraceutical agents 118 in capsule form.

At operation 2310, the indicating operation 1830 may include dispensing the one or more nutraceutical agents in unit dosage form. In some embodiments, one or more indicating units 120 dispense the one or more nutraceutical agents 118 in unit dosage form.

In some embodiments, one or more indicating units 120 may dispense one or more nutraceutical agents 118 in a unit dosage form in which the entire dosage 122 is to be administered to an individual 108 as a single dosage 122. In some embodiments, one or more indicating units 120 may dispense one or more nutraceutical ac gents 118 in a unit dosage form that may be administered such that the individual 108 will maintain a level of the one or more nutraceuticals for a given time period. For example, in some embodiments, a unit dosage form may be an amount of one or more nutraceutical agents 118 that will allow the one or more nutraceutical agents 118 to be maintained within an individual 108 for four hours. Numerous criteria may be used to determine a unit dosage form. Examples of such criteria include, but are not limited to, physical characteristics of the individual 108, physiological characteristics of the individual 108, activity of the individual 108, and the like.

At operation 2312, the indicating operation 1830 may include dispensing two or more of the one or more nutraceutical agents in a single administration form. In some embodiments, one or more indicating units 120 dispense two or more of the one or more nutraceutical agents 118 in a single administration form.

In some embodiments, one or more indicating units 120 may dispense two or more nutraceutical agents 118 in a single administration form to provide for administration of the two or more nutraceutical agents 118 to an individual 108. Such methods have been described (i.e., U.S. patent application Ser. No. 11/453,571, filed 14 Jun. 2006; U.S. patent application Ser. No. 11/478,341, filed 28 Jun. 2006; U.S. patent application Ser. No. 11/478,296, filed 28 Jun. 2006; and U.S. patent application Ser. No. 11/486,998, filed 14 Jul. 2006, herein incorporated by reference to the extent such subject matter is not inconsistent herewith).

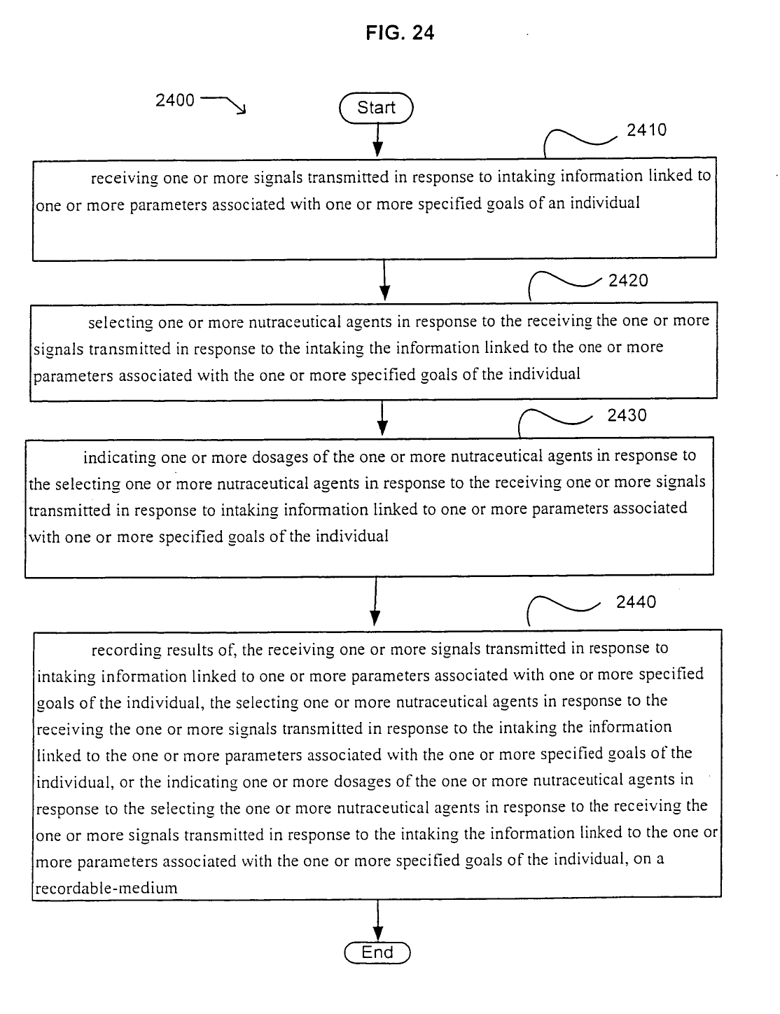

FIG. 24 illustrates an operational flow 2400 that includes a receiving operation 2410, a selecting operation 2420, and an indicating operation 2430 (which correspond to the receiving operation 1810, the selecting operation 1820, and the indicating operation 1830 illustrated in FIG. 18) with an additional recording operation 2440. In FIG. 24, discussion and explanation may be provided with respect to the above-described example of FIG. 1, and/or with respect to other examples and contexts. However, it should be understood that the operations may be executed in a number of other environments and contexts, and/or modified versions of FIG. 1. Also, although the various operations are presented in the sequence(s) illustrated, it should be understood that the various operations may be performed in other orders than those which are illustrated, or may be performed concurrently.

After a start operation, the operational flow 2400 includes the operations 2410, 2420, and 2430 (which correspond to operations 1810, 1820, and 1830 as described with regard to FIG.18) and an additional recording operation 2440 involving recording results of, the receiving one or more signals transmitted in response to intaking information linked to one or more parameters associated with one or more specified goals of an individual, the selecting one or more nutraceutical agents in response to the receiving the one or more signals transmitted in response to the intaking the information linked to the one or more parameters associated with the one or more specified goals of the individual, or the indicating one or more dosages of the one or more nutraceutical agents in response to the selecting the one or more nutraceutical agents in response to the receiving the one or more signals transmitted in response to the intaking the information linked to the one or more parameters associated with the one or more specified goals of the individual, on a recordable-medium.

In some embodiments, one or more recording units 124 may record results of, the receiving one or more signals 130 transmitted in response to intaking information 12 linked to one or more parameters 114 associated with one or more specified goals 106 of the individual 108, the selecting one or more nutraceutical agents 118 in response to the receiving one or more signals 110 transmitted in response to intakin q information 112 linked to one or more parameters 114 associated with one or more specified goals 106 of the individual 108, or the indicating one or more dosages 122 of the one or more nutraceutical agents 118 in response to the selecting one or more nutraceutical agents 118 in response to the receiving one or more signals 130 transmitted in response to intakin g information 112 linked to one or more parameters 114 associated with one or more specified goals 106 of the individual 108, on a recordable-medium 126.

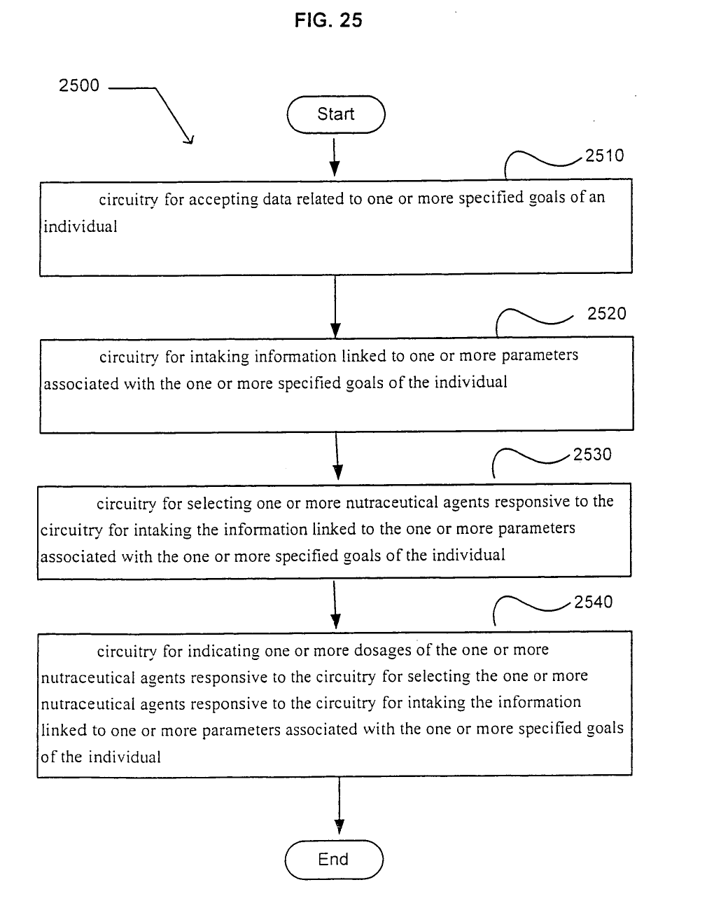

FIG. 25 illustrates a system 2500 representing examples of circuitry that is related to systems for nutraceutical agent 118 and dosing. In FIG. 25 discussion and explanation may be provided with respect to the above-described example of FIG. 1, and/or with respect to other examples and contexts. However, it should be understood that the circuitry may be assembled in a number of other environments and contexts, and/or modified versions of FIG. 1. Also, although various circuitry is presented in the sequence(s) illustrated, it should be understood that circuitry may be assembled in other configurations than those which are illustrated.

After a start operation, the system 2500 includes a circuitry block 2510 that includes circuitry for accepting data related to one or more specified goals of an individual. In some embodiments, the circuitry may be used for accepting data 104 related to one or more specified goals 106 of an individual 108.

The one or more specified goals 106 may be virtually any goal to be achieved, or attempted by, an individual 108 that may be affected by administration of one or more nutraceutical agents 118 to the individual 108. In some embodiments, administration of one or more nutraceutical agents 18 to an individual 108 may assist the individual 108 to achieve one or more specified goals 106. In some embodiments. administration of one or more nutraceutical agents 118 to an individual 108 may stimulate the individual 108 to achieve one or more specified goals 106. In some embodiments, administration of one or more nutraceutical agents 18 to an individual 108 may reduce an inhibition coupled to the individual 108 to promote achieving one or more specified goals 106 by the individual 108. Numerous examples of specified goals 106 of an individual 108 exist. In some embodiments, specified goals 106 of an individual 108 may be related to attentiveness, alertness, test performance, relaxation, pain, fever, attractiveness, anxiety, fall, injury, accident, bite, bleeding, inflammation, infection, drowsiness, insomnia, discomfort, stress, grooming, appearance, capability, performance, improvement, enhancement, curtailment, wellbeing, vitality, vigor, disability, phobia, malady, psychosis, environmental extremes, environmental exposure, dysfunction, disease symptom, chronic condition, mental acuity, emotional behavior, physical prowess, addiction, obsession, therapy, remedy, behavior, nutrition, diet, exercise, immunization, prevention, diagnosis, subscription, regimen, social performance, social interaction, endurance, sexual attribute, sexual performance, age-related attribute, age-related condition, prophylaxis, treatment, and substantially any combination thereof. In some embodiments, data 104 may include identification of one or more of the specified goals 106 of an individual 108. In some embodiments, data 104 may include characteristics of an individual 108. Examples of such data 104 may include, but are not limited to, physical characteristics, metabolic characteristics, financial characteristics, and the like. In some embodiments, data 104 may include, an individual's 108 height, weight, gender, kidney function, liver function, level of physical fitness, age, allergic response, metabolic level (i.e., resting metabolic rate and/or activity-related metabolic rate), disease state, body fat percentage, body mass index, personal health habits (i.e., smoking, alcohol consumption, diet, illegal drug use, and the like), family health history, insurance coverage, food supplement usage, nutraceutical agent 118 usage, non-prescription drug use, prescription drug use, pregnancy status, and the like. In some embodiments, the one or more characteristics may be specifically associated with an individual 108. As such, in some embodiments, the one or more characteristics may be unique to the individual l 08 as opposed to being common to a group. For example, in some embodiments, an individual 108 may be a member of a group of persons who are diabetic while exhibiting one or more characteristics, such as metabolic characteristics, that are unique to the individual 108. Accordingly, in some embodiments, data 104 may be input that provides for selection of nutraceutical agents 118 in accordance with one or more characteristics and specified goals 106 of an individual 108.

After a start operation, the system 2500 includes a circuitry block 2520 that includes circuitry for intakin a information linked to one or more parameters associated with the one or more specified goals of the individual. In some embodiments, the circuitry may be used for intaking information 112 linked to one or more parameters 114 associated with the one or more specified goals 106 of the individual 108.

Numerous parameters 114 may be associated with one or more specified goals 106 of an individual 108. Examples of such parameters 114 include, but are not limited to, blood sugar levels, blood insulin levels, blood iron levels, hormone levels (amine-derived hormones: such as catecholamines (adrenaline, dopamine, noradrenaline); tryptophan derivatives (melatonin, serotonin); tyrosine derivatives (thyroxine and triuodothyronine); peptide hormones such as antimullerian hormone, adiponectin, adrenocorticotropic hormone, angiotensinogen and angiotensin, antidiuretic hormone, atrial-natriuretic peptide, calcitonin, cholecystokinin, corticotropin-releasing hormone, erythropoietin, follicle-stimulating hormone, gastrin, ghrelin, glucagons, gonadotropin-releasing hormone, growth hormone-releasing hormone, human chorionic gonadotropin, growth hormone, inhibin, insulin, insulin-like growth factor, luteinizing hormone, melanocyte stimulating hormone, neuropeptide Y, oxytocin, parathyroid hormone, prolactin, relaxin, secretin, somatostatin, thrombopoietin, thyroid-stimulating hormone, thyrotropin-releasing hormone; steroid hormones: Glucocorticoids (cortisol); Mineralocorticoids (aldosterone); sex steroids: androgens (testosterone, dehydroepiandrosterone, dehydroepiandrosterone sulfate, androstenedione, dihydrotestosterone); estrogens (estradiol); progestagens (progesterone and progestins); sterol hormones: vitamin D derivatives (calcitriol); lipid hormones (prostaglandins, leukotrienes, prostacyclin, and thromboxane)), nutraceutical agent 118 levels, pharmaceutical agent levels, cytokine levels, and the like (i.e., Fitzgerald e t al., The Cytokine Facts Book, Academic Press, San Francisco, Second Edition, (2001)). Methods to gain information 112 with regard to components of biological systems are known (i.e., Clinical Laboratory Medicine, Lippincott Williams and Wilkins, Philadelphia, Second Edition, 2002). In some embodiments, one or more intaking units 110 may include instrumentation that provides for analysis of a sample obtained from an individual 108. For example, in some embodiments, an intaking unit 110 may be configured to intake a blood sample obtained from an individual 108 and analyze the blood sample to determine one or more parameters 114 associated with one or more specified goals 106 of an individual 108 (i.e., determine the level of free testosterone or the level of melatonin in a blood sample obtained from an individual 108). Numerous analytical technologies are known and may be included within one or more intaking units 110. Examples of such technologies include, but are not limited to, gas chromatography, mass spectrometry, atomic absorption, immunoassay based methods, microfluidic based methods, spectrophotometry (i.e., infrared, ultraviolet, fluorescence, and the like), surface plasmon resonance, fluorescence resonance energy transfer, and the like. In some embodiments, one or more intaking units 110 may intake information 112 linked to one or more parameters 114 associated with the one or more specified goals 106 of an individual 108 from a source that is independent of the one or more intaking units 110. In some embodiments, one or more intaking units 110 may intake information 112 linked to one or more parameters 114 associated with the one or more specified goals 106 of an individual 108 from a source that is coupled to the one or more intaking units 110.

After a start operation, the system 2500 includes a circuitry block 2530 that includes circuitry for selecting one or more nutraceutical agents responsive to the circuitry for intaking the information linked to the one or more parameters associated with the one or more specified goals of the individual. In some embodiments, the circuitry may be used for selecting one or more nutraceutical agents 118 responsive to the circuitry for intaking information 112 linked to one or more parameters 114 associated with the one or more specified goals 106 of the individual 108.

In some embodiments, one or more selecting units 116 may select one or more nutraceutical agents 118 in response to one or more intaking units 110. In some embodiments, one or more selecting units 116 may select one or more nutraceutical agents 118 in response to one or more accepting units 102. In some embodiments, one or more selecting units 116 may select one or more nutraceutical agents 118 in response to one or more intaking units 10 and one or more accepting units 102. Accordingly, in some embodiments, one or more selecting units 116 may select one or more nutraceutical agents 118 with regard to one or more characteristics of the individual 108 and one or more parameters 114 associated with one or more specified goals 106 of the individual 108. For example, in some embodiments, one or more selecting units 116 may select one or more nutraceutical agents 118 based on the age of the individual 108 and the level of testosterone in the individual's 108 blood. In other embodiments, one or more selecting units 116 may select one or more nutraceutical agents 118 based on insurance coverage held by an individual 108 and a specified goal 106 of the individual 108. Accordingly, numerous combinations of information 112 and data 104 may be used by one or more selecting units 116 to select one or more nutraceutical agents 118.

After a start operation, the system 2500 includes a circuitry block 2540 that includes circuitry for indicating one or more dosages of the one or more nutraceutical agents responsive to the circuitry for selecting the one or more nutraceutical agents responsive to the circuitry for intaking the information linked to the one or more parameters associated with the one or more specified goals of the individual. In some embodiments, the circuitry may be used for indicating one or more dosages 122 of the one or more nutraceutical agents 118 responsive to the circuitry for selecting one or more nutraceutical agents 118 responsive to the circuitry for intaking information 112 linked to one or more parameters 114 associated with the one or more specified goals 106 of the individual 108.

In some embodiments, the circuitry may be used for indicating one or more dosages 122 of one or more nutraceutical agents 118 in human-readable format. In some embodiments, the circuitry may be used for indicating one or more dosages 192 of one or more nutraceutical agents 118 in machine-readable format. In some embodiments, the circuitry may be used for depositing one or more dosages 122 of one or more nutraceutical agents 118 on one or more labels. In some embodiments, the circuitry may be used for dispensing one or more nutraceutical agents 118. In some embodiments, the circuitry may be used for dispensing one or more nutraceutical agents 118 in unit dosage form. In some embodiments, the circuitry may be used for dispensing two or more of one or more nutraceutical agents 118 in a single administration form.

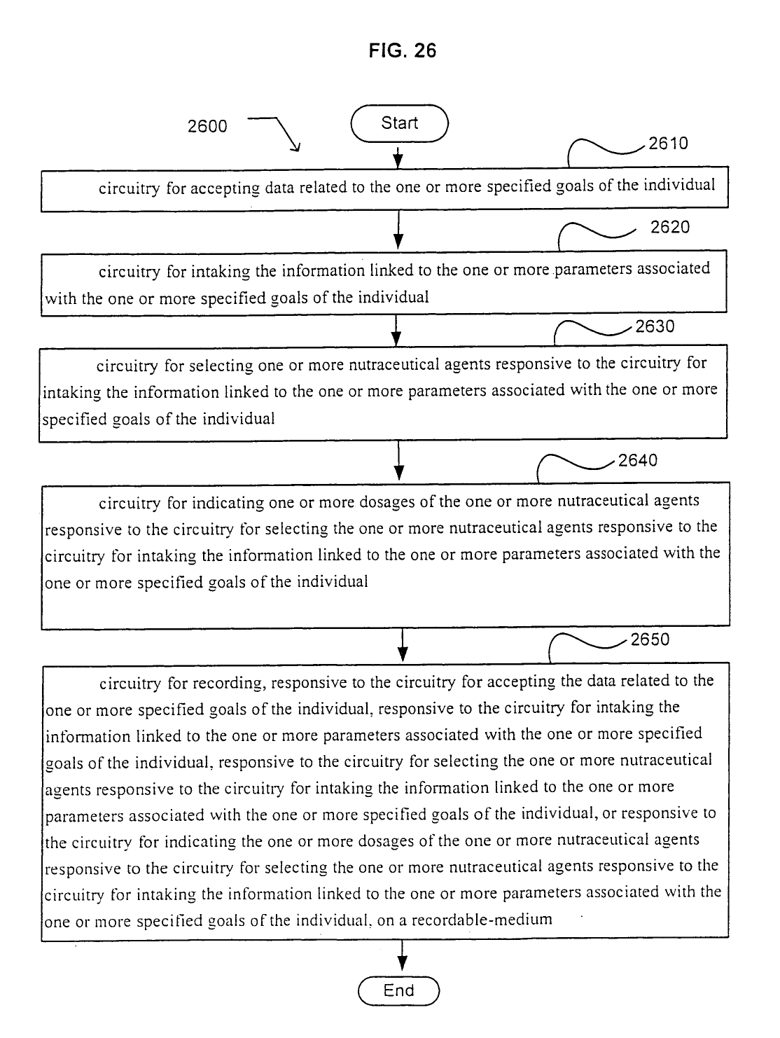

FIG. 26 illustrates a system 2600 representing examples of circuitry that is related to systems for nutraceutical agent 18 and dosing. In FIG. 26 discussion and explanation may be provided with respect to the above-described example of FIG. 1, and/or with respect to other examples and contexts. However, it should be understood that the circuitry may be assembled in a number of other environments and contexts, and/or modified versions of FIG. 1. Also, although various circuitry is presented in the sequence(s) illustrated, it should be understood that circuitry may be assembled in other configurations than those which are illustrated.

After a start operation, the operational flow 2600 includes circuitry blocks 2610, 2620, 2630, and 2640 (which correspond to circuitry blocks 2510, 2520, 2530, and 2540 as described with regard to FIG. 25) and an additional circuitry block 2650 involving circuitry for recording, responsive to the circuitry for accepting the data related to the one or more specified goals of the individual, responsive to the circuitry for intaking the information linked to the one or more parameters associated with the one or more specified goals of the individual, responsive to the circuitry for selecting the one or more nutraceutical agents responsive to the circuitry for intaking the information linked to the one or more parameters associated with the one or more specified goals of the individual, or responsive to the circuitry for indicating the one or more dosages of the one or more nutraceutical agents responsive to the circuitry for selecting the one or more nutraceutical agents responsive to the circuitry for intaking the information linked to the one or more parameters associated with the one or more specified goals of the individual, on a recordable-medium. In some embodiments, the circuitry may be used for recording, responsive to the circuitry for accepting data 104 related to one or more specified goals 106 of an individual 108, responsive to the circuitry for intaking information 112 linked to one or more parameters 114 associated with the one or more specified goals 106 of the individual 108, responsive to the circuitry for selecting one or more nutraceutical agents 118 responsive to the circuitry for intaking information 112 linked to one or more parameters 114 associated with the one or more specified goals 106 of the individual 108, or responsive to the circuitry for indicating one or more dosages 122 of the one or more nutraceutical agents 18 responsive to the circuitry for selecting one or more nutraceutical agents 118 responsive to the circuitry for intaking information 112 linked to one or more parameters 114 associated with the one or more specified goals 106 of the individual 108, on a recordable-medium 126.

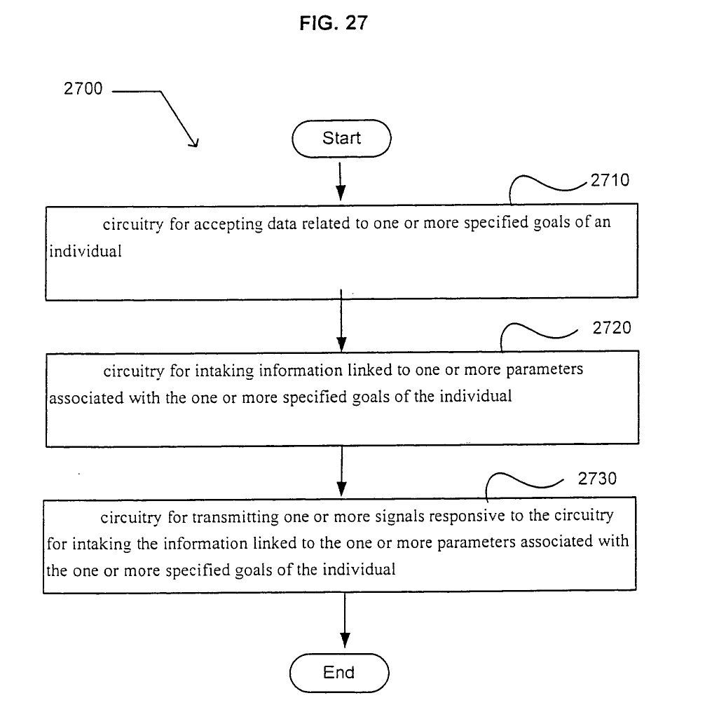

FIG. 27 illustrates a system 2700 representing examples of circuitry that is related to systems for nutraceutical agent 118 and dosing. In FIG. 27 discussion and explanation may be provided with respect to the above-described example of FIG. 1, and/or with respect to other examples and contexts. However, it should be understood that the circuitry may be assembled in a number of other environments and contexts, and/or modified versions of FIG. 1. Also, although various circuitry is presented in the sequence(s) illustrated, it should be understood that circuitry may be assembled in other configurations than those which are illustrated.

After a start operation, the system 2700 includes a circuitry block 2710 that includes circuitry for accepting data related to one or more specified goals of an individual. In some embodiments, the circuitry may be used for accepting data 104 related to one or more specified goals 106 of an individual 108.

The one or more specified goals 106 may be virtually any goal to be achieved, or attempted by, an individual 108 that may be affected by administration of one or more nutraceutical agents 118 to the individual 108. In some embodiments, administration of one or more nutraceutical agents 118 to an individual 108 may assist the individual 108 to achieve one or more specified goals 106. In some embodiments, administration of one or more nutraceutical agents 118 to an individual 108 may stimulate the individual 108 to achieve one or more specified goals 106. In some embodiments, administration of one or more nutraceutical agents 118 to an individual 108 may reduce an inhibition coupled to the individual 108 to promote achieving one or more specified goals 106 by the individual 108. Numerous examples of specified goals 106 of an individual 108 exist. In some embodiments, specified goals 106 of an individual 108 may be related to attentiveness, alertness, test performance, relaxation, pain, fever, attractiveness, anxiety, fall, injury, accident, bite, bleeding, inflammation, infection, drowsiness, insomnia, discomfort, stress, grooming, appearance, capability, performance, improvement, enhancement, curtailment, wellbeing, vitality, vigor, disability, phobia, malady, psychosis, environmental extremes, environmental exposure, dysfunction, disease symptom, chronic condition, mental acuity, emotional behavior, physical prowess, addiction, obsession, therapy, remedy, behavior, nutrition, diet, exercise, immunization, prevention, diagnosis, subscription, regimen, social performance, social interaction, endurance, sexual attribute, sexual performance, age-related attribute, a ge-related condition, prophylaxis, treatment, and substantially any combination thereof. In some embodiments, data 104 may include identification of one or more of the specified goals 106 of an individual 108. In some embodiments, data 104 may include characteristics of an individual 108. Examples of such data 104 may include, but are not limited to, physical characteristics, metabolic characteristics, financial characteristics, and the like. In some embodiments, data 104 may include, an individual's 108 height, weight, gender, kidney function, liver function, level of physical fitness, age, allergic response, metabolic level (i.e., resting metabolic rate and/or activity-related metabolic rate), disease state, body fat percentage, body mass index, personal health habits (i.e., smoking, alcohol consumption, diet, illegal drug use, and the like), family health history, insurance coverage, food supplement usage, nutraceutical agent 118 usage, non-prescription drug use, prescription drug use, pregnancy status, and the like. In some embodiments, the one or more characteristics may be specifically associated with an individual 108. As such, in some embodiments, the one or more characteristics may be unique to the individual 108 as opposed to being common to a group. For example, in some embodiments, an individual 108 may be a member of a group of persons who are diabetic while exhibiting one or more characteristics, such as metabolic characteristics, that are unique to the individual 108. Accordingly, in some embodiments, data 104 may be input that provides for selection of nutraceutical agents 118 in accordance with one or more characteristics and specified goals 106 of an individual 108.

After a start operation, the system 2700 includes a circuitry block 2720 that includes circuitry for intakin a information linked to one or more parameters associated with the one or more specified goals of the individual. In some embodiments, the circuitry may be used for intaking information 112 linked to one or more parameters 114 associated with the one or more specified goals 106 of the individual 108.

Numerous parameters 114 may be associated with one or more specified goals 106 of an individual 108. Examples of such parameters 114 include, but are not limited to, blood sugar levels, blood insulin levels, blood iron levels, hormone levels, nutraceutical agent 118 levels, pharmaceutical agent levels, cytokine levels, and the like (i.e., Fitzgerald et al., The Cytokine Facts Book, Academic Press, San Francisco, Second Edition, (2001)). Methods to gain information 112 with regard to components of biological systems are known (i.e., Clinical Laboratory Medicine, Lippincott Williams and Wilkins, Philadelphia, Second Edition, 2002). In some embodiments, one or more intaking units 110 may include circuitry for analysis of a sample obtained from an individual 108. For example, in some embodiments, an intaking unit 110 may include circuitry that is configured for analysis of a blood sample obtained from an individual 108 to determine one or more parameters 114 associated with one or more specified goals 106 of an individual 108 (i.e., determine the level of free testosterone or the level of melatonin in a blood sample obtained from an individual 108). Numerous analytical technologies are known and may be included within one or more intaking units 110. Examples of such technologies include, but are not limited to, gas chromatography, mass spectrometry, atomic absorption, immunoassay based methods, microfluidic based methods, spectrophotometry (i.e., infrared, ultraviolet, fluorescence, and the like), surface plasmon resonance, fluorescence resonance energy transfer, and the like. In some embodiments, the circuitry may be used to intake information 112 linked to one or more parameters 114 associated with the one or more specified goals 106 of an individual 108 from a source that is independent of the one or more intakin g units 110. In some embodiments, the circuitry may intake information 112 linked to one or more parameters 114 associated with the one or more specified goals 106 of an individual 108 from a source that is coupled to the one or more intaking units 110.

After a start operation, the system 2700 includes a circuitry block 2730 that includes circuitry for transmitting one or more signals responsive to the circuitry for intaking the information linked to the one or more parameters associated with the one or more specified goals of the individual. In some embodiments, the circuitry may be used for transmitting one or more signals 130 responsive to the circuitry for intakin a information 112 linked to one or more parameters 114 associated with the one or more specified goals 106 of the individual 108.

In some embodiments, the circuitry may be used for transmitting one or more levels of one or more metabolic indicators linked to the individual 108. In some embodiments, the circuitry may be used for transmitting one or more levels of one or more metabolic activities linked to the individual 108. In some embodiments, the circuitry may be used for transmitting one or more levels of one or more nutraceutical agents 118 linked to the individual 108. In some embodiments ! the circuitry may be used for transmitting one or more levels of one or more pharmaceutical agents linked to the individual 108. In some embodiments, the circuitry may be used for transmitting one or more parameters 114 associated with the one or more specified goals 106 of the individual 108. In some embodiments, the circuitry may be used for transmitting one or more instructions to select one or more nutraceutical agents 118 in response to the intaking information 112 linked to one or more parameters 114 associated with the one or more specified goals 106 of the individual 108. In some embodiments, the circuitry may be used for transmitting one or more signals 130 to select one or more nutraceutical agents 118 to increase one or more levels of one or more components associated with the one or more specified goals 106 of the individual 108. In some embodiments, the circuitry may be used for transmitting one or more signals 130 to select one or more nutraceutical agents 118 to decrease one or more levels of one or more components associated with the one or more specified goals 106 of the individual 108. In some embodiments, the circuitry may be used for transmitting one or more signals 130 to select one or more nutraceutical agents 118 that stimulate one or more metabolic pathways linked to the individual 108. In some embodiments, the circuitry may be used for transmitting one or more signals 130 to select one or more nutraceutical agents 118 that inhibit one or more metabolic pathways linked to the individual 108. In some embodiments, the circuitry may be used for transmitting one or more signals 130 to select at least one vitamin, mineral, enzyme, amino acid, homeopathic supplement, toxin, homeopathic substance, traditional remedy, herbal supplement, glandular supplement, or substantially any combination thereof.

In some embodiments, the circuitry may be used for transmitting one or more signals 130 that include data 104 related to one or more specified goals 106 of an individual 108. The circuitry used for transmitting one or more signals 130 may use numerous technologies. Examples of such technologies include, but are not limited to, wireless circuitry, telephone circuitry, internet circuitry, digital circuitry, analog circuitry, optical circuitry, and the like.

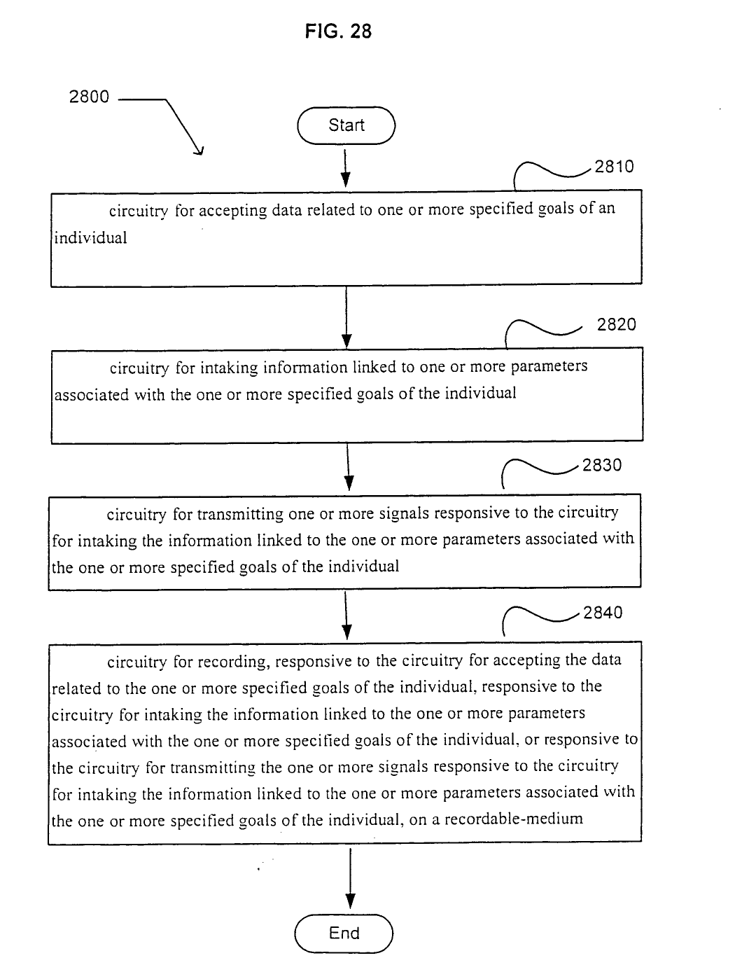

FIG. 28 illustrates a system 2800 representing examples of circuitry that is related to systems for nutraceutical agent 18 and dosing. In FIG. 28 discussion and explanation may be provided with respect to the above-described example of FIG. 1, and/or with respect to other examples and contexts. However, it should be understood that the circuitry may be assembled in a number of other environments and contexts, and/or modified versions of FIG. 1. Also, although various circuitry is presented in the sequence(s) illustrated, it should be understood that circuitry may be assembled in other configurations than those which are illustrated.

After a start operation, the operational flow 2800 includes circuitry blocks 2810, 2820, and 2830 (which correspond to circuitry blocks 2710, 2720, and 2730 as described with regard to FIG. 27) and an additional circuitry block 2840 involving circuitry for recording, responsive to the circuitry for accepting the data related to the one or more specified goals of the individual, responsive to the circuitry for intaking the information linked to the one or more parameters associated with the one or more specified goals of the individual, or responsive to the circuitry for transmitting the one or more signals responsive to the circuitry for intaking the information linked to the one or more parameters associated with the one or more specified goals of the individual, on a recordable-medium. In some embodiments, the circuitry may be used for recording, responsive to circuitry for accepting data 104 related to one or more specified goals 106 of an individual 108, responsive to circuitry for intaking information 112 linked to one or more parameters 114 associated with the one or more specified goals 106 of the individual 108, or responsive to circuitry for transmitting one or more signals 130 responsive to the circuitry for intaking information 112 linked to one or more parameters 114 associated with the one or more specified goals 106 of the individual 108, on a recordable-medium 126.

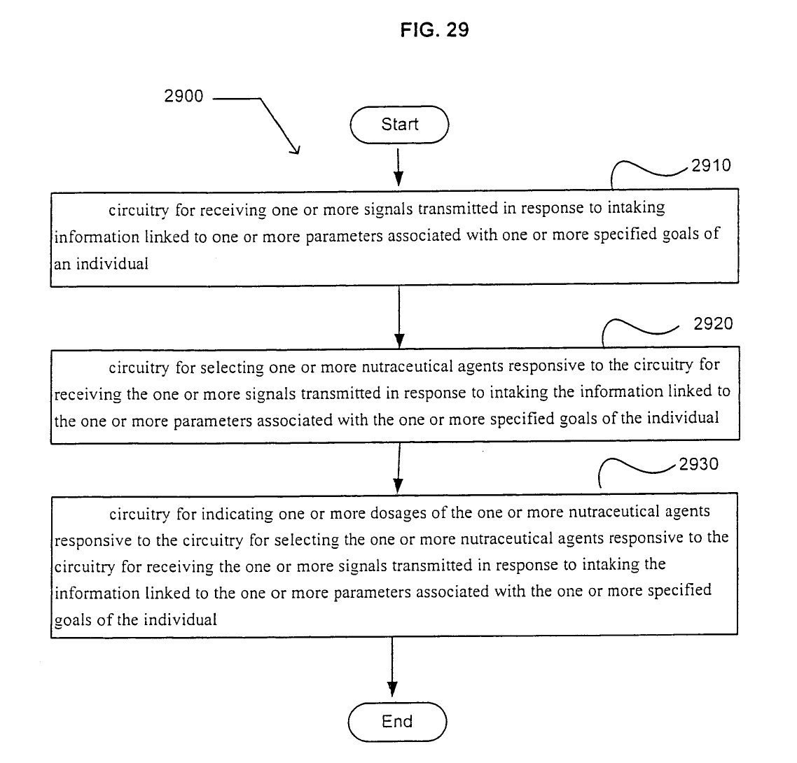

FIG. 29 illustrates a system 2900 representing examples of circuitry that is related to systems for nutraceutical agent 118 and dosing. In FIG. 29 discussion and explanation may be provided with respect to the above-described example of FIG. 1, and/or with respect to other examples and contexts. However, it should be understood that the circuitry may be assembled in a number of other environments and contexts, and/or modified versions of FIG. 1. Also, although various circuitry is presented in the sequence(s) illustrated, it should be understood that circuitry may be assembled in other configurations than those which are illustrated.

After a start operation, the system 2900 includes a circuitry block 2910 that includes circuitry for receiving one or more signals transmitted in response to intaking information linked to one or more parameters associated with one or more specified goals of an individual. In some embodiments, the circuitry may be used for receiving one or more signals 130 transmitted in response to intaking information 112 linked to one or more parameters 114 associated with one or more specified goals 106 of the individual 108.

In some embodiments, the circuitry may be used for receiving one or more signals 130 that include data 104 related to one or more specified goals 106 of an individual 108. In some embodiments, the circuitry may be used for receiving one or more signals 130 indicating one or more levels of one or more metabolic indicators linked to the individual 108. In some embodiments, the circuitry may be used for receiving one or more signals 130 indicating one or more metabolic activities linked to the individual 108. In some embodiments, the circuitry may be used for receiving one or more signals 130 indicating one or more levels of one or more nutraceutical agents 118 linked to the individual 108. In some embodiments, the circuitry may be used for receiving one or more signals 130 indicating one or more levels of one or more pharmaceutical agents linked to the individual 108. In some embodiments, the circuitry may be used for receiving one or more parameters 114 associated with the one or more specified goals 106 of the individual 108. In some embodiments, the circuitry may be used for receiving one or more instructions to select one or more nutraceutical agents 118 in response to the intakin a information 112 linked to one or more parameters 114 associated with the one or more specified goals 106 of the individual 108. In some embodiments, the circuitry may be used for receiving one or more signals 130 to select one or more nutraceutical agents 118 to increase one or more levels of one or more components associated with the one or more specified goals 106 of the individual 108. In some embodiments, the circuitry may be used for receiving one or more signals 130 to select one or more nutraceutical agents 118 to decrease one or more levels of one or more components associated with the one or more specified goals 106 of the individual 108. In some embodiments, the circuitry may be used for receiving one or more signals 130 to select one or more nutraceutical agents 118 that stimulate one or more metabolic pathways linked to the individual 108. In some embodiments, the circuitry may be used for receiving one or more signals 130 to select one or more nutraceutical agents 118 that inhibit one or more metabolic pathways linked to the individual 108. In some embodiments, the circuitry may be used for receiving one or more signals 130 to select at least one vitamin, mineral, enzyme, amino acid, homeopathic supplement, toxin, homeopathic substance, traditional remedy, herbal supplement, glandular supplement, or substantially any combination thereof.

In some embodiments, the circuitry may be used for receiving one or more signals 130 that include data 104 related to one or more specified goals 106 of an individual 108. The circuitry used for receiving may receive one or more signals 130 through use of numerous technologies. Examples of such technologies include, but are not limited to, wireless circuitry, telephone circuitry, internet circuitry, digital circuitry, analog circuitry, optical circuitry, and the like.

After a start operation, the system 2900 includes a circuitry block 2920 that includes circuitry for selecting one or more nutraceutical agents responsive to the circuitry for receiving the one or more signals transmitted in response to intaking the information linked to the one or more parameters associated with the one or more specified goals of the individual. In some embodiments, the circuitry may be used for selecting one or more nutraceutical agents 118 responsive to the circuitry for receiving one or more signals 130 transmitted in response to intaking information 112 linked to one or more parameters 114 associated with one or more specified goals 106 of the individual 108.

In some embodiments, the circuitry may be used for selecting one or more nutraceutical agents 118 in response to circuitry for receiving one or more signals 130 transmitted in response to intaking information 112 linked to one or more parameters 114 associated with one or more specified goals 106 of the individual 108. In some embodiments, circuitry may be used for selecting one or more nutraceutical agents 118 in response to circuitry for accepting. In some embodiments, the circuitry may be used for selecting one or more nutraceutical agents 118 in response to circuitry for intaking units 110 and circuitry for accepting. Accordingly, in some embodiments, circuitry may be used for selecting one or more nutraceutical agents 118 with regard to one or more characteristics of the individual 108 and one or more parameters 114 associated with one or more specified goals 106 of the individual 108. For example, in some embodiments, circuitry may be used for selecting one or more nutraceutical agents 118 based on the age of the individual 108 and the level of testosterone in the individual's 108 blood. In other embodiments, circuitry may be used for selecting one or more nutraceutical agents 118 based on insurance coverage held by an individual 108 and a specified goal 106 of the individual 108.

After a start operation, the system 2900 includes a circuitry block 2930 that includes circuitry for indicating one or more dosages of the one or more nutraceutical agents responsive to the circuitry for selecting the one or more nutraceutical agents responsive to the circuitry for receiving the one or more signals transmitted in response to intaking the information linked to the one or more parameters associated with the one or more specified goals of the individual. In some embodiments, the circuitry may be used for indicating one or more dosages 122 of the one or more nutraceutical agents 118 responsive to the circuitry for selecting one or more nutraceutical agents 118 responsive to the circuitry for receiving one or more signals 130 transmitted in response to intaking information 112 linked to one or more parameters 114 associated with one or more specified goals 106 of the individual 108.

In some embodiments, the circuitry may be used for indicating one or more dosages 122 of one or more nutraceutical agents 118 in human-readable format. In some embodiments, the circuitry may be used for indicating one or more dosages 122 of one or more nutraceutical agents 118 in machine-readable format. In some embodiments, the circuitry may be used for indicating one or more dosages 122 of one or more nutraceutical agents 118 on one or more labels. In some embodiments, the circuitry may be used for dispensing one or more nutraceutical agents 118. In some embodiments, the circuitry may be used for dispensing one or more nutraceutical agents 118 in unit dosage form. In some embodiments, the circuitry may be used for dispensing two or more of one or more nutraceutical agents 118 in a single administration form.

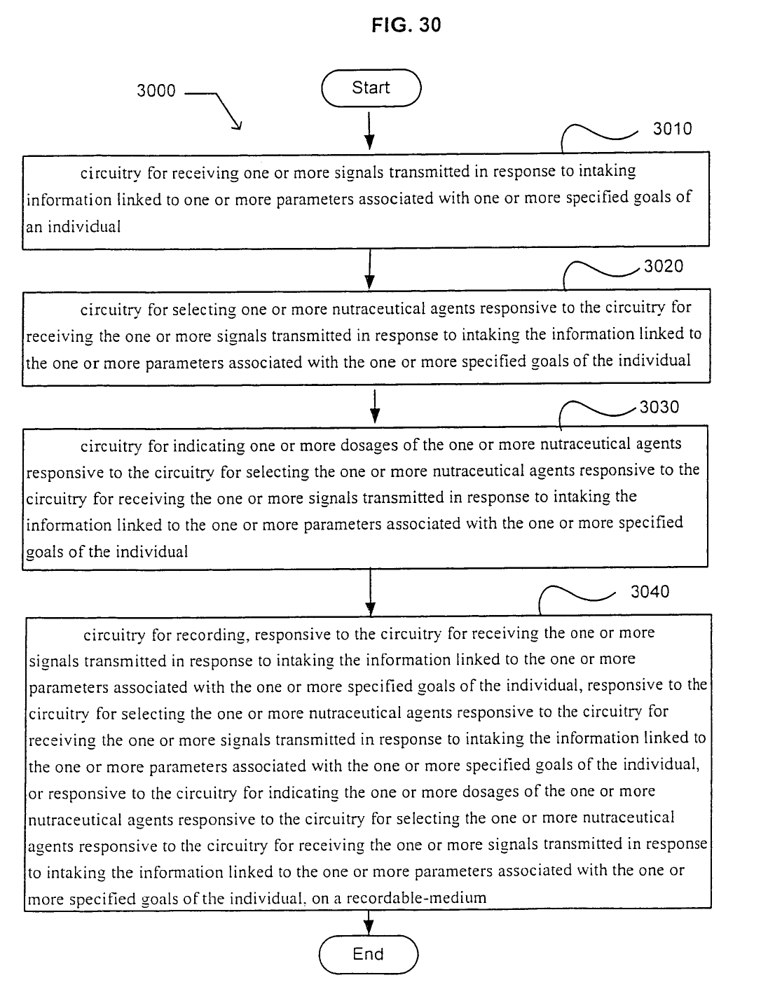

FIG. 30 illustrates a system 3000 representing examples of circuitry that is related to systems for nutraceutical agent 18 and dosing. In FIG. 30 discussion and explanation may be provided with respect to the above-described example of FIG. 1, and/or with respect to other examples and contexts. Flower, it should be understood that the circuitry may be assembled in a number of other environments and contexts, and/or modified versions of FIG. 1. Also, although various circuitry is presented in the sequence(s) illustrated, it should be understood that circuitry may be assembled in other configurations than those which are illustrated.

After a start operation, the operational flow 3000 includes circuitry blocks 3010, 3020, and 3030 (which correspond to circuitry blocks 2910, 2920, and 2930 as described with regard to FIG. 29) and an additional circuitry block 3040 involving circuitry for recording, responsive to the circuitry for receiving the one or more signals transmitted in response to intaking the information linked to the one or more parameters associated with the one or more specified goals of the individual, responsive to the circuitry for selecting the one or more nutraceutical agents responsive to the circuitry for receiving the one or more signals transmitted in response to intaking the information linked to the one or more parameters associated with the one or more specified goals of the individual, or responsive to the circuitry for indicating the one or more dosages of the one or more nutraceutical agents responsive to the circuitry for selecting the one or more nutraceutical agents responsive to the circuitry for receiving the one or more signals transmitted in response to intaking the information linked to the one or more parameters associated with the one or more specified goals of the individual, on a recordable-medium. In some embodiments, the circuitry may be used for recording, responsive to circuitry for receiving one or more signals 130 transmitted in response to intakin a information 112 linked to one or more parameters 114 associated with one or more specified goals 106 of the individual 108, responsive to the circuitry for selecting one or more nutraceutical agents 118 responsive to the circuitry for receiving one or more signals 130 transmitted in response to intaking information 112 linked to one or more parameters 114 associated with one or more specified goals 106 of the individual 108, or responsive to the circuitry for indicating one or more dosages 122 of the one or more nutraceutical agents 118 responsive to the circuitry for selecting one or more nutraceutical agents 118 responsive to the circuitry for receiving one or more signals 130 transmitted in response to intaking information 112 linked to one or more parameters 114 associated with one or more specified goals 106 of the individual 108, on a recordable-medium 126.

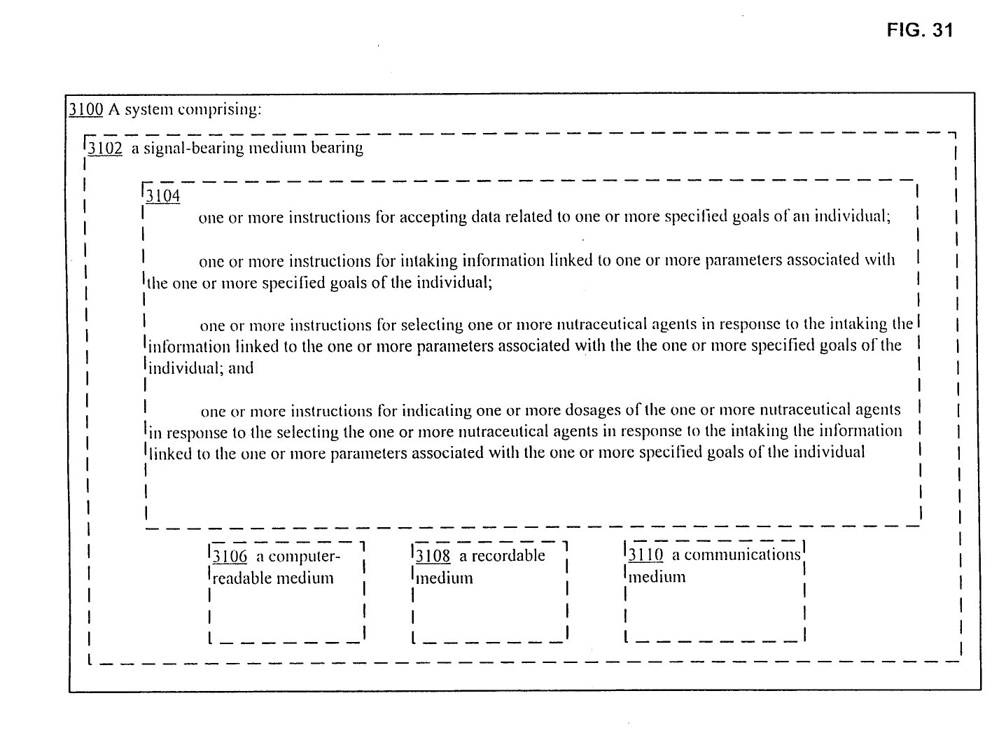

FIG. 31 illustrates a partial view of a system 3100 that includes a computer program 3104 for executing a computer process on a computing device. An embodiment of the system 3100 is provided using a signal-bearing medium bearing, one or more instructions for accepting data related to one or more specified goals of an individual, one or more instructions for intaking information linked to one or more parameters associated with the one or more specified goals of the individual, one or more instructions for selecting one or more nutraceutical agents in response to the intakin g the information linked to the one or more parameters associated with the one or more specified goals of the individual, and one or more instructions for indicating one or more dosages of the one or more nutraceutical agents in response to the selecting the one or more nutraceutical agents in response to the intaking the information linked to the one or more parameters associated with the one or more specified goals of the individual.

The one or more instructions may be, for example, computer executable and/or logic-implemented instructions. In some embodiments, the signal-bearing medium 3102 may include a computer-readable medium 3106. In some embodiments, the signal-bearing medium 3102 may include a recordable medium 3208. In some embodiments, the signal-bearing medium 3102 may include a communications medium 3110.

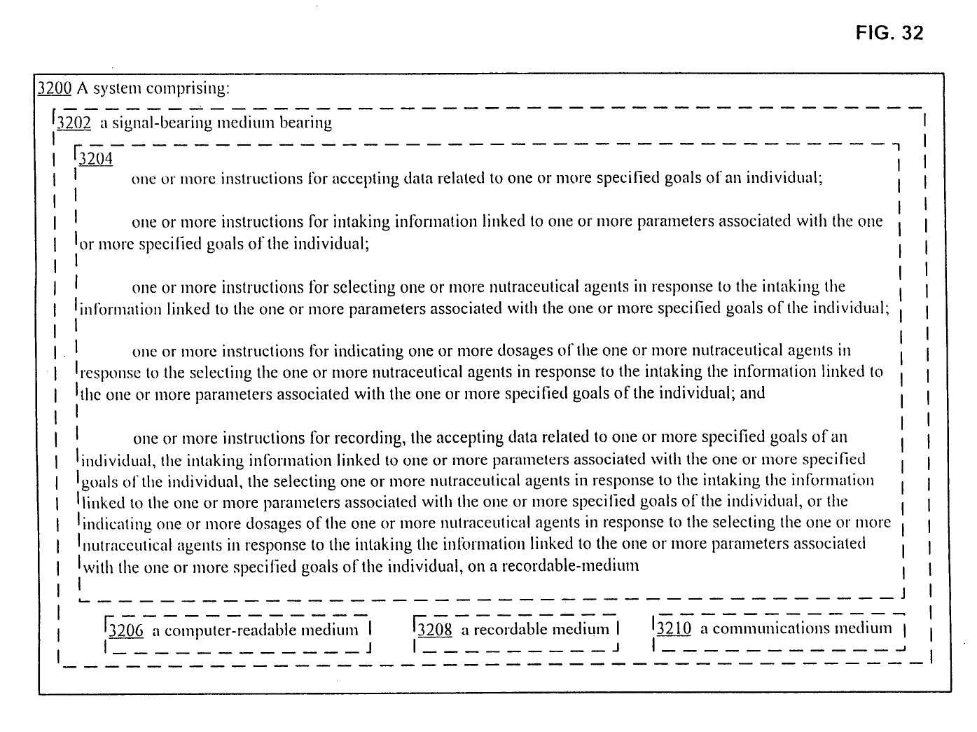

FIG. 32 illustrates a partial view of a system 3100 that includes a computer program 3204 for executing a computer process on a computing device. An embodiment of the system 3200 is provided using a signal-bearing medium bearing, one or more instructions for accepting data related to one or more specified goals of an individual, one or more instructions for intaking information linked to one or more parameters associated with the one or more specified goals of the individual, one or more instructions for selecting one or more nutraceutical agents in response to the intaking the information linked to the one or more parameters associated with the one or more specified goals of the individual, one or more instructions for indicating one or more dosages of the one or more nutraceutical agents in response to the selecting the one or more nutraceutical aoents in response to the intaking the information linked to the one or more parameters associated with the one or more specified goals of the individual, and one or more instructions for recording, the accepting data related to one or more specified goals of an individual, the intaking information linked to one or more parameters associated with the one or more specified goals of the individual, the selecting one or more nutraceutical agents in response to the intaking the information linked to the one or more parameters associated with the one or more specified goals of the individual, or the indicating one or more dosages of the one or more nutraceutical agents in response to the selecting the one or more nutraccutical agents in response to the intaking the information linked to the one or more parameters associated with the one or more specified goals of the individual, on a recordable-medium.

The one or more instructions may be, for example, computer executable and/or logic-implemented instructions. In some embodiments, the signal-bearing medium 3202 may include a computer-readable medium 3206. In some embodiments, the signal-bearing medium 3202 may include a recordable medium 3208. In some embodiments, the signal-bearing medium 3202 may include a communications medium 3210.

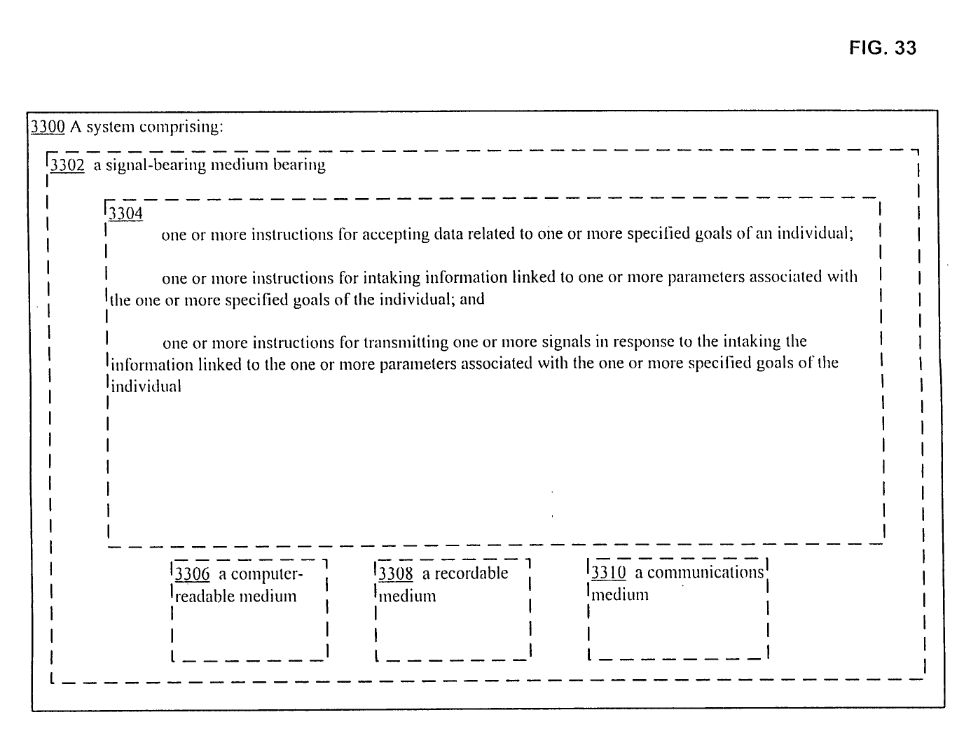

FIG. 33 illustrates a partial view of a system 3300 that includes a computer program 3304 for executing a computer process on a computing device. An embodiment of the system 3300 is provided using a signal-bearing medium bearing, one or more instructions for accepting data related to one or more specified goals of an individual, one or more instructions for intaking information linked to one or more parameters associated with the one or more specified goals of the individual, and one or more instructions for transmitting one or more signals in response to the intakin a the information linked to the one or more parameters associated with the one or more specified goals of the individual.

The one or more instructions may be, for example, computer executable and/or logic-implemented instructions. In some embodiments, the signal-bearing medium 3302 may include a computer-readable medium 3306. In some embodiments, the signal-bearing medium 3302 may include a recordable medium 3308. In some embodiments, the signal-bearing medium 3302 may include a communications medium 3310.

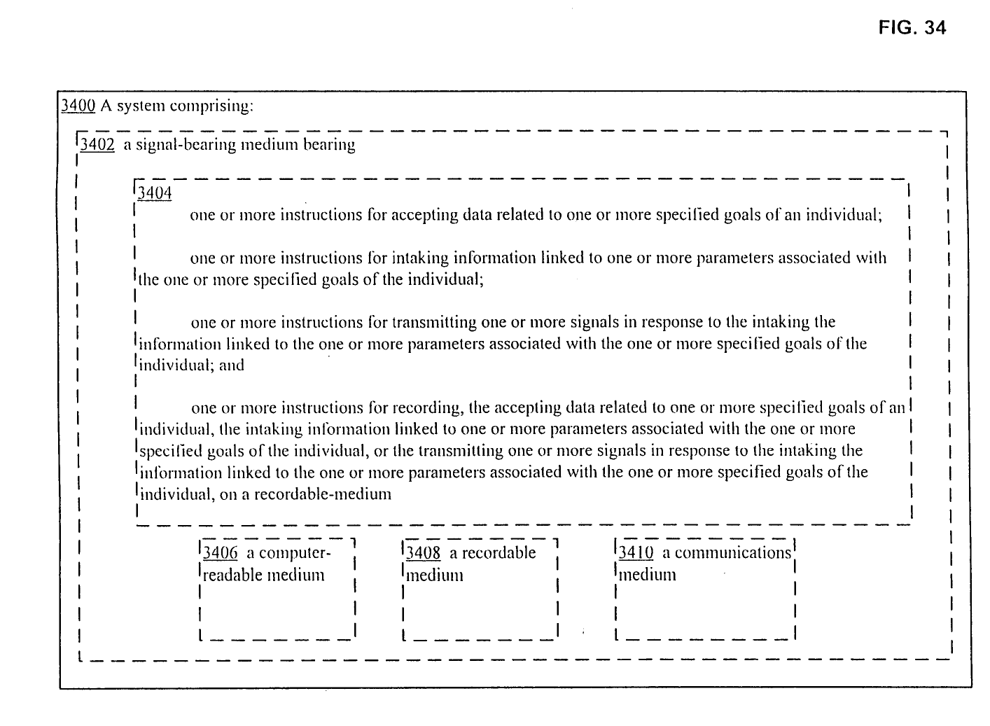

FIG. 34 illustrates a partial view of a system 3400 that includes a computer program 3404 for executing a computer process on a computing device. An embodiment of the system 3400 is provided using a signal-bearing medium bearing, one or more instructions for accepting data related to one or more specified goals of an individual, one or more instructions for intaking information linked to one or more parameters associated with the one or more specified goals of the individual, one or more instructions for transmitting one or more signals in response to the intaking, the information linked to the one or more parameters associated with the one or more specified goals of the individual, and one or more instructions for recording, the accepting data related to one or more specified goals of an individual, the intaking information linked to one or more parameters associated with the one or more specified goals of the individual, or the transmitting one or more signals in response to the intaking the information linked to the one or more parameters associated with the one or more specified goals of the individual, on a recordable-medium.

The one or more instructions may be, for example, computer executable and/or logic-implemented instructions. In some embodiments, the signal-bearing medium 3402 may include a computer-readable medium 3406. In some embodiments, the signal-bearing medium 3402 may include a recordable medium 3408. In some embodiments, the signal-bearing medium 3402 may include a communications medium 3410.

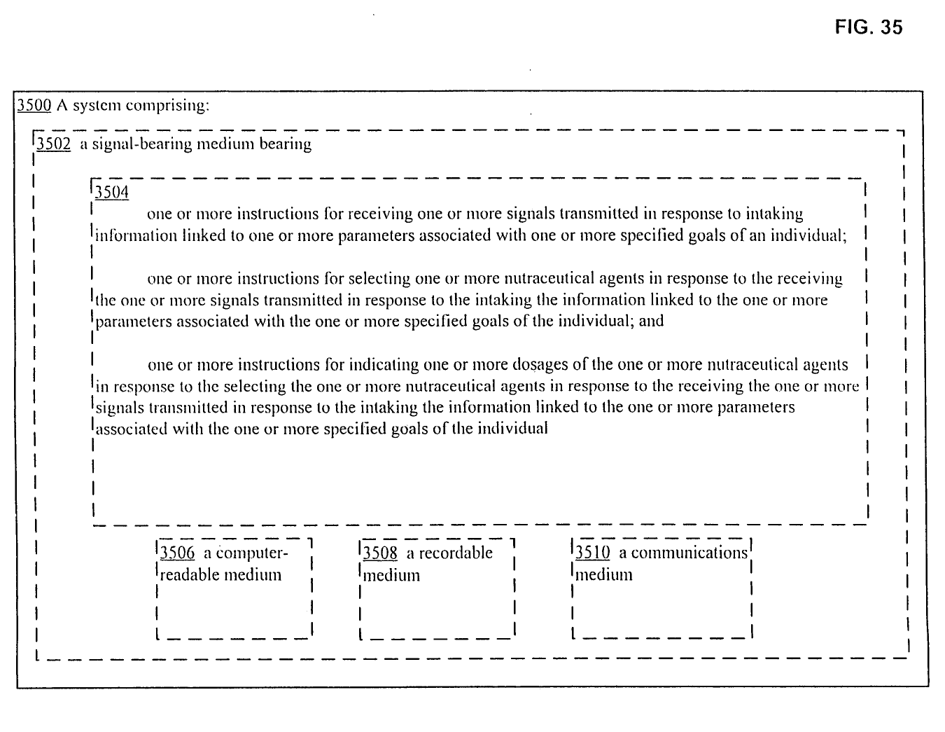

FIG. 35 illustrates a partial view of a system 3500 that includes a computer program 3504 for executing a computer process on a computing device. An embodiment of the system 3500 is provided using a signal-bearing medium bearing, one or more instructions for receiving one or more signals transmitted in response to intaking information linked to one or more parameters associated with one or more specified goals of an individual, one or more instructions for selecting one or more nutraceutical agents in response to the receiving the one or more signals transmitted in response to the intaking the information linked to the one or more parameters associated with the one or more specified goals of the individual, and one or more instructions for indicating one or more dosages of the one or more nutraceutical agents in response to the selecting the one or more nutraceutical agents in response to the receiving the one or more signals transmitted in response to the intaking the information linked to the one or more parameters associated with the one or more specified goals of the individual.

The one or more instructions may be, for example, computer executable and/or logic-implemented instructions. In some embodiments, the signal-bearing medium 3502 may include a computer-readable medium 3506. In some embodiments, the signal-bearing medium 3502 may include a recordable medium 3508. In some embodiments, the signal-bearing medium 3502 may include a communications medium 3510.

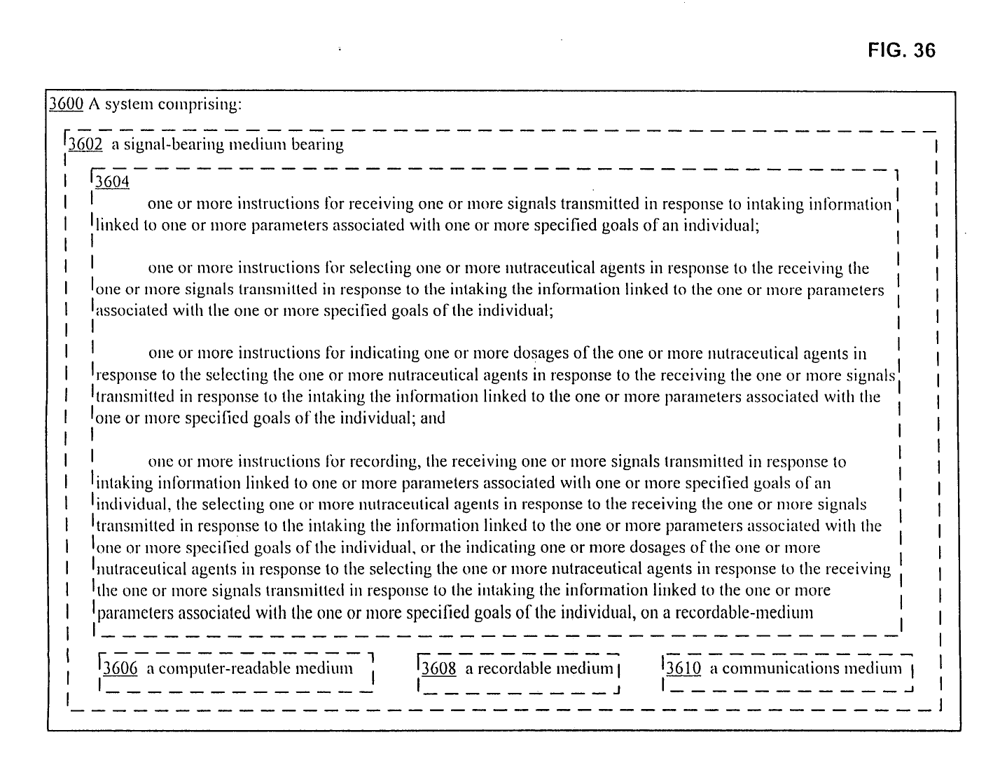

FIG. 36 illustrates a partial view of a system 3600 that includes a computer program 3604 for executing a computer process on a computing device. An embodiment of the system 3600 is provided using a signal-bearing medium bearing, one or more instructions for receiving one or more signals transmitted in response to intaking information linked to one or more parameters associated with one or more specified goals of an individual, one or more instructions for selecting one or more nutraceutical agents in response to the receiving the one or more signals transmitted in response to the intaking the information linked to the one or more parameters associated with the one or more specified goals of the individual, one or more instructions for indicating one or more dosages of the one or more nutraceutical agents in response to the selecting the one or more nutraceutical agents in response to the receiving the one or more signals transmitted in response to the intaking the information linked to the one or more parameters associated with the one or more specified goals of the individual, and one or more instructions for recording, the receiving one or more signals transmitted in response to intaking information linked to one or more parameters associated with one or more specified goals of an individual, the selecting one or more nutraceutical agents in response to the receiving the one or more signals transmitted in response to the intaking the information linked to the one or more parameters associated with the one or more specified goals of the individual, or the indicating one or more dosages of the one or more nutraceutical agents in response to the selecting the one or more nutraceutical agents in response to the receiving the one or more signals transmitted in response to the intaking the information linked to the one or more parameters associated with the one or more specified goals of the individual, on a recordable-medium.

The one or more instructions may be, for example, computer executable and/or logic-implemented instructions. In some embodiments, the signal-bearing medium 3602 may include a computer-readable medium 3606. In some embodiments, the signal-bearing medium 3602 may include a recordable medium 3608. In some embodiments, the signal-bearing medium 3602 may include a communications medium 3610.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e. g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims mav contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one ” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Further more, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal-bearing medium used to actually carry out the distribution. Examples of a signal-bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e. g., a fiber optic cable, a waveguide, a wired communications link, a wireless communication link, etc.).

In a general sense, those skilled in the art will recognize that the various embodiments described herein can be implemented, individually and/or collectively, by various types of electromechanical systems having a wide range of electrical components such as hardware, software, firmware, or virtually any combination thereof; and a wide range of components that may impart mechanical force or motion such as rigid bodies, spring or torsional bodies, hydraulics, and electro-magnetically actuated devices, or virtually any combination thereof. Consequently, as used herein “electro-mechanical system” includes, but is not limited to, electrical circuitry operably coupled with a transducer (e.g., an actuator, a motor, a piezoelectric crystal, etc.), electrical circuitry having at least one discrete electrical circuit, electrical circuitry having at least one integrated circuit, electrical circuitry having at least one application specific integrated circuit., electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e. g., forms of random access memory), electrical circuitry forming a communications device (e.g., a modem, communications switch, or optical-electrical equipment), and any non-electrical analog thereto, such as optical or other analogs. Those skilled in the art will also appreciate that examples of electromechanical systems include but are not limited to a variety of consumer electronics systems, as well as other systems such as motorized transport systems, factory automation systems, security systems, and communication/computing systems. Those skilled in the art will recognize that electromechanical as used herein is not necessarily limited to a system that has both electrical and mechanical actuation except as context mav dictate otherwise.

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

Those skilled in the art will recognize that it is common within the art to implement devices and/or processes and/or systems in the fashion(s) set forth herein, and thereafter use engineering and/or business practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation. Those having skill in the art will recognize that examples of such other devices and/or processes and/or systems might include—as appropriate to context and application—all or part of devices and/or processes and/or systems of (a) an air conveyance (e.g., an airplane, rocket, hovercraft, helicopter, etc.), (b) a ground conveyance (e.g., a car, truck, locomotive, tank, armored personnel carrier, etc.), (c) a building (e.g., a home, warehouse, office, etc.), (d) an appliance (e.g., a refrigerator, a washing machine, a dryer, etc.), (e) a communications system (e.g., a networked system, a telephone system, a voice-over IP system, etc.), (f) a business entity (e.g., an Internet Service Provider (ISP) entity such as Comcast Cable, Quest, Southwestern Bell, etc), or (g) a wired/wireless services entity (e.g., such as Sprint, Cingular, Nextel, etc.), etc.

Although user 136 is shown/described herein as a single illustrated figure, those skilled in the art will appreciate that a user 136 may be representative of a human user 136, a robotic user 136 (e.g., computational entity), and/or substantially any combination thereof (e.g., a user 136 may be assisted by one or more robotic agents). In addition, a user 136 as set forth herein, although shown as a single entity may in fact be composed of two or more entities. Those skilled in the art will appreciate that, in general, the same mav be said of “sender ” and/or other entity-oriented terms as such terms are used herein.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

All publications, patents and patent applications cited herein are incorporated herein by reference. The foregoing specification has been described in relation to certain embodiments thereof, and many details have been set forth for purposes of illustration, however, it will be apparent to those skilled in the art that the invention is susceptible to additional embodiments and that certain of the details described herein may be varied considerably without departing from the basic principles of the invention.

1.-78. (canceled)

79. A system comprising:
means for accepting data related to one or more specified goals of an individual;
means for intaking information linked to one or more parameters associated with the one or more specified goals of the individual;
means for selecting one or more nutraceutical agents responsive to the means for intaking the information linked to the one or more parameters associated with the one or more specified goals of the individual; and
means for indicating one or more dosages of the one or more nutraceutical agents responsive to the means for selecting the one or more nutraceutical agents responsive to the means for intaking the information linked to the one or more parameters associated with the one or more specified goals of the individual.

80. The system of claim 79, further comprising:
means for recording, responsive to the means for accepting the data related to the one or more specified goals of the individual, responsive to the means for intaking the information linked to the one or more parameters associated with the one or more specified goals of the individual, responsive to the means for selecting the one or more nutraceutical agents responsive to the means for intaking the information linked to the one or more parameters associated with the one or more specified goals of the individual, or responsive to the means for indicating the one or more dosages of the one or more nutraceutical agents responsive to the means for selecting the one or more nutraceutical agents responsive to the means for intaking the information linked to the one or more parameters associated with the one or more specified goals of the individual, on a recordable-medium.

81. A system comprising:
means for accepting data related to one or more specified goals of an individual;
means for intaking information linked to one or more parameters associated with the one or more specified goals of the individual; and
means for transmitting one or more signals responsive to the means for intaking the information linked to the one or more parameters associated with the one or more specified goals of the individual.

82. The system of claim 81, further comprising:
means for recording, responsive to the means for accepting the data related to the one or more specified goals of the individual, responsive to the means for intaking the information linked to the one or more parameters associated with the one or more specified goals of the individual, or responsive to the means for transmitting the one or more signals responsive to the means for intaking the information linked to the one or more parameters associated with the one or more specified goals of the individual, on a recordable-medium.

83. A system comprising:
means for receiving one or more signals transmitted in response to intaking information linked to one or more parameters associated with one or more specified goals of an individual;
means for selecting one or more nutraceutical agents responsive to the means for receiving the one or more signals transmitted in response to intaking the information linked to the one or more parameters associated with the one or more specified goals of the individual; and
means for indicating one or more dosages of the one or more nutraceutical agents responsive to the means for selecting the one or more nutraceutical agents responsive to the means for receiving the one or more signals transmitted in response to intaking the information linked to the one or more parameters associated with the one or more specified goals of the individual.

84. The system of claim 83, further comprising:
means for recording, responsive to the means for receiving the one or more signals transmitted in response to intaking the information linked to the one or more parameters associated with the one or more specified goals of the individual, responsive to the means for selecting the one or more nutraceutical agents responsive to the means for receiving the one or more signals transmitted in response to intaking the information linked to the one or more parameters associated with the one or more specified goals of the individual, or responsive to the means for indicating the one or more dosages of the one or more nutraceutical agents responsive to the means for selecting the one or more nutraceutical agents responsive to the means for receiving the one or more signals transmitted in response to intaking the information linked to the one or more parameters associated with the one or more specified goals of the individual, on a recordable-medium.

85. A system comprising:
a signal-bearing medium bearing:
one or more instructions for accepting data related to one or more specified goals of an individual;
one or more instructions for intaking information linked to one or more parameters associated with the one or more specified goals of the individual;
one or more instructions for selecting one or more nutraceutical agents in response to the intaking the information linked to the one or more parameters associated with the one or more specified goals of the individual; and
one or more instructions for indicating one or more dosages of the one or more nutraceutical agents in response to the selecting the one or more nutraceutical agents in response to the intaking the information linked to the one or more parameters associated with the one or more specified goals of the individual.

86. The system of claim 85, wherein the signal-bearing medium includes a computer-readable medium.

87. The system of claim 85, wherein the signal-bearing medium includes a recordable medium.

88. The system of claim 85, wherein the signal-bearing medium includes a communications medium.

89. The system of claim 85, further comprising:
one or more instructions for recording, the accepting data related to one or more specified goals of an individual, the intaking information linked to one or more parameters associated with the one or more specified goals of the individual, the selecting one or more nutraceutical agents in response to the intaking the information linked to the one or more parameters associated with the one or more specified goals of the individual, or the indicating one or more dosages of the one or more nutraceutical agents in response to the selecting the one or more nutraceutical agents in response to the intaking the information linked to the one or more parameters associated with the one or more specified goals of the individual, on a recordable-medium.

90. A system comprising:
a signal-bearing medium bearing:
one or more instructions for accepting data related to one or more specified goals of an individual;
one or more instructions for intaking information linked to one or more parameters associated with the one or more specified goals of the individual; and
one or more instructions for transmitting one or more signals in response to the intaking the information linked to the one or more parameters associated with the one or more specified goals of the individual.

91. The system of claim 90, wherein the signal-bearing medium includes a computer-readable medium.

92. The system of claim 90, wherein the signal-bearing medium includes a recordable medium.

93. The system of claim 90, wherein the signal-bearing medium includes a communications medium.

94. The system of claim 90, further comprising: one or more instructions for recording, the accepting data related to one or more specified goals of an individual, the intaking information linked to one or more parameters associated with the one or more specified goals of the individual, or the transmitting one or more signals in response to the intaking the information linked to the one or more parameters associated with the one or more specified goals of the individual, on a recordable-medium.

95. A system comprising:
a signal-bearing medium bearing:
one or more instructions for receiving one or more signals transmitted in response to intaking information linked to one or more parameters associated with one or more specified goals of an individual;
one or more instructions for selecting one or more nutraceutical agents in response to the receiving the one or more signals transmitted in response to the intaking the information linked to the one or more parameters associated with the one or more specified goals of the individual; and
one or more instructions for indicating one or more dosages of the one or more nutraceutical agents in response to the selecting the one or more nutraceutical agents in response to the receiving the one or more signals transmitted in response to the intaking the information linked to the one or more parameters associated with the one or more specified goals of the individual.

96. The system of claim 95, wherein the signal-bearing medium includes a computer-readable medium.

97. The system of claim 95, wherein the signal-bearing medium includes a recordable medium.

98. The system of claim 95, wherein the signal-bearing medium includes a communications medium.

99. The system of claim 95, further comprising:
one or more instructions for recording, the receiving one or more signals transmitted in response to intaking information linked to one or more parameters associated with one or more specified goals of an individual, the selecting one or more nutraceutical agents in response to the receiving the one or more signals transmitted in response to the intaking the information linked to the one or more parameters associated with the one or more specified goals of the individual, or the indicating one or more dosages of the one or more nutraceutical agents in response to the selecting the one or more nutraceutical agents in response to the receiving the one or more signals transmitted in response to the intaking the information linked to the one or more parameters associated with the one or more specified goals of the individual, on a recordable-medium.
